SEQUENCE LISTING (159 sequences)

SEQ ID NO: 1 - protein, 635 aa, Legionella quateirensis
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFNVEQIVRMVSHNGGSKNLKAVTDNHDDLKNMGFNAEQIVRMVSHGGGSKNLKAVTDNHDDLKNMGFNAEQIVSMVSNNGGSKNLKAVTDNHDDLKNMGFNAEQIVSMVSNGGGSLNLKAVKKYHDALKDRGFNTEQIVRMVSHDGGSLNLKAVKKYHDALRERKFNVEQIVSIVSHGGGSLNLKAVKKYHDVLKDREFNAEQIVRMVSHDGGSLNLKAVTDNHDDLKNMGFNAEQIVRMVSHKGGSKNLALVKEYFPVFSSFHFTADQIVALICQSKQCFRNLKKNHQQWKNKGLSAEQIVDLILQETPPKPNFNNTSSSTPSPSAPSFFQGPSTPIPTPVLDNSPAPIFSNPVCFFSSRSENNTEQYLQDSTLDLDSQLGDPTKNFNVNNFWSLFPFDDVGYHPHSNDVGYHLHSDEESPFFDF

SEQ ID NO: 2 - protein, 33 aa, Legionella quateirensis
FSSQQIIRMVSHAGGANNLKAVTANHDDLQNMG

SEQ ID NO: 3 - protein, 33 aa, Legionella quateirensis
FNVEQIVRMVSHNGGSKNLKAVTDNHDDLKNMG

SEQ ID NO: 4 - protein, 33 aa, Legionella quateirensis
FNAEQIVRMVSHGGGSKNLKAVTDNHDDLKNMG

SEQ ID NO: 5 - protein, 33 aa, Legionella quateirensis
FNAEQIVSMVSNNGGSKNLKAVTDNHDDLKNMG

SEQ ID NO: 6 - protein, 33 aa, Legionella quateirensis
FNAEQIVSMVSNGGGSLNLKAVKKYHDALKDRG

SEQ ID NO: 7 - protein, 33 aa, Legionella quateirensis
FNTEQIVRMVSHDGGSLNLKAVKKYHDALRERK

SEQ ID NO: 8 - protein, 33 aa, Legionella quateirensis
FNVEQIVSIVSHGGGSLNLKAVKKYHDVLKDRE

SEQ ID NO: 9 - protein, 33 aa, Legionella quateirensis
FNAEQIVRMVSHDGGSLNLKAVTDNHDDLKNMG

SEQ ID NO: 10 - protein, 19 aa, Legionella quateirensis
FNAEQIVRMVSHKGGSKNL

SEQ ID NO: 11 - protein, 196 aa, Flavobacterium okeanokoites
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 12 - protein, 176 aa, synthetic construct
ALVKEYFPVFSSFHFTADQIVALICQSKQCFRNLKKNHQQWKNKGLSAEQIVDLILQETPPKPNFNNTSSSTPSPSAPSFFQGPSTPIPTPVLDNSPAPIFSNPVCFFSSRSENNTEQYLQDSTLDLDSQLGDPTKNFNVNNFWSLFPFDDVGYHPHSNDVGYHLHSDEESPFFDF

SEQ ID NO: 13 - protein, 143 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDG

SEQ ID NO: 14-19 - skipped (6 entries)

SEQ ID NO: 20 - protein, 120 aa, synthetic construct
DATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDG

SEQ ID NO: 21 - protein, 115 aa, synthetic construct
NSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDG

SEQ ID NO: 22 - protein, 63 aa, synthetic construct
ALVKEYFPVFSSFHFTADQIVALICQSKQCFRNLKKNHQQWKNKGLSAEQIVDLILQETPPKP

SEQ ID NO: 23 - protein, 33 aa, Legionella maceachernii
FSAEQIVRIAAHDGGSRNIEAVQQAQHVLKELG

SEQ ID NO: 24 - protein, 33 aa, Legionella maceachernii
FSAEQIVSIVAHDGGSRNIEAVQQAQHILKELG

SEQ ID NO: 25 - protein, 33 aa, Legionellales bacterium RIFCSPHIGHO2_12_FULL_35_11
LDRQQILRIASHDGGSKNIAAVQKFLPKLMNFG

SEQ ID NO: 26 - protein, 33 aa, Legionella maceachernii
FSAEQIVRIAAHDGGSLNIDAVQQAQQALKELG

SEQ ID NO: 27 - protein, 33 aa, Legionella maceachernii
FSTEQIVCIAGHGGGSLNIKAVLLAQQALKDLG

SEQ ID NO: 28 - protein, 33 aa, Legionella maceachernii
YSSEQIVRVAAHGGGSLNIKAVLQAHQALKELD

SEQ ID NO: 29 - protein, 33 aa, Legionella maceachernii
FSAEQIVHIAAHGGGSLNIKAILQAHQTLKELN

SEQ ID NO: 30 - protein, 33 aa, Legionella maceachernii
FSAEQIVRIAAHIGGSRNIEAIQQAHHALKELG

SEQ ID NO: 31 - protein, 33 aa, Legionella maceachernii
FSAEQIVRIAAHIGGSHNLKAVLQAQQALKELD

SEQ ID NO: 32 - protein, 33 aa, Legionella maceachernii
FSAKHIVRIAAHIGGSLNIKAVQQAQQALKELG

SEQ ID NO: 33 - protein, 33 aa, Legionella quateirensis
FNAEQIVRMVSHKGGSKNLALVKEYFPVFSSFH

SEQ ID NO: 34 - protein, 33 aa, Legionella maceachernii
FSADQIVRIAAHKGGSHNIVAVQQAQQALKELD

SEQ ID NO: 35 - protein, 33 aa, Legionella maceachernii
FSAEQIVSIAAHVGGSHNIEAVQKAHQALKELD

SEQ ID NO: 36 - protein, 33 aa, Burkholderia sp.
FSSGETVGATVGAGGTETVAQGGTASNTTVSSG

SEQ ID NO: 37 - protein, 33 aa, Burkholderia sp.
FSGGMATSTTVGSGGTQDVLAGGAAVGGTVGTG

SEQ ID NO: 38 - protein, 33 aa, Burkholderia sp.
FSAADIVKIAGKIGGAQALQAFITHRAALIQAG

SEQ ID NO: 39 - protein, 33 aa, Burkholderia sp.
FNPTDIVKIAGNDGGAQALQAVLELEPALRERG

SEQ ID NO: 40 - protein, 33 aa, Burkholderia sp.
FNPTDIVRMAGNDGGAQALQAVFELEPAFRERS

SEQ ID NO: 41 - protein, 33 aa, Burkholderia sp.
FNPTDIVRMAGNDGGAQALQAVLELEPAFRERG

SEQ ID NO: 42 - protein, 33 aa, Burkholderia sp.
FSQVDIVKIASNDGGAQALYSVLDVEPTFRERG

SEQ ID NO: 43 - protein, 33 aa, Burkholderia sp.
FSRADIVKIAGNDGGAQALYSVLDVEPPLRERG

SEQ ID NO: 44 - protein, 33 aa, Burkholderia sp.
FSRGDIVKIAGNDGGAQALYSVLDVEPPLRERG

SEQ ID NO: 45 - protein, 33 aa, Burkholderia sp.
FNRADIVRIAGNGGGAQALYSVRDAGPTLGKRG

SEQ ID NO: 46 - protein, 33 aa, Burkholderia sp.
FRQADIVKIASNGGSAQALNAVIKLGPTLRQRG

SEQ ID NO: 47 - protein, 33 aa, Burkholderia sp.
FRQADIVKMASNGGSAQALNAVIKLGPTLRQRG

SEQ ID NO: 48 - protein, 33 aa, Burkholderia sp.
FSRADIVKIAGNGGGAQALQAVLELEPTFRERG

SEQ ID NO: 49 - protein, 33 aa, Burkholderia sp.
FSRADIVRIAGNGGGAQALYSVLDVGPTLGKRG

SEQ ID NO: 50 - protein, 33 aa, Burkholderia sp.
FSRGDIVRIAGNGGGAQALQAVLELEPTLGERG

SEQ ID NO: 51 - protein, 33 aa, Burkholderia sp.
FSRADIVKIAGNGGGAQALQAVITHRAALTQAG

SEQ ID NO: 52 - protein, 33 aa, Burkholderia sp.
FSRGDTVKIAGNIGGAQALQAVLELEPTLRERG

SEQ ID NO: 53 - protein, 33 aa, Burkholderia sp.
FNPTDIVKIAGNIGGAQALQAVLELEPAFRERG

SEQ ID NO: 54 - protein, 33 aa, Burkholderia sp.
FSAADIVKIAGNIGGAQALQAIFTHRAALIQAG

SEQ ID NO: 55 - protein, 33 aa, Burkholderia sp.
FSAADIVKIAGNIGGAQALQAVITHRATLTQAG

SEQ ID NO: 56 - protein, 33 aa, Burkholderia sp.
FSATDIVKIASNIGGAQALQAVISRRAALIQAG

SEQ ID NO: 57 - protein, 33 aa, Burkholderia sp.
FSQPDIVKIAGNIGGAQALQAVLELEPAFRERG

SEQ ID NO: 58 - protein, 33 aa, Burkholderia sp.
FSRADIVKIAGNIGGAQALQAVLELESTFRERS

SEQ ID NO: 59 - protein, 33 aa, Burkholderia sp.
FSRADIVKIAGNIGGAQALQAVLELESTLRERS

SEQ ID NO: 60 - protein, 33 aa, Burkholderia sp.
FSRGDIVKMAGNIGGAQALQAGLELEPAFRERG

SEQ ID NO: 61 - protein, 33 aa, Burkholderia sp.
FSRGDIVKMAGNIGGAQALQAVLELEPAFHERS

SEQ ID NO: 62 - protein, 33 aa, Burkholderia sp.
FTLTDIVKMAGNIGGAQALKAVLEHGPTLRQRD

SEQ ID NO: 63 - protein, 33 aa, Burkholderia sp.
FTLTDIVKMAGNIGGAQALKVVLEHGPTLRQRD

SEQ ID NO: 64 - protein, 33 aa, Burkholderia sp.
FNPTDIVKIAGNNGGAQALQAVLELEPALRERG

SEQ ID NO: 65 - protein, 33 aa, Burkholderia sp.
FNPTDIVKIAGNNGGAQALQAVLELEPALRERS

SEQ ID NO: 66 - protein, 33 aa, Burkholderia sp.
FNPTDMVKIAGNNGGAQALQAVLELEPALRERG

SEQ ID NO: 67 - protein, 33 aa, Burkholderia sp.
FSAADIVKIASNNGGAQALQALIDHWSTLSGKT

SEQ ID NO: 68 - protein, 33 aa, Burkholderia sp.
FSAADIVKIASNNGGAQALQAVISRRAALIQAG

SEQ ID NO: 69 - protein, 33 aa, Burkholderia sp.
FSAADIVKIASNNGGAQALQAVITHRAALAQAG

SEQ ID NO: 70 - protein, 33 aa, Burkholderia sp.
FSAADIVKIASNNGGARALQALIDHWSTLSGKT

SEQ ID NO: 71 - protein, 33 aa, Burkholderia sp.
FTLTDIVEMAGNNGGAQALKAVLEHGSTLDERG

SEQ ID NO: 72 - protein, 33 aa, Burkholderia sp.
FTLTDIVKMAGNNGGAQALKAVLEHGPTLDERG

SEQ ID NO: 73 - protein, 33 aa, Burkholderia sp.
FTLTDIVKMAGNNGGAQALKVVLEHGPTLRQRG

SEQ ID NO: 74 - protein, 33 aa, Burkholderia sp.
FTLTDIVKMASNNGGAQALKAVLEHGPTLDERG

SEQ ID NO: 75 - protein, 33 aa, Burkholderia sp.
FSAADIVKIAGNSGGAQALQAVISHRAALTQAG

SEQ ID NO: 76 - protein, 33 aa, Burkholderia sp.
FSGGDAVSTVVRSGGAQSVASGGTASGTTVSAG

SEQ ID NO: 77 - protein, 33 aa, Burkholderia sp.
FRQTDIVKMAGSGGSAQALNAVIKHGPTLRQRG

SEQ ID NO: 78 - protein, 33 aa, Burkholderia sp.
FSLIDIVEIASNGGAQALKAVLKYGPVLTQAGR

SEQ ID NO: 79 - protein, 33 aa, Burkholderia sp.
FSGGDAAGTVVSSGGAQNVTGGLASGTTVASGG

SEQ ID NO: 80 - protein, 33 aa, Paraburkholderia sp.
FNLTDIVEMAANSGGAQALKAVLEHGPTLRQRG

SEQ ID NO: 81 - protein, 33 aa, Paraburkholderia sp.
FNRASIVKIAGNSGGAQALQAVLKHGPTLDERG

SEQ ID NO: 82 - protein, 33 aa, Paraburkholderia sp.
FSQANIVKMAGNSGGAQALQAVLDLELVFRERG

SEQ ID NO: 83 - protein, 33 aa, Paraburkholderia sp.
FSQPDIVKMAGNSGGAQALQAVLDLELAFRERG

SEQ ID NO: 84 - protein, 33 aa, Paraburkholderia sp.
FSLIDIVEIASNGGAQALKAVLKYGPVLMQAGR

SEQ ID NO: 85 - protein, 33 aa, Francisella sp.
YKSEDIIRLASHDGGSVNLEAVLRLHSQLTRLG

SEQ ID NO: 86 - protein, 33 aa, Francisella sp.
YKPEDIIRLASHGGGSVNLEAVLRLNPQLIGLG

SEQ ID NO: 87 - protein, 33 aa, Francisella sp.
YKSEDIIRLASHGGGSVNLEAVLRLHSQLTRLG

SEQ ID NO: 88 - protein, 33 aa, Francisella sp.
YKSEDIIRLASHGGGSVNLEAVLRLNPQLIGLG

SEQ ID NO: 89 - protein, 33 aa, Legionella quateirensis
LGHKELIKIAARNGGGNNLIAVLSCYAKLKEMG

SEQ ID NO: 90 - DNA, 19 bp, synthetic construct
  source 1..19: mol_type = other DNA
gaggaagagggggcgggag

SEQ ID NO: 91 - DNA, 20 bp, synthetic construct
  source 1..20: mol_type = other DNA
gagggggcgggagcaagggg

SEQ ID NO: 92 - DNA, 19 bp, synthetic construct
  source 1..19: mol_type = other DNA
gagcaaggggcgggcaccc

SEQ ID NO: 93 - DNA, 20 bp, synthetic construct
  source 1..20: mol_type = other DNA
gaagggagggtgcccgcccc

SEQ ID NO: 94 - DNA, 20 bp, synthetic construct
  source 1..20: mol_type = other DNA
gacctgggacagtttccctt

SEQ ID NO: 95 - DNA, 19 bp, synthetic construct
  source 1..19: mol_type = other DNA
gacagtttcccttccgctc

SEQ ID NO: 96 - DNA, 20 bp, synthetic construct
  source 1..20: mol_type = other DNA
gagcagccccaccagagtgc

SEQ ID NO: 97 - DNA, 20 bp, synthetic construct
  source 1..20: mol_type = other DNA
gatctgcatgcctggagcag

SEQ ID NO: 98 - protein, 932 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFNVEQIVRMVSHNGGSKNLKAVTDNHDDLKNMGFNVEQIVRMVSHNGGSKNLKAVTDNHDDLKNMGFSSQQIIRMVSHAGGANNLKAVTANHDDLQNMGFSSQQIIRMVSHAGGANNLKAVTANHDDLQNMGFNVEQIVRMVSHNGGSKNLKAVTDNHDDLKNMGFSSQQIIRMVSHAGGANNLKAVTANHDDLQNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNTEQIVRMVSHDGGSLNLKAVKKYHDALRERKFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFSSQQIIRMVSHAGGANNLKAVTANHDDLQNMGFNAEQIVRMVSHKGGSKNLALVKEYFPVFSSFHFTADQIVALICQSKQCFRNLKKNHQQWKNKGLSAEQIVDLILQETPPKPNFNNTSSSTPSPSAPSFFQGPSTPIPTPVLDNSPAPIFSNPVCFFSSRSENNTEQYLQDSTLDLDSQLGDPTKNFNVNNFWSLFPFDDVGYHPHSNDVGYHLHSDEESPFFDF

SEQ ID NO: 99 - protein, 819 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFNVEQIVRMVSHNGGSKNLKAVTDNHDDLKNMGFNVEQIVRMVSHNGGSKNLKAVTDNHDDLKNMGFSSQQIIRMVSHAGGANNLKAVTANHDDLQNMGFSSQQIIRMVSHAGGANNLKAVTANHDDLQNMGFNVEQIVRMVSHNGGSKNLKAVTDNHDDLKNMGFSSQQIIRMVSHAGGANNLKAVTANHDDLQNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNTEQIVRMVSHDGGSLNLKAVKKYHDALRERKFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFSSQQIIRMVSHAGGANNLKAVTANHDDLQNMGFNAEQIVRMVSHKGGSKNLALVKEYFPVFSSFHFTADQIVALICQSKQCFRNLKKNHQQWKNKGLSAEQIVDLILQETPPKP

SEQ ID NO: 100 - protein, 965 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNTEQIVRMVSHDGGSLNLKAVKKYHDALRERKFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFSSQQIIRMVSHAGGANNLKAVTANHDDLQNMGFNVEQIVRMVSHNGGSKNLKAVTDNHDDLKNMGFNTEQIVRMVSHDGGSLNLKAVKKYHDALRERKFSSQQIIRMVSHAGGANNLKAVTANHDDLQNMGFSSQQIIRMVSHAGGANNLKAVTANHDDLQNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNAEQIVRMVSHKGGSKNLALVKEYFPVFSSFHFTADQIVALICQSKQCFRNLKKNHQQWKNKGLSAEQIVDLILQETPPKPNFNNTSSSTPSPSAPSFFQGPSTPIPTPVLDNSPAPIFSNPVCFFSSRSENNTEQYLQDSTLDLDSQLGDPTKNFNVNNFWSLFPFDDVGYHPHSNDVGYHLHSDEESPFFDF

SEQ ID NO: 101 - protein, 852 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNTEQIVRMVSHDGGSLNLKAVKKYHDALRERKFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFSSQQIIRMVSHAGGANNLKAVTANHDDLQNMGFNVEQIVRMVSHNGGSKNLKAVTDNHDDLKNMGFNTEQIVRMVSHDGGSLNLKAVKKYHDALRERKFSSQQIIRMVSHAGGANNLKAVTANHDDLQNMGFSSQQIIRMVSHAGGANNLKAVTANHDDLQNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNAEQIVRMVSHKGGSKNLALVKEYFPVFSSFHFTADQIVALICQSKQCFRNLKKNHQQWKNKGLSAEQIVDLILQETPPKP

SEQ ID NO: 102 - protein, 932 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFNVEQIVRMVSHNGGSKNLKAVTDNHDDLKNMGFNTEQIVRMVSHDGGSLNLKAVKKYHDALRERKFSSQQIIRMVSHAGGANNLKAVTANHDDLQNMGFSSQQIIRMVSHAGGANNLKAVTANHDDLQNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNTEQIVRMVSHDGGSLNLKAVKKYHDALRERKFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNTEQIVRMVSHDGGSLNLKAVKKYHDALRERKFSSQQIIRMVSHAGGANNLKAVTANHDDLQNMGFNTEQIVRMVSHDGGSLNLKAVKKYHDALRERKFNTEQIVRMVSHDGGSLNLKAVKKYHDALRERKFNAEQIVRMVSHDGGSKNLALVKEYFPVFSSFHFTADQIVALICQSKQCFRNLKKNHQQWKNKGLSAEQIVDLILQETPPKPNFNNTSSSTPSPSAPSFFQGPSTPIPTPVLDNSPAPIFSNPVCFFSSRSENNTEQYLQDSTLDLDSQLGDPTKNFNVNNFWSLFPFDDVGYHPHSNDVGYHLHSDEESPFFDF

SEQ ID NO: 103 - protein, 819 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFNVEQIVRMVSHNGGSKNLKAVTDNHDDLKNMGFNTEQIVRMVSHDGGSLNLKAVKKYHDALRERKFSSQQIIRMVSHAGGANNLKAVTANHDDLQNMGFSSQQIIRMVSHAGGANNLKAVTANHDDLQNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNTEQIVRMVSHDGGSLNLKAVKKYHDALRERKFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNTEQIVRMVSHDGGSLNLKAVKKYHDALRERKFSSQQIIRMVSHAGGANNLKAVTANHDDLQNMGFNTEQIVRMVSHDGGSLNLKAVKKYHDALRERKFNTEQIVRMVSHDGGSLNLKAVKKYHDALRERKFNAEQIVRMVSHDGGSKNLALVKEYFPVFSSFHFTADQIVALICQSKQCFRNLKKNHQQWKNKGLSAEQIVDLILQETPPKP

SEQ ID NO: 104 - protein, 965 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFSSQQIIRMVSHAGGANNLKAVTANHDDLQNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFSSQQIIRMVSHAGGANNLKAVTANHDDLQNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNAEQIVSMVSNGGGSLNLKAVKKYHDALKDRGFNVEQIVRMVSHNGGSKNLKAVTDNHDDLKNMGFNTEQIVRMVSHDGGSLNLKAVKKYHDALRERKFNTEQIVRMVSHDGGSLNLKAVKKYHDALRERKFNTEQIVRMVSHDGGSLNLKAVKKYHDALRERKFNVEQIVRMVSHNGGSKNLKAVTDNHDDLKNMGFNTEQIVRMVSHDGGSLNLKAVKKYHDALRERKFNTEQIVRMVSHDGGSLNLKAVKKYHDALRERKFNTEQIVRMVSHDGGSLNLKAVKKYHDALRERKFNAEQIVRMVSHDGGSKNLALVKEYFPVFSSFHFTADQIVALICQSKQCFRNLKKNHQQWKNKGLSAEQIVDLILQETPPKPNFNNTSSSTPSPSAPSFFQGPSTPIPTPVLDNSPAPIFSNPVCFFSSRSENNTEQYLQDSTLDLDSQLGDPTKNFNVNNFWSLFPFDDVGYHPHSNDVGYHLHSDEESPFFDF

SEQ ID NO: 105 - protein, 852 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFSSQQIIRMVSHAGGANNLKAVTANHDDLQNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFSSQQIIRMVSHAGGANNLKAVTANHDDLQNMGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFNAEQIVSMVSNGGGSLNLKAVKKYHDALKDRGFNVEQIVRMVSHNGGSKNLKAVTDNHDDLKNMGFNTEQIVRMVSHDGGSLNLKAVKKYHDALRERKFNTEQIVRMVSHDGGSLNLKAVKKYHDALRERKFNTEQIVRMVSHDGGSLNLKAVKKYHDALRERKFNVEQIVRMVSHNGGSKNLKAVTDNHDDLKNMGFNTEQIVRMVSHDGGSLNLKAVKKYHDALRERKFNTEQIVRMVSHDGGSLNLKAVKKYHDALRERKFNTEQIVRMVSHDGGSLNLKAVKKYHDALRERKFNAEQIVRMVSHDGGSKNLALVKEYFPVFSSFHFTADQIVALICQSKQCFRNLKKNHQQWKNKGLSAEQIVDLILQETPPKP

SEQ ID NO: 106 - protein, 965 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFNTEQIVRMVSHDGGSLNLKAVKKYHDALRERKFNTEQIVRMVSHDGGSLNLKAVKKYHDALRERKFNAEQIVSMVSNGGGSLNLKAVKKYHDALKDRGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFSSQQIIRMVSHAGGANNLKAVTANHDDLQNMGFNTEQIVRMVSHDGGSLNLKAVKKYHDALRERKFSSQQIIRMVSHAGGANNLKAVTANHDDLQNMGFNVEQIVRMVSHNGGSKNLKAVTDNHDDLKNMGFNAEQIVSMVSNGGGSLNLKAVKKYHDALKDRGFNAEQIVSMVSNGGGSLNLKAVKKYHDALKDRGFNAEQIVSMVSNGGGSLNLKAVKKYHDALKDRGFNTEQIVRMVSHDGGSLNLKAVKKYHDALRERKFNTEQIVRMVSHDGGSLNLKAVKKYHDALRERKFNTEQIVRMVSHDGGSLNLKAVKKYHDALRERKFNAEQIVSMVSNGGGSLNLKAVKKYHDALKDRGFNAEQIVRMVSNGGGSKNLALVKEYFPVFSSFHFTADQIVALICQSKQCFRNLKKNHQQWKNKGLSAEQIVDLILQETPPKPNFNNTSSSTPSPSAPSFFQGPSTPIPTPVLDNSPAPIFSNPVCFFSSRSENNTEQYLQDSTLDLDSQLGDPTKNFNVNNFWSLFPFDDVGYHPHSNDVGYHLHSDEESPFFDF

SEQ ID NO: 107 - protein, 852 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFNTEQIVRMVSHDGGSLNLKAVKKYHDALRERKFNTEQIVRMVSHDGGSLNLKAVKKYHDALRERKFNAEQIVSMVSNGGGSLNLKAVKKYHDALKDRGFNVEQIVRMVSHNGGSKNLKAVTDNHDDLKNMGFNVEQIVRMVSHNGGSKNLKAVTDNHDDLKNMGFNVEQIVRMVSHNGGSKNLKAVTDNHDDLKNMGFSSQQIIRMVSHAGGANNLKAVTANHDDLQNMGFNTEQIVRMVSHDGGSLNLKAVKKYHDALRERKFSSQQIIRMVSHAGGANNLKAVTANHDDLQNMGFNVEQIVRMVSHNGGSKNLKAVTDNHDDLKNMGFNAEQIVSMVSNGGGSLNLKAVKKYHDALKDRGFNAEQIVSMVSNGGGSLNLKAVKKYHDALKDRGFNAEQIVSMVSNGGGSLNLKAVKKYHDALKDRGFNTEQIVRMVSHDGGSLNLKAVKKYHDALRERKFNTEQIVRMVSHDGGSLNLKAVKKYHDALRERKFNTEQIVRMVSHDGGSLNLKAVKKYHDALRERKFNAEQIVSMVSNGGGSLNLKAVKKYHDALKDRGFNAEQIVRMVSNGGGSKNLALVKEYFPVFSSFHFTADQIVALICQSKQCFRNLKKNHQQWKNKGLSAEQIVDLILQETPPKP

SEQ ID NO: 108 - protein, 932 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFNTEQIVRMVSHDGGSLNLKAVKKYHDALRERKFSSQQIIRMVSHAGGANNLKAVTANHDDLQNMGFNVEQIVRMVSHNGGSKNLKAVTDNHDDLKNMGFNAEQIVSMVSNGGGSLNLKAVKKYHDALKDRGFNAEQIVSMVSNGGGSLNLKAVKKYHDALKDRGFNAEQIVSMVSNGGGSLNLKAVKKYHDALKDRGFNTEQIVRMVSHDGGSLNLKAVKKYHDALRERKFNTEQIVRMVSHDGGSLNLKAVKKYHDALRERKFNTEQIVRMVSHDGGSLNLKAVKKYHDALRERKFNAEQIVSMVSNGGGSLNLKAVKKYHDALKDRGFNAEQIVSMVSNGGGSLNLKAVKKYHDALKDRGFNTEQIVRMVSHDGGSLNLKAVKKYHDALRERKFNTEQIVRMVSHDGGSLNLKAVKKYHDALRERKFNVEQIVRMVSHNGGSKNLKAVTDNHDDLKNMGFNTEQIVRMVSHDGGSLNLKAVKKYHDALRERKFNAEQIVSMVSNGGGSLNLKAVKKYHDALKDRGFNAEQIVRMVSHDGGSKNLALVKEYFPVFSSFHFTADQIVALICQSKQCFRNLKKNHQQWKNKGLSAEQIVDLILQETPPKPNFNNTSSSTPSPSAPSFFQGPSTPIPTPVLDNSPAPIFSNPVCFFSSRSENNTEQYLQDSTLDLDSQLGDPTKNFNVNNFWSLFPFDDVGYHPHSNDVGYHLHSDEESPFFDF

SEQ ID NO: 109 - protein, 819 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFNTEQIVRMVSHDGGSLNLKAVKKYHDALRERKFSSQQIIRMVSHAGGANNLKAVTANHDDLQNMGFNVEQIVRMVSHNGGSKNLKAVTDNHDDLKNMGFNAEQIVSMVSNGGGSLNLKAVKKYHDALKDRGFNAEQIVSMVSNGGGSLNLKAVKKYHDALKDRGFNAEQIVSMVSNGGGSLNLKAVKKYHDALKDRGFNTEQIVRMVSHDGGSLNLKAVKKYHDALRERKFNTEQIVRMVSHDGGSLNLKAVKKYHDALRERKFNTEQIVRMVSHDGGSLNLKAVKKYHDALRERKFNAEQIVSMVSNGGGSLNLKAVKKYHDALKDRGFNAEQIVSMVSNGGGSLNLKAVKKYHDALKDRGFNTEQIVRMVSHDGGSLNLKAVKKYHDALRERKFNTEQIVRMVSHDGGSLNLKAVKKYHDALRERKFNVEQIVRMVSHNGGSKNLKAVTDNHDDLKNMGFNTEQIVRMVSHDGGSLNLKAVKKYHDALRERKFNAEQIVSMVSNGGGSLNLKAVKKYHDALKDRGFNAEQIVRMVSHDGGSKNLALVKEYFPVFSSFHFTADQIVALICQSKQCFRNLKKNHQQWKNKGLSAEQIVDLILQETPPKP

SEQ ID NO: 110 - protein, 965 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFNVEQIVRMVSHNGGSKNLKAVTDNHDDLKNMGFNTEQIVRMVSHDGGSLNLKAVKKYHDALRERKFSSQQIIRMVSHAGGANNLKAVTANHDDLQNMGFNVEQIVRMVSHNGGSKNLKAVTDNHDDLKNMGFNTEQIVRMVSHDGGSLNLKAVKKYHDALRERKFNTEQIVRMVSHDGGSLNLKAVKKYHDALRERKFNTEQIVRMVSHDGGSLNLKAVKKYHDALRERKFNTEQIVRMVSHDGGSLNLKAVKKYHDALRERKFSSQQIIRMVSHAGGANNLKAVTANHDDLQNMGFNTEQIVRMVSHDGGSLNLKAVKKYHDALRERKFNTEQIVRMVSHDGGSLNLKAVKKYHDALRERKFSSQQIIRMVSHAGGANNLKAVTANHDDLQNMGFNVEQIVRMVSHNGGSKNLKAVTDNHDDLKNMGFSSQQIIRMVSHAGGANNLKAVTANHDDLQNMGFNVEQIVRMVSHNGGSKNLKAVTDNHDDLKNMGFNAEQIVSMVSNGGGSLNLKAVKKYHDALKDRGFNVEQIVRMVSHNGGSKNLKAVTDNHDDLKNMGFNAEQIVRMVSHDGGSKNLALVKEYFPVFSSFHFTADQIVALICQSKQCFRNLKKNHQQWKNKGLSAEQIVDLILQETPPKPNFNNTSSSTPSPSAPSFFQGPSTPIPTPVLDNSPAPIFSNPVCFFSSRSENNTEQYLQDSTLDLDSQLGDPTKNFNVNNFWSLFPFDDVGYHPHSNDVGYHLHSDEESPFFDF

SEQ ID NO: 111 - protein, 852 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFNVEQIVRMVSHNGGSKNLKAVTDNHDDLKNMGFNTEQIVRMVSHDGGSLNLKAVKKYHDALRERKFSSQQIIRMVSHAGGANNLKAVTANHDDLQNMGFNVEQIVRMVSHNGGSKNLKAVTDNHDDLKNMGFNTEQIVRMVSHDGGSLNLKAVKKYHDALRERKFNTEQIVRMVSHDGGSLNLKAVKKYHDALRERKFNTEQIVRMVSHDGGSLNLKAVKKYHDALRERKFNTEQIVRMVSHDGGSLNLKAVKKYHDALRERKFSSQQIIRMVSHAGGANNLKAVTANHDDLQNMGFNTEQIVRMVSHDGGSLNLKAVKKYHDALRERKFNTEQIVRMVSHDGGSLNLKAVKKYHDALRERKFSSQQIIRMVSHAGGANNLKAVTANHDDLQNMGFNVEQIVRMVSHNGGSKNLKAVTDNHDDLKNMGFSSQQIIRMVSHAGGANNLKAVTANHDDLQNMGFNVEQIVRMVSHNGGSKNLKAVTDNHDDLKNMGFNAEQIVSMVSNGGGSLNLKAVKKYHDALKDRGFNVEQIVRMVSHNGGSKNLKAVTDNHDDLKNMGFNAEQIVRMVSHDGGSKNLALVKEYFPVFSSFHFTADQIVALICQSKQCFRNLKKNHQQWKNKGLSAEQIVDLILQETPPKP

SEQ ID NO: 112 - protein, 965 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFNAEQIVSMVSNGGGSLNLKAVKKYHDALKDRGFNTEQIVRMVSHDGGSLNLKAVKKYHDALRERKFNAEQIVSMVSNGGGSLNLKAVKKYHDALKDRGFNVEQIVRMVSHNGGSKNLKAVTDNHDDLKNMGFNTEQIVRMVSHDGGSLNLKAVKKYHDALRERKFSSQQIIRMVSHAGGANNLKAVTANHDDLQNMGFNAEQIVSMVSNGGGSLNLKAVKKYHDALKDRGFNVEQIVRMVSHNGGSKNLKAVTDNHDDLKNMGFNTEQIVRMVSHDGGSLNLKAVKKYHDALRERKFNTEQIVRMVSHDGGSLNLKAVKKYHDALRERKFNAEQIVSMVSNGGGSLNLKAVKKYHDALKDRGFNVEQIVRMVSHNGGSKNLKAVTDNHDDLKNMGFNVEQIVRMVSHNGGSKNLKAVTDNHDDLKNMGFSSQQIIRMVSHAGGANNLKAVTANHDDLQNMGFNVEQIVRMVSHNGGSKNLKAVTDNHDDLKNMGFNTEQIVRMVSHDGGSLNLKAVKKYHDALRERKFSSQQIIRMVSHAGGANNLKAVTANHDDLQNMGFNAEQIVRMVSHKGGSKNLALVKEYFPVFSSFHFTADQIVALICQSKQCFRNLKKNHQQWKNKGLSAEQIVDLILQETPPKPNFNNTSSSTPSPSAPSFFQGPSTPIPTPVLDNSPAPIFSNPVCFFSSRSENNTEQYLQDSTLDLDSQLGDPTKNFNVNNFWSLFPFDDVGYHPHSNDVGYHLHSDEESPFFDF

SEQ ID NO: 113 - protein, 852 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFNAEQIVSMVSNGGGSLNLKAVKKYHDALKDRGFNTEQIVRMVSHDGGSLNLKAVKKYHDALRERKFNAEQIVSMVSNGGGSLNLKAVKKYHDALKDRGFNVEQIVRMVSHNGGSKNLKAVTDNHDDLKNMGFNTEQIVRMVSHDGGSLNLKAVKKYHDALRERKFSSQQIIRMVSHAGGANNLKAVTANHDDLQNMGFNAEQIVSMVSNGGGSLNLKAVKKYHDALKDRGFNVEQIVRMVSHNGGSKNLKAVTDNHDDLKNMGFNTEQIVRMVSHDGGSLNLKAVKKYHDALRERKFNTEQIVRMVSHDGGSLNLKAVKKYHDALRERKFNAEQIVSMVSNGGGSLNLKAVKKYHDALKDRGFNVEQIVRMVSHNGGSKNLKAVTDNHDDLKNMGFNVEQIVRMVSHNGGSKNLKAVTDNHDDLKNMGFSSQQIIRMVSHAGGANNLKAVTANHDDLQNMGFNVEQIVRMVSHNGGSKNLKAVTDNHDDLKNMGFNTEQIVRMVSHDGGSLNLKAVKKYHDALRERKFSSQQIIRMVSHAGGANNLKAVTANHDDLQNMGFNAEQIVRMVSHKGGSKNLALVKEYFPVFSSFHFTADQIVALICQSKQCFRNLKKNHQQWKNKGLSAEQIVDLILQETPPKP

SEQ ID NO: 114 - protein, 33 aa, Paraburkholderia sp.
FNLTDIVEMAGKGGGAQALKAVLEHGPTLRQRG

SEQ ID NO: 115 - protein, 33 aa, Paraburkholderia sp.
FRQADIIKIAGNDGGAQALQAVIEHGPTLRQHG

SEQ ID NO: 116 - protein, 33 aa, Paraburkholderia sp.
FSQADIVKIAGNDGGTQALHAVLDLERMLGERG

SEQ ID NO: 117 - protein, 33 aa, Paraburkholderia sp.
FSRADIVKIAGNGGGAQALKAVLEHEATLDERG

SEQ ID NO: 118 - protein, 33 aa, Paraburkholderia sp.
FSRADIVRIAGNGGGAQALYSVLDVEPTLGKRG

SEQ ID NO: 119 - protein, 33 aa, Paraburkholderia sp.
FSQPDIVKMASNIGGAQALQAVLELEPALRERG

SEQ ID NO: 120 - protein, 33 aa, Paraburkholderia sp.
FSQPDIVKMAGNIGGAQALQAVLSLGPALRERG

SEQ ID NO: 121 - protein, 33 aa, Paraburkholderia sp.
FSQPEIVKIAGNIGGAQALHTVLELEPTLHKRG

SEQ ID NO: 122 - protein, 33 aa, Paraburkholderia sp.
FSQSDIVKIAGNIGGAQALQAVLDLESMLGKRG

SEQ ID NO: 123 - protein, 33 aa, Paraburkholderia sp.
FSQSDIVKIAGNIGGAQALQAVLELEPTLRESD

SEQ ID NO: 124 - protein, 33 aa, Paraburkholderia sp.
FNPTDIVKIAGNKGGAQALQAVLELEPALRERG

SEQ ID NO: 125 - protein, 33 aa, Paraburkholderia sp.
FSPTDIIKIAGNNGGAQALQAVLDLELMLRERG

SEQ ID NO: 126 - protein, 33 aa, Paraburkholderia sp.
FSQADIVKIAGNNGGAQALYSVLDVEPTLGKRG

SEQ ID NO: 127 - protein, 33 aa, Paraburkholderia sp.
FSRGDIVTIAGNNGGAQALQAVLELEPTLRERG

SEQ ID NO: 128 - protein, 33 aa, Paraburkholderia sp.
FSRIDIVKIAANNGGAQALHAVLDLGPTLRECG

SEQ ID NO: 129 - protein, 33 aa, Paraburkholderia sp.
FSQADIVKIVGNNGGAQALQAVFELEPTLRERG

SEQ ID NO: 130 - protein, 33 aa, Paraburkholderia sp.
FSQPDIVRITGNRGGAQALQAVLALELTLRERG

SEQ ID NO: 131 - protein, 33 aa, Legionellales bacterium RIFCSPHIGHO2_12_FULL_35_11
FKADDAVRIACRTGGSHNLKAVHKNYERLRARG

SEQ ID NO: 132 - protein, 33 aa, Legionellales bacterium RIFCSPHIGHO2_12_FULL_35_11
FNADQVIKIVGHDGGSNNIDVVQQFFPELKAFG

SEQ ID NO: 133 - protein, 33 aa, Legionella maceachernii
FSAEQIVRIAAHIGGSRNIEATIKHYAMLTQPP

SEQ ID NO: 134 - protein, 33 aa, Francisella sp.
YKSEDIIRLASHDGGSVNLEAVLRLNPQLIGLG

SEQ ID NO: 135 - protein, 33 aa, Francisella sp.
YKSEDIIRLASHDGGSINLEAVLRLNPQLIGLG

SEQ ID NO: 136 - protein, 33 aa, Francisella sp.
YKSEDIIRLASSNGGSVNLEAVLRLNPQLIGLG

SEQ ID NO: 137 - protein, 33 aa, Francisella sp.
YKSEDIIRLASSNGGSVNLEAVIAVHKALHSNG

SEQ ID NO: 138 - protein, 33 aa, Legionellales bacterium RIFCSPHIGHO2_12_FULL_35_11
FSADQVVKIAGHSGGSNNIAVMLAVFPRLRDFG

SEQ ID NO: 139 - protein, 365 aa, Legionellales bacterium RIFCSPHIGHO2_12_FULL_35_11
MPKTKITTVSHGYDLDLMSSLPNGDPNQAKQGKIYLSGNGVYVVRDVAGIVHRGQLEFAINLEQLEQKINEPAFKAVILEKTSRAVGYTISNECFNVELNALAKAGFNNLDIDKLIFRRSSRGTVQTVLNSYNILLEKPYNLDRQQILRIASHDGGSKNIAAVQKFLPKLMNFGFNADQVIKIVGHDGGSNNIDVVQQFFPELKAFGFSADQVVKIAGHSGGSNNIAVMLAVFPRLRDFGFKADDAVRIACRTGGSHNLKAVHKNYERLRARGYDNKKIISIAASNCGTETINTIMSTDEVEESDFLYFVTTVSTPVASQNLSSASNTNINYSNRFMTARKKTSDDNTDEVEEDQHRDKRRSNGR

SEQ ID NO: 140 - protein, 141 aa, synthetic construct
MPKTKITTVSHGYDLDLMSSLPNGDPNQAKQGKIYLSGNGVYVVRDVAGIVHRGQLEFAINLEQLEQKINEPAFKAVILEKTSRAVGYTISNECFNVELNALAKAGFNNLDIDKLIFRRSSRGTVQTVLNSYNILLEKPYN

SEQ ID NO: 141 - protein, 92 aa, synthetic construct
YDNKKIISIAASNCGTETINTIMSTDEVEESDFLYFVTTVSTPVASQNLSSASNTNINYSNRFMTARKKTSDDNTDEVEEDQHRDKRRSNGR

SEQ ID NO: 142 - protein, 63 aa, synthetic construct
YDNKKIISIAASNCGTETINTIMSTDEVEESDFLYFVTTVSTPVASQNLSSASNTNINYSNRF

SEQ ID NO: 143 - protein, 808 aa, Legionella maceachernii
MPKTNQPKNLEAKSTKNKISLPQDPQTLNELKIKGYPQDLAERLIKKGSSLAVKTVLKDHEQLVNFFTHLQIIRMAAQKGGAKNITTALNEYNSLTNLGYSSEQIVRVAAHGGGSLNIKAVLQAHQALKELDFSAEQIVRIAAHDGGSLNIDAVQQAQQALKELGFSAKHIVRIAAHIGGSLNIKAVQQAQQALKELGFSADQIVRIAAHKGGSHNIVAVQQAQQALKELDFSAEQIVRIAAHIGGSRNIEAIQQAHHALKELGFSAEQIVRIAAHIGGSHNLKAVLQAQQALKELDFSAEQIVRIAAHDGGSRNIEAVQQAQHVLKELGFSAEQIVHIAAHGGGSLNIKAILQAHQTLKELNFSAEQIVSIVAHDGGSRNIEAVQQAQHILKELGFSTEQIVCIAGHGGGSLNIKAVLLAQQALKDLGFSAEQIVSIAAHVGGSHNIEAVQKAHQALKELDFSAEQIVRIAAHIGGSRNIEATIKHYAMLTQPPYMLSQEQFLRLIDHHSGHLNLSILLDEQQWQAINDLCLQPHHFGRQNALEKFLQQGQRKYQNLQELEQFLFQDSADPMLLQETENQHEAEKINDCMDFILRLISATEPLDLQIEIEGIGLFSPSMHFDATQANFSTPAANEEKIDNSATEAGVNSRKRKIAAAHQKQPPRKKTATPLSATFISTLTTLAQSDNPRLEMASAEALMLKAPQKLAMGITVRKKTKCEGIAIITVTDKTKLNGWLSSASESTYSSVEAQGTRTVNNTHAFFSTPLTSDKKSPSFSSLDFYEDSGLGFDEEITNPPYMPELEPEFIL

SEQ ID NO: 144 - protein, 99 aa, synthetic construct
MPKTNQPKNLEAKSTKNKISLPQDPQTLNELKIKGYPQDLAERLIKKGSSLAVKTVLKDHEQLVNFFTHLQIIRMAAQKGGAKNITTALNEYNSLTNLG

SEQ ID NO: 145 - protein, 313 aa, synthetic construct
YMLSQEQFLRLIDHHSGHLNLSILLDEQQWQAINDLCLQPHHFGRQNALEKFLQQGQRKYQNLQELEQFLFQDSADPMLLQETENQHEAEKINDCMDFILRLISATEPLDLQIEIEGIGLFSPSMHFDATQANFSTPAANEEKIDNSATEAGVNSRKRKIAAAHQKQPPRKKTATPLSATFISTLTTLAQSDNPRLEMASAEALMLKAPQKLAMGITVRKKTKCEGIAIITVTDKTKLNGWLSSASESTYSSVEAQGTRTVNNTHAFFSTPLTSDKKSPSFSSLDFYEDSGLGFDEEITNPPYMPELEPEFIL

SEQ ID NO: 146 - protein, 63 aa, synthetic construct
YMLSQEQFLRLIDHHSGHLNLSILLDEQQWQAINDLCLQPHHFGRQNALEKFLQQGQRKYQNL

SEQ ID NO: 147 - protein, 1356 aa, Francisella sp.
MTLTLKQEKIAINKKLRSYRTSKRKFLLDFSKMNLSPEGLNYAQEELAKLQFQAKASLETDQGINIEEQLYQLGYTQSHLRPCADRYNCSILLNTLLTNNNSFVTQEISLENRVNLVVAANGNNDGIQVFFKTYPKLKSVGYKINHCVNLLKLNHDGFMLKNLIPYDSKLTGLGYKPEDIIRLASHGGGSVNLEAVLRLNPQLIGLGYKSEDIIRLASHDGGSVNLEAVLRLHSQLTRLGYKSEDIIRLASHGGGSVNLEAVLRLHSQLTRLGYKSEDIIRLASHGGGSVNLEAVLRLNPQLIGLGYKSEDIIRLASHGGGSVNLEAVLRLHSQLTRLGYKSEDIIRLASHGGGSVNLEAVLRLNPQLIGLGYKSEDIIRLASHGGGSVNLEAVLRLHSQLTRLGYKSEDIIRLASHGGGSVNLEAVLRLNPQLIGLGYKSEDIIRLASHDGGSVNLEAVLRLNPQLIGLGYKSEDIIRLASHDGGSINLEAVLRLNPQLIGLGYKSEDIIRLASSNGGSVNLEAVLRLNPQLIGLGYKSEDIIRLASSNGGSVNLEAVLRLNPQLIGLGYKSEDIIRLASHDGGSVNLEAVLRLHSQLTRLGYKSEDIIRLASSNGGSVNLEAVIAVHKALHSNGYNKKQIVLIASGSSGGMRLHALNNYHFLFHSIENIQLLITILQSHLEAFRIEQYMISGVLLNLLKQGQVISEQPCKILIDSSILNPNICQTLSNIINKYQFKNKPLYFNPTTSIITCMLSTQECYQLLAVWERRNISPSEILNNLLNPINIFQYQLISQTNEPDVYFLDCYHWHKFYPNMEIKQLQQLLIKAINLGINNCDILPEDNRTLIIEPYNDNWIKLSISIIDTIMDDSFNNLTRELFFCQLAPDSSNLIDDAIYIYKTQQTIEFLVTSKSRSSERFILDTSTIYKDTIEEIEQALTHKLGALKGATYHTLIKCLLAQGYQVTGYFSMNIIGADVMPPTIIADDYPEYITLEWLSSEPMSQRSRLRTHDINSIKTLHNPTPKSQAIHQMLNLLALPDAISPLDSIQNNHTSANHEQQTQGRISPISQQLDITLMRSRKRPLQKSDNTIYHDKRYWTFIGEGSYNKAYTDGQGFVVKVAKNELGLMDKSERSVRVFNEINPTLPQEVLAHVSQDLWISPLIENETLSPIEQASFIFKTYIEHGRLILDGYCQNNLLQSAKYNTPVCIDPGNVVRRNSIASQEHWYAANEKTLLRRQLYRKHMIDTIDHYHKIRHIDRTLPILMILALDFIDRKMQHLQLQLILKKNIKSLGIAFYFYYKHNQSSTQQEFILSANIIDKILYGDQYICDTLDQSFKTLNKSRVVTLFRQINIDMSLI

SEQ ID NO: 148 - protein, 141 aa, synthetic construct
MTLTLKQEKIAINKKLRSYRTSKRKFLLDFSKMNLSPEGLNYAQEELAKLQFQAKASLETDQGINIEEQLYQLGYTQSHLRPCADRYNCSILLNTLLTNNNSFVTQEISLENRVNLVVAANGNNDGIQVFFKTYPKLKSVG

SEQ ID NO: 149 - protein, 704 aa, synthetic construct
MRLHALNNYHFLFHSIENIQLLITILQSHLEAFRIEQYMISGVLLNLLKQGQVISEQPCKILIDSSILNPNICQTLSNIINKYQFKNKPLYFNPTTSIITCMLSTQECYQLLAVWERRNISPSEILNNLLNPINIFQYQLISQTNEPDVYFLDCYHWHKFYPNMEIKQLQQLLIKAINLGINNCDILPEDNRTLIIEPYNDNWIKLSISIIDTIMDDSFNNLTRELFFCQLAPDSSNLIDDAIYIYKTQQTIEFLVTSKSRSSERFILDTSTIYKDTIEEIEQALTHKLGALKGATYHTLIKCLLAQGYQVTGYFSMNIIGADVMPPTIIADDYPEYITLEWLSSEPMSQRSRLRTHDINSIKTLHNPTPKSQAIHQMLNLLALPDAISPLDSIQNNHTSANHEQQTQGRISPISQQLDITLMRSRKRPLQKSDNTIYHDKRYWTFIGEGSYNKAYTDGQGFVVKVAKNELGLMDKSERSVRVFNEINPTLPQEVLAHVSQDLWISPLIENETLSPIEQASFIFKTYIEHGRLILDGYCQNNLLQSAKYNTPVCIDPGNVVRRNSIASQEHWYAANEKTLLRRQLYRKHMIDTIDHYHKIRHIDRTLPILMILALDFIDRKMQHLQLQLILKKNIKSLGIAFYFYYKHNQSSTQQEFILSANIIDKILYGDQYICDTLDQSFKTLNKSRVVTLFRQINIDMSLI

SEQ ID NO: 150 - protein, 47 aa, synthetic construct
MRLHALNNYHFLFHSIENIQLLITILQSHLEAFRIEQYMISGVLLNL

SEQ ID NO: 151 - protein, 33 aa, Francisella sp.
YKINHCVNLLKLNHDGFMLKNLIPYDSKLTGLG

SEQ ID NO: 152 - protein, 16 aa, Francisella sp.
YNKKQIVLIASGSSGG

SEQ ID NO: 153-154 - skipped (2 entries)

SEQ ID NO: 155 - protein, 19 aa, synthetic construct
  VARIANT 12..13: note = The amino acid sequence from position 12 to 13 may be HK, HD, HA, HN, HG, NN, NG, RN, HI, HV, RT, HD, SN, HS, GS, or LN.
FNAEQIVRMVSXXGGSKNL

SEQ ID NO: 156 - protein, 33 aa, synthetic construct
  VARIANT 3: note = The amino acid at position 3 may be P or S.
  VARIANT 13: note = The amino acid at position 13 may be D or G.
  VARIANT 26: note = The amino acid at position 26 may be H or N.
  VARIANT 27: note = The amino acid at position 27 may be P or S.
  VARIANT 30: note = The amino acid at position 30 may be I ro T.
  VARIANT 31: note = The amino acid at position 31 may be G or R.
YKXEDIIRLASHXGGSVNLEAVLRLXXQLXXLG

SEQ ID NO: 157 - skipped

SEQ ID NO: 158 - protein, 33 aa, synthetic construct
  VARIANT 1: note = The amino acid at position 1 may be F or L.
  VARIANT 2: note = The amino acid at position 2 may be N, S, or G.
  VARIANT 3: note = The amino acid at position 3 may be S, V, A, T, or H.
  VARIANT 4: note = The amino acid at position 4 may be E, Q, or K.
  VARIANT 5: note = The amino acid at position 5 may be Q or E.
  VARIANT 6: note = The amino acid at position 6 may be I or L.
  VARIANT 7: note = The amino acid at position 7 may be I or V.
  VARIANT 8: note = The amino acid at position 8 may be R, S, or K.
  VARIANT 9: note = The amino acid at position 9 may be M or I.
  VARIANT 10: note = The amino acid at position 10 may be V or A.
  VARIANT 11: note = The amino acid at position 12 may be S or A.
  VARIANT 12..13: note = The amino acid sequence at positions 12 to 13 may be HK, HD, HA, HN, HG, NN, NG, RN, HI, HV, RT, HD, SN, HS, GS, or LN.
  VARIANT 16: note = The amino acid at position 16 may be G, A, or S.
  VARIANT 17: note = The amino acid at position 17 may be L, K, or N.
  VARIANT 20: note = The amino acid at position 20 may be K or I.
  VARIANT 23: note = The amino acid at position 23 may be T, K, or L.
  VARIANT 24: note = The amino acid at position 24 may be A, D, K, or S.
  VARIANT 25: note = The amino acid at position 25 may be N, Y, or C.
  VARIANT 26: note = The amino acid at position 26 may be H or Y.
  VARIANT 27: note = The amino acid at position 27 may be D or K.
  VARIANT 28: note = The amino acid at position 28 may be D, A, or V.
  VARIANT 30: note = The amino acid at position 30 may be Q, K, or R.
  VARIANT 31: note = The amino acid at position 31 may be N, D, or E.
  VARIANT 32: note = The amino acid at position 32 may be M or R.
  VARIANT 33: note = The amino acid at position 33 may be G, K, or E.
XXXXXXXXXXXXXGGXXNLXAVXXXXXXLXXXX

SEQ ID NO: 159 - protein, 162 aa, synthetic construct
FTADQIVALICQSKQCFRNLKKNHQQWKNKGLSAEQIVDLILQETPPKPNFNNTSSSTPSPSAPSFFQGPSTPIPTPVLDNSPAPIFSNPVCFFSSRSENNTEQYLQDSTLDLDSQLGDPTKNFNVNNFWSLFPFDDVGYHPHSNDVGYHLHSDEESPFFDF

(19) United States
(12) Patent Application Publication
(10) Pub. No.: US 2025/0361275 A1
(43) Pub. Date: Nov. 27, 2025

(54) Animal Pathogen-Derived Polypeptides and Uses Thereof for Genetic Engineering

(71) Applicant: Altius Institute for Biomedical Sciences, Seattle, WA (US)

(72) Inventors: Fyodor Urnov, Seattle, WA (US); John A. Stamatoyannopoulos, Seattle, WA (US); Alister PW Funnell, Seattle, WA (US)

(21) Appl. No.: 19/193,869

(22) Filed: Apr. 29, 2025

Related U.S. Application Data
(62) Division of application No. 17/047,373, filed on Oct. 13, 2020, now Pat. No. 12,312,383.
(60) Provisional application No. 62/659,656, filed on Apr. 18, 2018.
(60) Provisional application No. 62/690,905, filed on Jun. 27, 2018.
(60) Provisional application No. 62/716,223, filed on Aug. 8, 2018.
(60) Provisional application No. 62/738,825, filed on Sep. 28, 2018.
(60) Provisional application No. 62/819,237, filed on Mar. 15, 2019.

(51) Int. Cl. C07K 14/195

(52) U.S. Cl. CPC C07K 14/195; C07K 2319/80

(57) ABSTRACT

Provided herein are polypeptides, compositions, and methods for use thereof for genetic and epigenomic engineering, including, genome editing and gene regulation. These polypeptides and compositions include nucleic acid binding domains that bind to a target nucleic acid of interest. The nucleic acid binding domains include repeat units derived from repeat units identified in proteins from animal pathogens such as bacterium of the order Legionellales and the species Legionella and Francisella.

### CROSS-REFERENCE TO RELATED APPLICATIONS

This application is a divisional of U.S. application Ser. No. 17/047,373, filed on Oct. 13, 2020, which application is a U.S. National Stage of International Application No. PCT/US2019/028174, filed on Apr. 18, 2019, which claims the benefit of U.S. Provisional Application No. 62/659,656, filed on Apr. 18, 2018, U.S. Provisional Application No. 62/690,905, filed on Jun. 27, 2018, U.S. Provisional Application No. 62/716,223, filed on Aug. 8, 2018, U.S. Provisional Application No. 62/738,825, filed on Sep. 28, 2018, and U.S. Provisional Application No. 62/819,237, filed on Mar. 15, 2019, the disclosures of which are incorporated herein by reference in its entirety.

### REFERENCE TO AN ELECTRONIC SEQUENCE LISTING

The contents of the electronic sequence listing (ALTI-720DIV_SEQ_LIST.xml; Size: 1,787,242 bytes; and date of creation: Apr. 15, 2025) is herein incorporated in its entirety.

### INTRODUCTION

Genome editing and gene regulation techniques include the use of nucleic acid binding domains that bind to a target nucleic acid. Nucleic acid binding domains include RNA-guided domains as used in CRISPR-Cas9 mediated-gene editing and protein-only domains such as zinc-finger proteins, TALE proteins, and meganucleases. Due to the importance of genome engineering in applications in a wide variety of areas, including therapeutics, there is a need for nucleic acid binding domains that have desirable features such as ease of production, target specificity, and versatility.

### SUMMARY

Provided herein are polypeptides, compositions thereof, and methods for genetic and epigenomic engineering, including, genome editing and gene regulation using the polypeptides and compositions, where polypeptides include a nucleic acid binding domain derived from nucleic acid binding proteins identified in animal pathogens, such as a bacterium from the order Legionellales or the genus Legionella or Francisella.

In various aspects, the present disclosure provides a composition comprising a non-naturally occurring modular nucleic acid binding domain derived from an animal pathogen protein (MAP-NBD), wherein the MAP-NBD comprises a plurality of repeat units and wherein a repeat unit (RU) of the plurality of repeat units (RUs) recognizes a base in a target nucleic acid. In some aspects, the animal pathogen protein is derived from a bacterium that infects animals. In some aspects, the animal pathogen protein is derived from a bacterium that infects humans. In some aspects, the bacterium is selected from the order Legionellales or the genus of Legionella or Francisella. In certain aspects, the bacterium is Legionella quateirensis (L. quateirensis) or Legionella maceachernii (L. maceachernii). In some aspects, the repeat unit comprises a consensus sequence of 1xxx211x1xxx33x2x1xxxxxxxxx1.

In some aspects, the target nucleic acid is a single nucleotide or a single base pair. In some aspects, the target nucleic acid is DNA or RNA. In some aspects, the NBD includes at least three RUs, wherein each RU binds to a base in the target nucleic acid and wherein the target nucleic acid is at least three nucleotides in length. In further aspects, the target nucleic acid sequence is DNA or RNA that is at least three nucleotides in length.

In certain aspects, the present disclosure provides a recombinant polypeptide comprising a nucleic acid binding domain (NBD) and a heterologous functional domain, the NBD comprising at least three repeat units (RUs) ordered from N-terminus to C-terminus of the NBD to specifically bind to a target nucleic acid, each of the RUs of the NBD comprising the consensus sequence: 1xxxx11x12xx33xxx1xxxxxxxxxx14xxx, where 1=A, F, I, L, M, T, V, or Y; 2=x or xx; 3=A, G, N, or S; 4=x, xx, or xxx; and x=any amino acid, and where each of the RUs independently comprises a 33-36 amino acid long sequence that is at least 70% identical to the amino acid sequence set forth in one of SEQ ID NOs: 2-9, 23-35, 85-89, and 131-137, where SEQ ID NOs: 2-9, 33, and 89 provide amino acid sequences of RUs identified in a L. quateirensis bacterium protein (SEQ ID NO:1), where SEQ ID NOs: 23-32, 34-35, and 133 provide amino acid sequences of RUs identified in a L. maceachernii bacterium protein (SEQ ID NO: 143), where SEQ ID NOs: 25, 131-132, and 138 provide amino acid sequences of RUs identified in a protein (SEQ ID NO: 139) from a bacterium of the order Legionellales, and where SEQ ID NOs: 85-88, 134-137, and 151 provide amino acid sequences of RUs identified in a protein (SEQ ID NO: 147) from a bacterium of the genus Francisella. In certain aspects, the NBD further comprises a half-repeat unit.

In certain aspects, the present disclosure provides a recombinant polypeptide comprising a nucleic acid binding domain (NBD) and a heterologous functional domain, the NBD comprising at least three repeat units (RUs) ordered from N-terminus to C-terminus of the NBD to specifically bind to a target nucleic acid, each of the RUs of the NBD comprising the consensus sequence: (F/L/Y)(D/G/N/S)(A/H/R/S/T/V)(D/E/K/Q)(E/H/Q)(I/L/V)(I/L/V)(C/H/K/R/S)(I/M/V)(A/V)(A/G/S)(H/N/R)(A/D/G/I/K/N/S/V)(G)(G)(A/G/S)(H/K/L/N/R)(N)(I/L)(A/D/E/I/K/V)(A/L/V)(I/M/V)(K/L/Q/T)(A/D/E/K/L/Q/S)(A/C/F/N/V/Y)(F/H/L/Q/Y)(A/D/H/P/ Q)(A/D/I/K/R/T/V)(F/L)(K/M/Q/R/S)(D/E/N/S)(F/L/M)(D/E/G/H/K/N) (SEQ ID NO: 154), where the consensus sequence is based upon the amino acid sequences of RUs identified in proteins from a bacterium of the order Legionellales, a L. quateirensis bacterium, and a L. maceachernii bacterium.

In certain aspects, the present disclosure provides a recombinant polypeptide comprising a nucleic acid binding domain (NBD) and a heterologous functional domain, the NBD comprising at least three repeat units (RUs) ordered from N-terminus to C-terminus of the NBD to specifically bind to a target nucleic acid, each of the RUs of the NBD comprising the consensus sequence:

(SEQ ID NO: 156)
YK(P/S)EDIIRLASH(D/G)GGSVNLEAVLRL(H/N)(P/S)QL(I/T)
(G/R)LG,

where the consensus sequence is based upon the amino acid sequences of RUs identified in a protein from a Francisella species bacterium.

In certain aspects, the present disclosure provides a recombinant polypeptide comprising a nucleic acid binding domain (NBD) and a heterologous functional domain, the NBD comprising at least three repeat units (RUs) ordered from N-terminus to C-terminus of the NBD to specifically bind to a target nucleic acid, each of the RUs of the NBD comprising the consensus sequence: (F/L)(N/S/G)(S/V/A/T/H)(E/Q/K)(Q/E)(I/L)(I/V)(R/S/K)(M/I)(V/A)(S/A)X₁₂X₁₃GG (G/A/S)(L/K/N)NL(K/I)AV(T/K/L)(A/D/K/S)(N/Y/C)(H/Y)(D/K)(D/A/V)L(Q/K/R)(N/D/E)(M/R)(G/K/E) (SEQ ID NO: 158), where X₁₂X₁₃=HK, HD, HA, HN, HG, NN, NG, RN, HI, HV, RT, HD, SN, HS, GS, or LN and where the consensus sequence is based upon the amino acid sequences of RUs identified in a protein from a L. quateirensis bacterium.

In certain aspects, the last RU in the NBD may be a half-repeat that is 15-20 amino acids long and comprises the consensus sequence: 1xxxx11x12xx33x, where 1=A, F, I, L, M, T, V, or Y; 2=x or xx; 3=A, G, N, or S; 4=x, xx, or xxx; and x=any amino acid.

In some aspects, the target nucleic acid is at least three nucleotides in length. In further aspects, the target nucleic acid sequence is DNA or RNA that is at least three nucleotides in length.

In certain aspects, the 12^{th} and 13^{th} amino acid residues in a repeat unit are designated as base-contacting residues (BCR) that determine the base (A, G, T, or C) to which the repeat unit binds. In certain aspects, the BCR in a repeat unit as provided herein may be replaced with BCR as disclosed herein or by a RVD thereby changing the base to which the repeat unit binds. In certain aspects, the BCR in a repeat unit as provided herein may be replaced with BCR identified in a repeat from a Legionella protein (e.g., SEQ ID NO: 1 or 143). In certain aspects, the BCR in a repeat unit as provided herein may be replaced with BCR identified in a repeat from a Legionellales protein (e.g., SEQ ID NO:139). In certain aspects, the BCR in a repeat unit as provided herein may be replaced with BCR identified in a repeat from a Francisella protein (e.g., SEQ ID NO:147). In certain aspects, the BCR in a repeat unit as provided herein may be replaced with BCR listed in Table 1 herein.

In some aspects, a naturally occurring or non-naturally occurring linker is positioned between the NBD and the functional domain. In some aspects, the functional domain comprises an enzyme, a transcriptional activation domain, a transcriptional repression domain, a biotinylation reagent, a DNA nucleotide modifier, or a fluorophore. In further aspects, the enzyme is a nuclease, a DNA modifying protein, or a chromatin modifying protein.

In further aspects, the nuclease is a cleavage domain or a half-cleavage domain. In still some aspects, the cleavage domain or half-cleavage domain comprises a type IIS restriction enzyme. In further aspects, the type IIS restriction enzyme comprises FokI or Bfil. In some aspects, FokI has at least 70%, at least 75%, at least 80%, at least 85%, at least 90%, at least 92%, at least 95%, at least 97%, or at least 99% sequence identity to SEQ ID NO: 11. In some aspects, FokI has a sequence of SEQ ID NO: 11.

In some aspects, the chromatin modifying protein is lysine-specific histone demethylase 1 (LSD1). In some aspects, the transcriptional activation domain comprises VP16, VP64, p65, p300 catalytic domain, TET1 catalytic domain, TDG, Ldb1 self-associated domain, SAM activator (VP64, p65, HSF1), VPR (VP64, p65, Rta). In some aspects, the transcriptional repressor domain comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2. In some aspects, the DNA nucleotide modifier is adenosine deaminase.

In some aspects, the functional domain enables genome editing, gene regulation, or imaging at the genomic locus comprising the target nucleic acid bound by the modular nucleic acid binding domain comprising the RUs as described herein. In some aspects, each of the repeat units has at least 70%, at least 75%, at least 80%, at least 85%, at least 90%, at least 92%, at least 94%, at least 95%, at least 96%, at least 98%, or at least 99% sequence identity to any one of SEQ ID NOs: 2-10, 23-35, 85-89, 131-137, and. In further aspects, the repeat unit has the amino acid sequence of any one of SEQ ID NOs: 2-10, 23-35, 85-89, 131-138, and 151-152.

In some aspects, the RU is derived from a wild-type protein from an animal pathogen. In some aspects, the RU comprises a modification of a wild-type protein. In some aspects, the modification enhances specific recognition of a target nucleotide, base pair, or both. In some aspects, the modification comprises 1 to 29 modifications. In further aspects, the animal pathogen protein has at least 70%, at least 75%, at least 80%, at least 85%, at least 90%, at least 92%, at least 95%, at least 97%, or at least 99% sequence identity to SEQ ID NO: 1. In some aspects, the animal pathogen protein is SEQ ID NO: 1.

In further aspects, the NBD includes 3-40 RUs, e.g., 3-35, 3-30, 4-35, 4-30, 5-35, 5-30, 6-30, 7-30, 8-30, 9-30, 10-30, 10-28, or 10-25 RUs. In certain aspects, the NBD binds to a target nucleic acid that is at least 3 nucleotides long, e.g., 3-35, 3-30, 4-35, 4-30, 5-35, 5-30, 6-30, 7-30, 8-30, 9-30, 10-30, 10-28, or 10-25 nucleotides long.

In further aspects, the target nucleic acid is within a PDCD1 gene, a CTLA4 gene, a LAG3 gene, a TET2 gene, a BTLA gene, a HAVCR2 gene, a CCR5 gene, a CXCR4 gene, a TRA gene, a TRB gene, a B2M gene, an albumin gene, a HBB gene, a HBA1 gene, a TTR gene, a NR3C1 gene, a CD52 gene, an erythroid specific enhancer of the BCL11A gene, a CBLB gene, a TGFBR1 gene, a SERPINA1 gene, a HBV genomic DNA in infected cells, a CEP290 gene, a DMD gene, a CFTR gene, an IL2RG gene, or a combination thereof. In other aspects, a chimeric antigen receptor (CAR), alpha-L iduronidase (IDUA), iduronate-2-sulfatase (IDS), or Factor 9 (F9), is inserted upon cleavage of a region of the target nucleic acid sequence.

In various aspects, the present disclosure provides a method of genome editing in a subject, wherein the method comprises: administering a non-naturally occurring modular nucleic acid binding domain comprising a functional domain, wherein the functional domain comprises a cleavage domain or a cleavage half domain; and inducing a double stranded break, wherein the modular nucleic acid binding domain comprises a modular nucleic acid binding domain derived from an animal pathogen protein (MAP-NBD), wherein the MAP-NBD comprises a plurality of repeat units and wherein the plurality of repeat units recognizes a target nucleic acid.

In some aspect, the method further comprises a second MAP-NBD wherein the second MAP-NBD comprises a second plurality of repeat units that recognizes a second target nucleic acid. In some aspects, the MAP-NBD, the second MAP-NBD, or both further comprise a functional domain, e.g., a cleavage domain or a cleavage half domain. In further aspects, the cleavage domain or the cleavage half domain comprises FokI or Bfil. In some aspects, the cleavage domain comprises a meganuclease.

In further aspects, FokI has at least 70%, at least 75%, at least 80%, at least 85%, at least 90%, at least 92%, at least 95%, at least 97%, or at least 99% sequence identity to SEQ ID NO: 11. In still further aspects, FokI has a sequence of SEQ ID NO: 11. In further aspects, the target nucleic acid sequence is within a PDCD1 gene, a CTLA4 gene, a LAG3 gene, a TET2 gene, a BTLA gene, a HAVCR2 gene, a CCR5 gene, a CXCR4 gene, a TRA gene, a TRB gene, a B2M gene, an albumin gene, a HBB gene, a HBA1 gene, a TTR gene, a NR3C1 gene, a CD52 gene, an erythroid specific enhancer of the BCL11A gene, a CBLB gene, a TGFBR1 gene, a SERPINA1 gene, a HBV genomic DNA in infected cells, a CEP290 gene, a DMD gene, a CFTR gene, an IL2RG gene, or a combination thereof. In other aspects, a chimeric antigen receptor (CAR), alpha-L iduronidase (IDUA), iduronate-2-sulfatase (IDS), or Factor 9 (F9), is inserted upon cleavage of a region of the target nucleic acid sequence.

In various aspects, the present disclosure provides a method of gene regulation in a subject, wherein the method comprises: administering a non-naturally occurring modular nucleic acid binding domain; and regulating expression of a gene, wherein the modular nucleic acid binding domain comprises a modular DNA binding domain derived from an animal pathogen protein (MAP-NBD) and wherein the MAP-NBD comprises a plurality of repeat units and wherein a repeat unit of the plurality of repeat units recognizes a target nucleic acid.

In further aspects, the MAP-NBD further comprises a functional domain. In some aspects, the functional domain comprises a transcriptional activation domain or a transcriptional repression domain. In some aspects, the activation domain comprises VP16, VP64, p65, p300 catalytic domain, TET1 catalytic domain, TDG, Ldb1 self-associated domain, SAM activator (VP64, p65, HSF1), VPR (VP64, p65, Rta). In some aspects, the repressor domain comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

In some aspects, the target nucleic acid sequence is within a PDCD1 gene, a CTLA4 gene, a LAG3 gene, a TET2 gene, a BTLA gene, a HAVCR2 gene, a CCR5 gene, a CXCR4 gene, a TRA gene, a TRB gene, a B2M gene, an albumin gene, a HBB gene, a HBA1 gene, a TTR gene, a NR3C1 gene, a CD52 gene, an erythroid specific enhancer of the BCL11A gene, a CBLB gene, a TGFBR1 gene, a SERPINA1 gene, a HBV genomic DNA in infected cells, a CEP290 gene, a DMD gene, a CFTR gene, or a combination thereof.

In various aspects, the present disclosure provides a method of imaging a genomic locus in vivo in a subject, wherein the method comprises: administering to the subject a non-naturally occurring modular nucleic acid binding domain conjugated to an imaging agent; and imaging the subject, wherein the modular nucleic acid binding domain comprises a modular DNA binding domain derived from an animal pathogen protein (MAP-NBD) and wherein the MAP-NBD comprises a plurality of repeat units that recognizes a target nucleic acid. In some aspects, the imaging agent is a fluorescent moiety. In some aspects, the fluorescent moiety is GFP or mCHERRY. In some aspects, the target nucleic acid is a single nucleotide, a single base pair, or both. In some aspects, the target nucleic acid is DNA or RNA. In some aspects, the MAP-NBD recognizes a target nucleic acid sequence. In some aspects, the MAP-NBD binds the target nucleic acid sequence. In some aspects, the target nucleic acid sequence is DNA or RNA. In some aspects, the composition further comprises a linker between the MAP-NBD and the functional domain. In some aspects, the animal pathogen protein is derived from a bacterium. In further aspects, the bacterium is selected from the genus of Legionella. In some aspects, the bacterium is L. quateirensis. In some aspects, the repeat unit comprises a consensus sequence of

(SEQ ID NO: 19)
1xxx211x1xxx33x2x1xxxxxxxxx1.

In some aspects, the repeat unit has at least 70%, at least 75%, at least 80%, at least 85%, at least 90%, at least 92%, at least 95%, at least 97%, or at least 99% sequence identity to any one of SEQ ID NOs: 2-10, 23-35, 85-89, and 131-137. In some aspects, the repeat unit has the amino acid sequence of any one of SEQ ID NOs: 2-9, 23-35, 85-89, 131-138 or 151.

In some aspects, the repeat unit is derived from a wild-type protein. In some aspects, the repeat unit comprises a modification of a wild-type protein. In some aspects, the modification enhances specific recognition of a target nucleotide. In some aspects, the modification comprises 1 to 29 modifications. In some aspects, the animal pathogen protein has at least 70%, at least 75%, at least 80%, at least 85%, at least 90%, at least 92%, at least 95%, at least 97%, or at least 99% sequence identity to SEQ ID NO: 1. In further aspects, the animal pathogen protein is SEQ ID NO: 1. In some aspects, the genomic locus is in a cell. In some aspects, the cell is in a plurality of cells ex vivo, in a human, or in a non-human animal.

A method for producing a polypeptide that specifically binds to a target DNA sequence is disclosed. The method included synthesizing a polypeptide comprising a DNA binding domain (DBD) that specifically binds to the target sequence, where the DBD comprises repeat units that are selected based on the DNA base bound by the repeat unit and combined in the appropriate order to match the target DNA sequence, where when the target sequence includes an adenine (A), the repeat unit comprises a 33-35 amino acid long sequence that is at least 70% identical to FSSQQIIRMVSHAGGANNLKAVTANHDDLQNMG (SEQ ID NO:2), or LGHKELIKIAARNGGGNNLIAVLSCYAKLKEMG (SEQ ID NO:89), or comprises the sequence of SEQ ID NO:2 or SEQ ID NO:89 comprising conservative amino acid substitutions; when the target sequence includes a thymine (T), the repeat unit comprises a 33-35 amino acid long sequence that is at least 70% identical to: FNAEQIVRMVSHGGGSKNLKAVTDNHDDLKNMG (SEQ ID NO: 4); FNAEQIVSMVSNGGGSLNLKAVKKYHDALKDRG (SEQ ID NO:6); or FNVEQIVSIVSHGGGSLNLKAVKKYHDVLKDRE (SEQ ID NO:8), or comprises the sequence of SEQ ID NOs: 4, 6, or 8 comprising conservative amino acid substitutions; when the target sequence includes a cytosine (C), the repeat unit comprises a 33-35 amino acid long sequence that is at least 70% identical to: FNTEQIVRMVSHDGGSLNLKAVKKYHDALRERK (SEQ ID NO:7); or FNAEQIVRMVSHDGGSLNLKAVTDNHDDLKNMG (SEQ ID NO:9), or comprises the sequence of SEQ ID NOs: 7 or 9 comprising conservative amino acid substitutions; when the target sequence includes a guanine (G), the repeat unit comprises a 33-35 amino acid long sequence that is at least 70% identical to: FNVEQIVRMVSHNGGSKNLKAVTDNHDDLKNMG (SEQ ID NO:3); FNAEQIVSMVSNNGGSKNLKAVTDNHDDLKNMG (SEQ ID NO:5); FNAEQIVRMVSHKGGSKNLALVKEYFPVFSSFH (SEQ ID NO:33); or LGHKELIKIAARNGGGNNLIAVLSCYAKLKEMG (SEQ ID NO:89), or comprises the sequence of SEQ ID NOs: 3, 5, 33, or 89 comprising conservative amino acid substitutions.

An additional method for producing a polypeptide that specifically binds to a target DNA sequence is disclosed. The method includes synthesizing a polypeptide comprising a DNA binding domain (DBD) that specifically binds to the target sequence, wherein the DBD comprises repeat units that are selected based on the DNA base bound by the repeat unit and combined in the appropriate order to match the target DNA sequence, where: when the target sequence includes an adenine (A), the repeat unit comprises a 33-35 amino acid long sequence that is at least 70% identical to FSAKHIVRIAAHIGGSLNIKAVQQAQQALKELG (SEQ ID NO:32), FSAEQIVSIAAHVGGSHNIEAVQKAHQALKELD (SEQ ID NO:35), FSAEQIVRIAAHIGGSHNLKAVLQAQQALKELD (SEQ ID NO:31), or FSAEQIVRIAAHIGGSRNIEATIKHYAMLTQPP (SEQ ID NO:133), or comprises the sequence of SEQ ID NOs: 32, 36, 31, or 133 comprising conservative amino acid substitutions; when the target sequence includes a thymine (T), the repeat unit comprises a 33-35 amino acid long sequence that is at least 70% identical to: YSSEQIVRVAAHGGGSLNIKAVLQAHQALKELD (SEQ ID NO:28), FSAEQIVHIAAHGGGSLNIKAILQAHQTLKELN (SEQ ID NO:29), FSTEQIVCIAGHGGGSLNIKAVLLAQQALKDLG (SEQ ID NO:27), FSAEQIVSIAAHVGGSHNIEAVQKAHQALKELD (SEQ ID NO:35), or FSAEQIVRIAAHIGGSRNIEATIKHYAMLTQPP (SEQ ID NO:133), or comprises the sequence of SEQ ID NOs: 28, 29, 27, 35, or 133 comprising conservative amino acid substitutions; when the target sequence includes a cytosine (C), the repeat unit comprises a 33-35 amino acid long sequence that is at least 70% identical to: FSAEQIVSIVAHDGGSRNIEAVQQAQHILKELG (SEQ ID NO: 24), FSAEQIVRIAAHDGGSLNIDAVQQAQQALKELG (SEQ ID NO:26), or FSAEQIVRIAAHIGGSHNLKAVLQAQQALKELD (SEQ ID NO:31), or comprises the sequence of SEQ ID NOs: 24, 26, or 31 comprising conservative amino acid substitutions; when the target sequence includes a guanine (G), the repeat unit comprises a 33-35 amino acid long sequence that is at least 70% identical to: FSAEQIVRIAAHIGGSRNIEAIQQAHHALKELG (SEQ ID NO:30), FSADQIVRIAAHKGGSHNIVAVQQAQQALKELD (SEQ ID NO:34), FSAEQIVSIAAHVGGSHNIEAVQKAHQALKELD (SEQ ID NO:35), or FSAEQIVRIAAHIGGSRNIEATIKHYAMLTQPP (SEQ ID NO:133), or comprises the sequence of SEQ ID NOs: 30, 34, 35, or 133 comprising conservative amino acid substitutions.

In certain aspects of the method, the DBD may include any of the repeat units disclosed herein, for example, a DBD may include repeat units derived from different Legionella proteins as provided herein. The target sequence may be in a promoter region or the open reading frame of a gene of interest. The DBD may be conjugated to a functional domain, as provided herein.

### BRIEF DESCRIPTION OF THE DRAWINGS

FIG. 1 is a schematic of an embodiment of a polypeptide disclosed herein, comprising a nucleic acid binding domain (NBD) made of a plurality of repeat units. Each repeat unit has the consensus sequence 1xxxx11x12xx33xxx1xxxxxxxxxx14xxx. The last repeat unit is a half-repeat having a length of 15-20 amino acids. The NBD is flanked by an N-terminal domain and a C-terminal domain.

FIG. 2 illustrates a protein from L. quateirensis from which nucleic acid binding repeat domains can be derived. SEQ ID NO: 1 provides the amino acid sequence of the full protein which contains repeats units (RU) having sequences set forth in SEQ ID NOs: 89 and 2-SEQ ID NO: 10. The amino acid sequence set forth in SEQ ID NO: 89 extends from the residue at position 144 to the residue at position 176 of SEQ ID NO:1. The amino acid sequence set forth in SEQ ID NO: 2 extends from the residue at position 177 to the residue at position 209 of SEQ ID NO:1. The amino acid sequence set forth in SEQ ID NO: 3 extends from the residue at position 210 to the residue at position 242 of SEQ ID NO:1. The amino acid sequence set forth in SEQ ID NO: 4 extends from the residue at position 243 to the residue at position 275 of SEQ ID NO:1. The amino acid sequence set forth in SEQ ID NO: 5 extends from the residue at position 276 to the residue at position 308 of SEQ ID NO: 1. The amino acid sequence set forth in SEQ ID NO: 6 extends from the residue at position 309 to the residue at position 341 of SEQ ID NO:1. The amino acid sequence set forth in SEQ ID NO: 7 extends from the residue at position 342 to the residue at position 374 of SEQ ID NO:1. The amino acid sequence set forth in SEQ ID NO: 8 extends from the residue at position 375 to the residue at position 407 of SEQ ID NO:1. The amino acid sequence set forth in SEQ ID NO: 9 extends from the residue at position 408 to the residue at position 440 of SEQ ID NO:1. The amino acid sequence set forth in SEQ ID NO: 10 extends from the residue at position 441 to the residue at position 459 of SEQ ID NO:1. The RU having the amino acid sequence of SEQ ID NO:10 is a half-repeat unit.

FIG. 3A shows consensus sequences that can be present in an animal pathogen-derived protein. Each consensus sequence includes a repeat motif, followed by a spacer of variable length, and a second repeat motif.

FIG. 3B shows a consensus sequence which can be present in a repeat domain (e.g., a MAP-NBD) of the present disclosure.

FIG. 4A illustrates the bases to which the repeats (SEQ ID NOs: 89 and 2-10 ordered from N-terminus to C-terminus) in a polypeptide having the amino acid sequence set forth in SEQ ID NO: 1 can bind. The polypeptide having the amino acid sequence set forth in SEQ ID NO:1 was fused to a His-tag. The larger the size the of the base at a particular position, the higher the relative frequency at which the base is present at that position in a nucleic acid bound by the tested polypeptide.

FIG. 4B illustrates the bases to which the repeats (from N-terminus to C-terminus: SEQ ID NOs: 89 and 2-10) in a polypeptide having the amino acid sequence set forth in SEQ ID NO:1 can bind. The polypeptide having the amino acid sequence set forth in SEQ ID NO: 1 was fused to a HA-tag. The larger the size the of the base at a particular position, the higher the relative frequency at which the base is present at that position in a nucleic acid bound by the tested polypeptide. Both the His-tagged polypeptide and the HA-tagged polypeptide showed the same binding specificity.

FIG. 5 summarizes the BCR present in repeat units having the amino acid sequence set forth in SEQ ID NOs: 89 and 2-10, respectively and the corresponding base to which the repeat unit bind.

FIG. 6 shows that while most of the BCRs in the repeats units derived from L. quateirensis show specificity for the same base as RVDs in the repeats from Xanthomonas as reported in Miller et al. Nature Methods, 12 (5), 465-471, 2015, the BCR “HA” in a L. quateirensis derived repeat having the sequence of SEQ ID NO:2 specify binding to “A” rather than binding to “T” as observed for the repeat containing the RVD HA from Xanthomonas.

FIG. 7 provides the amino acid sequence (SEQ ID NO:139) of a protein from a Legionellales bacterium. The protein includes four repeat units.

FIG. 8A depicts the bases to which the BCR in repeats (having the amino acid sequence set forth in SEQ ID NOs: 89, 2-9, and 33) ordered from N-terminus to C-terminus in a polypeptide having the amino acid sequence set forth in SEQ ID NO:1 can bind. The larger the size the of the base at a particular position, the higher the relative frequency at which the base is present at that position in a nucleic acid bound by the tested polypeptide. The N-terminus (SEQ ID NO:13) of the protein and the C-terminus (SEQ ID NO:159) of the protein did not show binding to any base.

FIG. 8B depicts the bases to which the BCR in repeats (having the amino acid sequence set forth in SEQ ID NOs: 28, 26, 32, 34, 30, 31, 23, 29, 24, 27, 35, and 133) ordered from N-terminus to C-terminus in a polypeptide having the amino acid sequence set forth in SEQ ID NO:143 can bind. The larger the size the of the base at a particular position, the higher the relative frequency at which the base is present at that position in a nucleic acid bound by the tested polypeptide. The N-terminus (SEQ ID NO:144) of the protein mediated binding to either G or the sequence G-G. The C-terminus (SEQ ID NO:145) of the protein potentially mediated binding to the sequence T or T-T.

FIG. 9 illustrates that the protein the BCR in repeats (having the amino acid sequence set forth in SEQ ID NOs: 28, 26, 32, 34, 30, 31, 23, 29, 24, 27, 35, and 133) ordered from N-terminus to C-terminus in a polypeptide having the amino acid sequence set forth in SEQ ID NO:143 binds to the same DNA sequence in vivo as discovered by SELEX-seq.

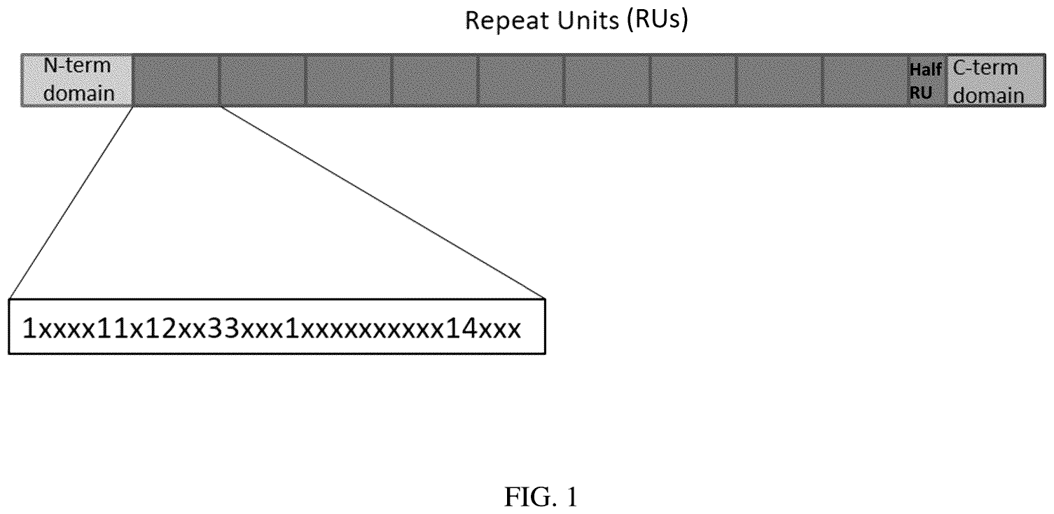

### DETAILED DESCRIPTION

Provided herein are polypeptides, compositions, and methods of use thereof for genetic and epigenomic engineering, including, genome editing and gene regulation. These polypeptides and compositions include nucleic acid binding domains that bind to a target nucleic acid of interest. The nucleic acid binding domains include repeat units derived from repeat units identified in proteins from animal pathogens such as bacterium of the order Legionellales and the species Legionella and Francisella.

### Definitions

As used herein, the term “derived” in the context of a polypeptide refers to a polypeptide that has a sequence that is based on that of a protein from a particular source (e.g., an animal pathogen such as Legionella). A polypeptide derived from a protein from a particular source may be a variant of the protein from the particular source (e.g., an animal pathogen such as Legionella). For example, a polypeptide derived from a protein from a particular source may have a sequence that is modified with respect to the protein's sequence from which it is derived. A polypeptide derived from a protein from a particular source shares at least 30% sequence identity with, at least 40% sequence identity with, at least 50% sequence identity with, at least 60% sequence identity with, at least 70% sequence identity with, at least 80% sequence identity with, or at least 90% sequence identity with the protein from which it is derived.

The term “modular” as used herein in the context of a nucleic acid binding domain, e.g., a modular animal pathogen derived nucleic acid binding domain (MAP-NBD) indicates that the plurality of repeat units present in the NBD can be rearranged and/or replaced with other repeat units and can be arranged in an order such that the NBD binds to the target nucleic acid. For example, any repeat unit in a modular nucleic acid binding domain can be switched with a different repeat unit. In some embodiments, modularity of the nucleic acid binding domains disclosed herein allows for switching the target nucleic acid base for a particular repeat unit by simply switching it out for another repeat unit. In some embodiments, modularity of the nucleic acid binding domains disclosed herein allows for swapping out a particular repeat unit for another repeat unit to increase the affinity of the repeat unit for a particular target nucleic acid. Overall, the modular nature of the nucleic acid binding domains disclosed herein enables the development of genome editing complexes that can precisely target any nucleic acid sequence of interest.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence, fusion proteins with heterologous and homologous leader sequences, with or without N-terminus methionine residues; immunologically tagged proteins; and the like. In specific embodiments, the terms refer to a polymeric form of amino acids of any length which include genetically coded amino acids. In particular embodiments, the terms refer to a polymeric form of amino acids of any length which include genetically coded amino acids fused to a heterologous amino acid sequence.

The term “heterologous” refers to two components that are defined by structures derived from different sources. For example, in the context of a polypeptide, a “heterologous” polypeptide may include operably linked amino acid sequences that are derived from different polypeptides (e.g., a NBD and a functional domain derived from different sources). Similarly, in the context of a polynucleotide encoding a chimeric polypeptide, a “heterologous” polynucleotide may include operably linked nucleic acid sequences that can be derived from different genes. Other exemplary “heterologous” nucleic acids include expression constructs in which a nucleic acid comprising a coding sequence is operably linked to a regulatory element (e.g., a promoter) that is from a genetic origin different from that of the coding sequence (e.g., to provide for expression in a host cell of interest, which may be of different genetic origin than the promoter, the coding sequence or both). In the context of recombinant cells, “heterologous” can refer to the presence of a nucleic acid (or gene product, such as a polypeptide) that is of a different genetic origin than the host cell in which it is present.

The term “operably linked” refers to linkage between molecules to provide a desired function. For example, “operably linked” in the context of nucleic acids refers to a functional linkage between nucleic acid sequences. By way of example, a nucleic acid expression control sequence (such as a promoter, signal sequence, or array of transcription factor binding sites) may be operably linked to a second polynucleotide, wherein the expression control sequence affects transcription and/or translation of the second polynucleotide. In the context of a polypeptide, “operably linked” refers to a functional linkage between amino acid sequences (e.g., different domains) to provide for a described activity of the polypeptide.

As used herein, the term “cleavage” refers to the breakage of the covalent backbone of a nucleic acid, e.g., a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, the polypeptides provided herein are used for targeted double-stranded DNA cleavage.

A “cleavage half-domain” is a polypeptide sequence which, in conjunction with a second polypeptide (either identical or different) forms a complex having cleavage activity (preferably double-strand cleavage activity).

A “target nucleic acid,” “target sequence,” or “target site” is a nucleic acid sequence that defines a portion of a nucleic acid to which a binding molecule, such as, the NBD disclosed herein will bind. The target nucleic acid may be present in an isolated form or inside a cell. A target nucleic acid may be present in a region of interest. A “region of interest” may be any region of cellular chromatin, such as, for example, a gene or a non-coding sequence within or adjacent to a gene, in which it is desirable to bind an exogenous molecule. Binding can be for the purposes of targeted DNA cleavage and/or targeted recombination, targeted activated or repression. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, promoter sequences, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

An “exogenous” molecule is a molecule that is not normally present in a cell but can be introduced into a cell by one or more genetic, biochemical or other methods. An exogenous molecule can comprise, for example, a functioning version of a malfunctioning endogenous molecule, e.g. a gene or a gene segment lacking a mutation present in the endogenous gene. An exogenous nucleic acid can be present in an infecting viral genome, a plasmid or episome introduced into a cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control region.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA, shRNA, RNAi, miRNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristylation, and glycosylation.

“Modulation” of gene expression refers to a change in the activity of a gene. Modulation of expression can include, but is not limited to, gene activation and gene repression. Genome editing (e.g., cleavage, alteration, inactivation, donor integration, random mutation) can be used to modulate expression. Gene inactivation refers to any reduction in gene expression as compared to a cell that does not include a polypeptide or has not been modified by a polypeptide as described herein. Thus, gene inactivation may be partial or complete.

The terms “patient” or “subject” are used interchangeably to refer to a human or a non-human animal (e.g., a mammal).

The terms “treat”, “treating”, treatment” and the like refer to a course of action (such as administering a polypeptide comprising a NBD fused to a heterologous functional domain or a nucleic acid encoding the polypeptide) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject.

The terms “prevent”, “preventing”, “prevention” and the like refer to a course of action (such as administering a polypeptide comprising a NBD fused to a heterologous functional domain or a nucleic acid encoding the polypeptide) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

The phrase “therapeutically effective amount” refers to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to a patient. The therapeutically effective amount can be ascertained by measuring relevant physiological effects.

### Animal Pathogen Derived Nucleic Acid Binding Domains

The present disclosure provides a modular nucleic acid binding domain (NBD) and methods of using modular NBDs. A modular NBD can be engineered to target and bind to a specific nucleic acid sequence. The nucleic acid sequence can be DNA or RNA. In some embodiments, the modular NBD can comprise a plurality of repeat domains, wherein each repeat domain recognizes and binds to a single nucleotide or base pair. Each repeat domain in the plurality of repeat domains can be specifically selected to target and bind to a specific nucleic acid sequence, thus contributing to the modular nature of the NBD. A non-naturally occurring modular nucleic acid binding domain derived from an animal pathogen protein (MAP-NBD) can comprise a plurality of repeat units, wherein a repeat unit of the plurality of repeat units recognizes a single target nucleotide, base pair, or both.

In some embodiments, the repeat domain can be derived from an animal pathogen and can be referred to as a non-naturally occurring modular nucleic acid binding domain derived from an animal pathogen protein (MAP-NBD), or “modular animal pathogen-nucleic acid binding domain” (MAP-NBD). For example, in some cases, the animal pathogen can be from the Gram-negative bacterium genus, Legionella. In other cases, the animal pathogen can be a bacterium from the genus Burkholderia. In some cases, the animal pathogen can be a bacterium from the genus Paraburkholderia. In other cases, the animal pathogen can be a bacterium from the genus Francisella.

In certain aspects, the NBD such as a MAP-NBD comprises RUs derived from the animal pathogen, L. quateirensis. In certain aspects, the RUs are derived from RUs identified in a protein from L. quateirensis, where the protein has the amino acid sequence set forth in SEQ ID NO:1.

In the context of a repeat unit(s), the terms “repeat(s),” “repeat unit(s),” “repeat motif(s),” “repeat domain(s),” and “repeat sequence(s)” are used interchangeably.

In particular embodiments, the repeat domain can be derived from a Legionellales bacterium, a species of the genus of Legionella, such as L. quateirensis or L. maceachernii, the genus of Burkholderia, the genus of Paraburkholderia, or the genus of Francisella. In some embodiments, the repeat domain can comprise from 19 amino acid residues to 36 amino acid residues, such as, 19-35 amino acids, 20-36 amino acids, 30-36 amino acids, 31-36 amino acids, 32-36 amino acids, 33-35 amino acids, or 33-36 amino acids. In particular embodiments, the repeat domain can comprise 33 amino acid residues. In other embodiments, the repeat unit can comprise 35 amino acid residues. In some embodiments, the MAP-NBD is non-naturally occurring, and comprises a plurality of repeat units ordered from N-terminus to C-terminus of the MAP-NBD to recognize a target nucleic acid.

In some embodiments, a repeat domain can be derived from a L. quateirensis protein with the following sequence:

(SEQ ID NO: 1)
MPDLELNFAIPLHLFDDETVFTHDATNDNSQASSSYSSKSSPASANARKR
TSRKEMSGPPSKEPANTKSRRANSQNNKLSLADRLTKYNIDEEFYQTRSD
SLLSLNYTKKQIERLILYKGRTSAVQQLLCKHEELLNLISPDGLGHKELI
KIAARNGGGNNLIAVLSCYAKLKEMGFSSQQIIRMVSHAGGANNLKAVTA
NHDDLQNMGFNVEQIVRMVSHNGGSKNLKAVTDNHDDLKNMGFNAEQIVR
MVSHGGGSKNLKAVTDNHDDLKNMGFNAEQIVSMVSNNGGSKNLKAVTDN
HDDLKNMGFNAEQIVSMVSNGGGSLNLKAVKKYHDALKDRGENTEQIVRM
VSHDGGSLNLKAVKKYHDALRERKFNVEQIVSIVSHGGGSLNLKAVKKYH
DVLKDREFNAEQIVRMVSHDGGSLNLKAVTDNHDDLKNMGFNAEQIVRMV
SHKGGSKNLALVKEYFPVFSSFHFTADQIVALICQSKQCFRNLKKNHQQW
KNKGLSAEQIVDLILQETPPKPNFNNTSSSTPSPSAPSFFQGPSTPIPTP
VLDNSPAPIFSNPVCFFSSRSENNTEQYLQDSTLDLDSQLGDPTKNFNVN
NFWSLFPFDDVGYHPHSNDVGYHLHSDEESPFFDF.

As demonstrated in Example 8 herein, repeat units have been identified in this L. quateirensis protein. The repeats found in TALE proteins from Xanthomonas have highly conserved amino acid sequence other than at positions 12^{th} and 13^{th}. The amino acids present at positions 12^{th} and 13^{th} in repeats in Xanthomonas TALE proteins are referred to as repeat variable di-residues (RVDs) since in most instances only amino acids present at these two positions vary between repeats in Xanthomonas TALE proteins. In contrast, the repeat units identified in this L. quateirensis protein additionally have sequence variation outside of the 12^{th} and 13^{th} amino acid positions. However, as demonstrated in Example 8, the amino acids present at 12^{th} and 13^{th} positions in the RUs disclosed herein mediate binding to a particular base. These residues are herein referred to as base-contacting residues (BCR).

In some embodiments, a repeat from a L. quateirensis protein can comprise BCR that have the same sequence of di-residues as that of a RVD in a repeat from a TALE protein from Xanthomonas. Such BCR are referred to as canonical BCR. In some embodiments, canonical BCR can comprise the residues NN, NG, or HD. In some embodiments, a repeat from a L. quateirensis protein can comprise BCR that have a different sequence of residues than a RVD in a repeat from a TALE protein from Xanthomonas. Such BCR are referred to as non-canonical BCR. In some embodiments, non-canonical BCR can comprise the residues RN, HA, HN, HG, or HK.

In some embodiments, a repeat of SEQ ID NO: 89 comprises the BCR RN and recognizes the base guanine (G). In some embodiments, a repeat of SEQ ID NO: 2 comprises the BCR HA and primarily recognizes the base adenine (A). In some embodiments, a repeat of SEQ ID NO: 3 comprises the BCR HN and recognizes the base G. In some embodiments, a repeat of SEQ ID NO: 4 comprises the BCR HG and recognizes the base thymine (T). In some embodiments, a repeat of SEQ ID NO: 5 comprises the BCR NN and recognizes the base G. In some embodiments, a repeat of SEQ ID NO: 6 comprises the BCR NG and recognizes the base T. In some embodiments, a repeat of SEQ ID NO: 7 comprises the BCR HD and recognizes the base cytosine (C). In some embodiments, a repeat of SEQ ID NO: 8 comprises the BCR HG and recognizes the base T. In some embodiments, a repeat of SEQ ID NO: 9 comprises the BCR HD and recognizes the base C. In some embodiments, a half-repeat of SEQ ID NO: 10 comprises the BCR HK and recognizes the base G.

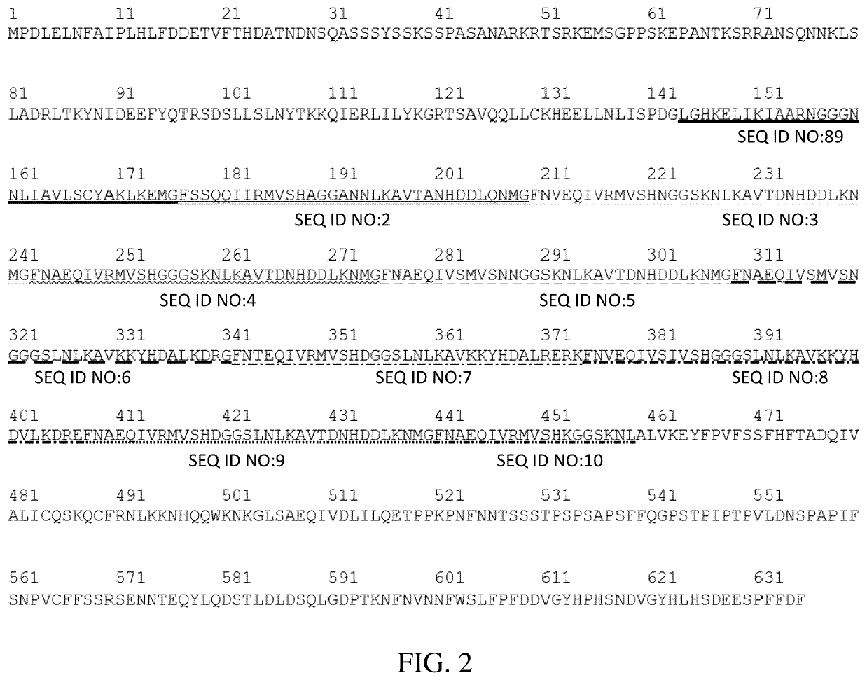

FIG. 2 illustrates a protein from L. quateirensis from which nucleic acid binding repeat domains are derived. SEQ ID NO: 1 indicates the full protein and contains repeats of SEQ ID NO: 2-SEQ ID NO: 10 and SEQ ID NO: 89.

TABLE 1 illustrates exemplary repeats from Legionalleles, L. quateirensis, L. maceachernii Burkholderia, Paraburkholderia, or Francisella that can make up a MAP-NBD of the present disclosure. The BCR (i.e., the amino acids present at position 12 and 13) of the particular repeat is also indicated. A MAP-NBD of the present disclosure can comprise at least one of the repeats disclosed in TABLE 1. A MAP-NBD of the present disclosure can comprise any combination of repeats disclosed in TABLE 1.

TABLE 1
Animal Pathogen Repeat Domains

| SEQ | | | |
|---|---|---|---|
| ID NO | Organism | Repeat Domain Sequence | BCR |
| 2 | L. quateirensis | FSSQQIIRMVSHAGGANNLKAVTANHDDLQNMG | HA |
| 3 | L. quateirensis | FNVEQIVRMVSHNGGSKNLKAVTDNHDDLKNMG | HN |
| 4 | L. quateirensis | FNAEQIVRMVSHGGGSKNLKAVIDNHDDLKNMG | HG |
| 5 | L. quateirensis | FNAEQIVSMVSNNGGSKNLKAVIDNHDDLKNMG | NN |
| 6 | L. quateirensis | FNAEQIVSMVSNGGGSLNLKAVKKYHDALKDRG | NG |
| 7 | L. quateirensis | FNTEQIVRMVSHDGGSLNLKAVKKYHDALRERK | HD |
| 8 | L. quateirensis | FNVEQIVSIVSHGGGSLNLKAVKKYHDVLKDRE | HG |
| 9 | L. quateirensis | FNAEQIVRMVSHDGGSLNLKAVTDNHDDLKNMG | HD |
| 10 | L. quateirensis | FNAEQIVRMVSHKGGSKNL | HK |
| (half- | | | |
| repeat) | | | |
| 23 | Legionella | FSAEQIVRIAAHDGGSRNIEAVQQAQHVLKELG | HD |
| | maceachernii | | |
| 24 | Legionella | FSAEQIVSIVAHDGGSRNIEAVQQAQHILKELG | HD |
| | maceachernii | | |
| 25 | Legionellales | LDRQQILRIASHDGGSKNIAAVQKFLPKLMNFG | HD |
| | bacterium | | |
| 26 | Legionella | FSAEQIVRIAAHDGGSLNIDAVQQAQQALKELG | HD |
| | maceachernii | | |
| 27 | Legionella | FSTEQIVCIAGHGGGSLNIKAVLLAQQALKDLG | HG |
| | maceachernii | | |
| 28 | Legionella | YSSEQIVRVAAHGGGSLNIKAVLQAHQALKELD | HG |
| | maceachernii | | |
| 29 | Legionella | FSAEQIVHIAAHGGGSLNIKAILQAHQTLKELN | HG |
| | maceachernii | | |
| 30 | Legionella | FSAEQIVRIAAHIGGSRNIEAIQQAHHALKELG | HI |
| | maceachernii | | |
| 31 | Legionella | FSAEQIVRIAAHIGGSHNLKAVLQAQQALKELD | HI |
| | maceachernii | | |
| 32 | Legionella | FSAKHIVRIAAHIGGSLNIKAVQQAQQALKELG | HI |
| | maceachernii | | |
| 33 | L. quateirensis | FNAEQIVRMVSHKGGSKNLALVKEYFPVFSSFH | HK |
| 34 | Legionella | FSADQIVRIAAHKGGSHNIVAVQQAQQALKELD | HK |
| | maceachernii | | |
| 35 | Legionella | FSAEQIVSIAAHVGGSHNIEAVQKAHQALKELD | HV |
| | maceachernii | | |
| 36 | Burkholderia | FSSGETVGATVGAGGTETVAQGGTASNTTVSSG | GA |
| 37 | Burkholderia | FSGGMATSTTVGSGGTQDVLAGGAAVGGTVGTG | GS |
| 38 | Burkholderia | FSAADIVKIAGKIGGAQALQAFITHRAALIQAG | KI |
| 39 | Burkholderia | FNPTDIVKIAGNDGGAQALQAVLELEPALRERG | ND |
| 40 | Burkholderia | FNPTDIVRMAGNDGGAQALQAVFELEPAFRERS | ND |
| 41 | Burkholderia | FNPTDIVRMAGNDGGAQALQAVLELEPAFRERG | ND |
| 42 | Burkholderia | FSQVDIVKIASNDGGAQALYSVLDVEPTFRERG | ND |
| 43 | Burkholderia | FSRADIVKIAGNDGGAQALYSVLDVEPPLRERG | ND |
| 44 | Burkholderia | FSRGDIVKIAGNDGGAQALYSVLDVEPPLRERG | ND |
| 45 | Burkholderia | FNRADIVRIAGNGGGAQALYSVRDAGPTLGKRG | NG |
| 46 | Burkholderia | FRQADIVKIASNGGSAQALNAVIKLGPTLRQRG | NG |
| 47 | Burkholderia | FRQADIVKMASNGGSAQALNAVIKLGPTLRQRG | NG |
| 48 | Burkholderia | FSRADIVKIAGNGGGAQALQAVLELEPTFRERG | NG |
| 49 | Burkholderia | FSRADIVRIAGNGGGAQALYSVLDVGPTLGKRG | NG |
| 50 | Burkholderia | FSRGDIVRIAGNGGGAQALQAVLELEPTLGERG | NG |
| 51 | Burkholderia | FSRADIVKIAGNGGGAQALQAVITHRAALTQAG | NG |
| 52 | Burkholderia | FSRGDTVKIAGNIGGAQALQAVLELEPTLRERG | NI |
| 53 | Burkholderia | FNPTDIVKIAGNIGGAQALQAVLELEPAFRERG | NI |
| 54 | Burkholderia | FSAADIVKIAGNIGGAQALQAIFTHRAALIQAG | NI |
| 55 | Burkholderia | FSAADIVKIAGNIGGAQALQAVITHRATLIQAG | NI |
| 56 | Burkholderia | FSATDIVKIASNIGGAQALQAVISRRAALIQAG | NI |
| 57 | Burkholderia | FSQPDIVKIAGNIGGAQALQAVLELEPAFRERG | NI |
| 58 | Burkholderia | FSRADIVKIAGNIGGAQALQAVLELESTFRERS | NI |
| 59 | Burkholderia | FSRADIVKIAGNIGGAQALQAVLELESTLRERS | NI |
| 60 | Burkholderia | FSRGDIVKMAGNIGGAQALQAGLELEPAFRERG | NI |
| 61 | Burkholderia | FSRGDIVKMAGNIGGAQALQAVLELEPAFHERS | NI |
| 62 | Burkholderia | FTLTDIVKMAGNIGGAQALKAVLEHGPTLRQRD | NI |
| 63 | Burkholderia | FTLTDIVKMAGNIGGAQALKVVLEHGPTLRQRD | NI |
| 64 | Burkholderia | FNPTDIVKIAGNNGGAQALQAVLELEPALRERG | NN |
| 65 | Burkholderia | FNPTDIVKIAGNNGGAQALQAVLELEPALRERS | NN |
| 66 | Burkholderia | FNPTDMVKIAGNNGGAQALQAVLELEPALRERG | NN |
| 67 | Burkholderia | FSAADIVKIASNNGGAQALQALIDHWSTLSGKT | NN |
| 68 | Burkholderia | FSAADIVKIASNNGGAQALQAVISRRAALIQAG | NN |
| 69 | Burkholderia | FSAADIVKIASNNGGAQALQAVITHRAALAQAG | NN |
| 70 | Burkholderia | FSAADIVKIASNNGGARALQALIDHWSTLSGKT | NN |
| 71 | Burkholderia | FTLTDIVEMAGNNGGAQALKAVLEHGSTLDERG | NN |
| 72 | Burkholderia | FTLTDIVKMAGNNGGAQALKAVLEHGPTLDERG | NN |
| 73 | Burkholderia | FTLTDIVKMAGNNGGAQALKVVLEHGPTLRQRG | NN |
| 74 | Burkholderia | FTLTDIVKMASNNGGAQALKAVLEHGPTLDERG | NN |
| 75 | Burkholderia | FSAADIVKIAGNSGGAQALQAVISHRAALTQAG | NS |
| 76 | Burkholderia | FSGGDAVSTVVRSGGAQSVASGGTASGTTVSAG | RS |
| 77 | Burkholderia | FRQTDIVKMAGSGGSAQALNAVIKHGPTLRQRG | SG |
| 78 | Burkholderia | FSLIDIVEIASNGGAQALKAVLKYGPVLIQAGR | SN |
| 79 | Burkholderia | FSGGDAAGTVVSSGGAQNVIGGLASGTTVASGG | SS |
| 80 | Paraburkholderia | FNLTDIVEMAANSGGAQALKAVLEHGPTLRQRG | NS |
| 81 | Paraburkholderia | FNRASIVKIAGNSGGAQALQAVLKHGPTLDERG | NS |
| 82 | Paraburkholderia | FSQANIVKMAGNSGGAQALQAVLDLELVFRERG | NS |
| 83 | Paraburkholderia | FSQPDIVKMAGNSGGAQALQAVLDLELAFRERG | NS |
| 84 | Paraburkholderia | FSLIDIVEIASNGGAQALKAVLKYGPVLMQAGR | SN |
| 85 | Francisella | YKSEDIIRLASHDGGSVNLEAVLRLHSQLTRLG | HD |
| 86 | Francisella | YKPEDIIRLASHGGGSVNLEAVLRLNPQLIGLG | HG |
| 87 | Francisella | YKSEDIIRLASHGGGSVNLEAVLRLHSQLTRLG | HG |
| 88 | Francisella | YKSEDIIRLASHGGGSVNLEAVLRLNPQLIGLG | HG |
| 89 | L. quateirensis | LGHKELIKIAARNGGGNNLIAVLSCYAKLKEMG | RN |
| 114 | Paraburkholderia | FNLTDIVEMAGKGGGAQALKAVLEHGPTLRQRG | KG |
| 115 | Paraburkholderia | FRQADIIKIAGNDGGAQALQAVIEHGPTLRQHG | ND |
| 116 | Paraburkholderia | FSQADIVKIAGNDGGTQALHAVLDLERMLGERG | ND |
| 117 | Paraburkholderia | FSRADIVKIAGNGGGAQALKAVLEHEATLDERG | NG |
| 118 | Paraburkholderia | FSRADIVRIAGNGGGAQALYSVLDVEPTLGKRG | NG |
| 119 | Paraburkholderia | FSQPDIVKMASNIGGAQALQAVLELEPALRERG | NI |
| 120 | Paraburkholderia | FSQPDIVKMAGNIGGAQALQAVLSLGPALRERG | NI |
| 121 | Paraburkholderia | FSQPEIVKIAGNIGGAQALHTVLELEPTLHKRG | NI |
| 122 | Paraburkholderia | FSQSDIVKIAGNIGGAQALQAVLDLESMLGKRG | NI |
| 123 | Paraburkholderia | FSQSDIVKIAGNIGGAQALQAVLELEPTLRESD | NI |
| 124 | Paraburkholderia | FNPTDIVKIAGNKGGAQALQAVLELEPALRERG | NK |
| 125 | Paraburkholderia | FSPTDIIKIAGNNGGAQALQAVLDLELMLRERG | NN |
| 126 | Paraburkholderia | FSQADIVKIAGNNGGAQALYSVLDVEPTLGKRG | NN |
| 127 | Paraburkholderia | FSRGDIVTIAGNNGGAQALQAVLELEPTLRERG | NN |
| 128 | Paraburkholderia | FSRIDIVKIAANNGGAQALHAVLDLGPTLRECG | NN |
| 129 | Paraburkholderia | FSQADIVKIVGNNGGAQALQAVFELEPTLRERG | NN |
| 130 | Paraburkholderia | FSQPDIVRITGNRGGAQALQAVLALELTLRERG | NR |
| 131 | Legionellales | FKADDAVRIACRIGGSHNLKAVHKNYERLRARG | RT |
| 132 | Legionellales | FNADQVIKIVGHDGGSNNIDVVQQFFPELKAFG | HD |
| 133 | Legionella | FSAEQIVRIAAHIGGSRNIEATIKHYAMLIQPP | HI |
| | maceachernii | | |
| 134 | Francisella | YKSEDIIRLASHDGGSVNLEAVLRLNPQLIGLG | HD |
| 135 | Francisella | YKSEDIIRLASHDGGSINLEAVLRLNPQLIGLG | HD |
| 136 | Francisella | YKSEDIIRLASSNGGSVNLEAVLRLNPQLIGLG | SN |
| 137 | Francisella | YKSEDIIRLASSNGGSVNLEAVIAVHKALHSNG | SN |
| 138 | Legionellales | FSADQVVKIAGHSGGSNNIAVMLAVFPRLRDFG | HS |
| 151 | Francisella | YKINHCVNLLKLNHDGFMLKNLIPYDSKLIGLG | LN |
| 152 | Francisella | YNKKQIVLIASGSSGG | GS |
| | (half-repeat) | | |

In any one of the animal pathogen-derived repeat domains of SEQ ID NOs: 2-10, 23-89, and 114-137 there can be considerable sequence divergence between repeats of a MAP-NBD in addition to the sequence variation of the BCR. This lack of conservation of sequence outside of the 12th and 13th amino acid positions in the RUs described herein contrasts with TALE proteins that include repeats in which the sequence outside of the 12th and 13th amino acid positions is mostly conserved.

In some embodiments, a MAP-NBD of the present disclosure can comprise between 1 to 50 animal pathogen-derived repeat domains, e.g., between 9 and 36, between 12 and 30, between 5 to 10, between 10 to 15, between 15 to 20, between 20 to 25, between 25 to 30, between 30 to 35 animal pathogen-derived repeat domains, or between 35 to 40 animal pathogen-derived repeat domains. In certain aspects, a MAP-NBD described herein can comprise 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 animal pathogen-derived repeat domains.

An animal pathogen-derived repeat domain can be derived from a wild-type repeat domain, such as any one of SEQ ID NOs: 2-10, 23-89, 114-138, and 151-152. An animal pathogen-derived repeat domain can also comprise a modified animal pathogen-derived repeat domain enhanced for specific recognition of a nucleotide or base pair. A MAP-NBD described herein can comprise one or more wild-type animal pathogen-derived repeat domains, one or more modified animal pathogen-derived repeat domains, or a combination thereof. In some embodiments, a modified animal pathogen-derived repeat domain can comprise 1, 2, 3, 4, 5, 6, 7, 8, 9, 10, 11, 12, 13, 14, 15, 16, 17, 18, 19, 20, 21, 22, 23, 24, 25, 26, 27, 28, or 29 mutations that can enhance recognition of a specific nucleobase or base pair. In some embodiments, a modified animal pathogen-derived repeat domain can comprise more than 1 modification, for example 1 to 5 modifications, 5 to 10 modifications, 10 to 15 modifications, 15 to 20 modifications, 20 to 25 modifications, or 25-29 modifications. In some embodiments, a MAP-NBD can comprise more than one modified animal pathogen-derived repeat domains, wherein each of the modified animal pathogen-derived repeat domains can have a different number of modifications.

In some embodiments, a MAP-NBD of the present disclosure can have the full length naturally occurring N-terminus of a naturally occurring L. quateirensis-derived protein, such as the N-terminus of SEQ ID NO: 1. An N-terminus can be the full-length N-terminus sequence and can have a sequence: MPDLELNFAIPLHLFDDETVFTHDATNDNSQASSSYSSKSSPASANARKRTSRKEMSGPPSK EPANTKSRRANSQNNKLSLADRLTKYNIDEEFYQTRSDSLLSLNYTKKQIERLILYKGRTSA VQQLLCKHEELLNLISPDG (SEQ ID NO: 13). In some embodiments, any truncation of SEQ ID NO: 13 can be used as the N-terminus in a MAP-NBD of the present disclosure. For example, in some embodiments, a MAP-NBD comprises a truncated N-terminus including amino acid residues at position 15 (D) to position 143 (G) of the naturally occurring L. quateirensis N-terminus as follows: DATNDNSQASSSYSSKSSPASANARKRTSRKEMSGPPSKEPANTKSRRANSQNNKLSLADR LTKYNIDEEFYQTRSDSLLSLNYTKKQIERLILYKGRTSAVQQLLCKHEELLNLISPDG (SEQ ID NO: 20). In some embodiments, a MAP-NBD comprises a truncated N-terminus including amino acid residues at position 29 (N) to position 143 (G) of the naturally occurring L. quateirensis N-terminus as follows: NSQASSSYSSKSSPASANARKRTSRKEMSGPPSKEPANTKSRRANSQNNKLSLADRLTKYNI DEEFYQTRSDSLLSLNYTKKQIERLILYKGRTSAVQQLLCKHEELLNLISPDG (SEQ ID NO: 21). In some embodiments, any truncation of the naturally occurring L. quateirensis N-terminus can be used at the N-terminus of a NBD disclosed herein. The naturally occurring N-terminus of L. quateirensis can be truncated to amino acid residues at positions 1 to 50, 1 to 70, 1 to 100, 1 to 120, 1 to 130, 10 to 40, 60 to 100, or 100 to 120 and used at the N-terminus of the MAP-NBD.

In some embodiments, the present disclosure provides methods for identifying an animal pathogen-derived repeat domain. For example, a consensus sequence can be defined comprising a first repeat motif, a spacer, and a second repeat motif. The consensus sequence can be 1xxx211x1xxx33x2x1xxxxxxxxx1xxxx1xxx211x1xxx33x2x1xxxxxxxxx1, 1xxx211x1xxx33x2x1xxxxxxxxx1xxxxxxxx211x1xxx33x2x1xxxxxxxxx1, 1xxx211x1xxx33x2x1xxxxxxxxxxxxxxxx211x1xxx33x2x1xxxxxxxxx1, 1xxx211x1xxx33x2x1xxxxxx1xxxxxxx1xxx211x1xxx33x2x1xxxxxxxxx1, 1xxx211x1xxx33x2x1xxxxxxx1xx xxxx1xxx211x1xxx33x2x1xxxxxxxxx1. x can be any amino acid residue, 1, 2, and 3 are flexible residues that are defined as follows: 1 can be selected from any one of A, F, I, L, M, T, or V, 2 can be selected from any one of D, E, K, N, M, S, R, or Q, and 3 can be selected from any one of A, G, N, or S. Thus, in some embodiments, a MAP-NBD can be derived from an animal pathogen comprising the consensus sequence. Any one of consensus sequences can be compared against all sequences in databases such as NCBI, MGRast, JGI, and EBI to identify matches corresponding to animal pathogen proteins containing repeat units of a DNA-binding repeat domain.

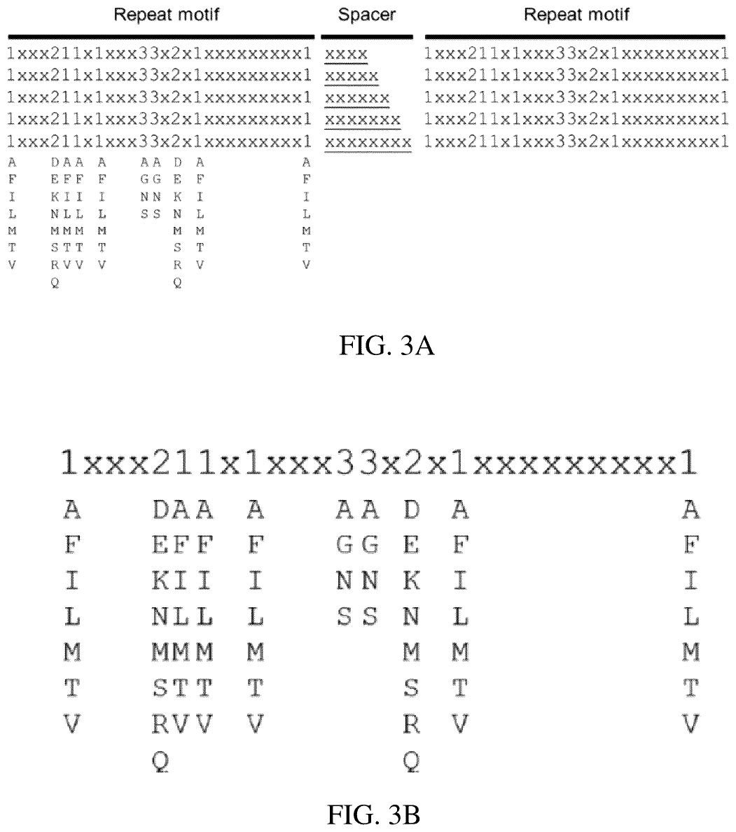

In some embodiments, a MAP-NBD repeat domain can itself have a consensus sequence of 1xxx211x1xxx33x2x1xxxxxxxxx1 (SEQ ID NO: 19), wherein x can be any amino acid residue, 1, 2, and 3 are flexible residues that are defined as follows: 1 can be selected from any one of A, F, I, L, M, T, or V, 2 can be selected from any one of D, E, K, N, M, S, R, or Q, and 3 can be selected from any one of A, G, N, or S. FIGS. 3A and 3B show consensus sequences present in an animal pathogen protein of the present disclosure and a consensus sequence in a MAP-NBD of the present disclosure. FIG. 3A shows consensus sequences that can be present in an animal pathogen-derived protein from which a MAP-NBD of the present disclosure is provided. Each consensus sequence includes a repeat motif, followed by a spacer of variable length, and a second repeat motif. FIG. 3B shows a consensus sequence which can be present in a repeat domain (e.g., a MAP-NBD) of the present disclosure.

The terms “MAP-NBD” and “NBD” are used herein interchangeably to refer to NBDs that include RUs having sequences derived from RUs identified in proteins from animal pathogens, such as, bacterium of the order Legionellales or the species Legionella or Francisella.

In certain aspects, the present disclosure provides a recombinant polypeptide comprising a nucleic acid binding domain (NBD) and a heterologous functional domain, the NBD comprising at least three repeat units (RUs) ordered from N-terminus to C-terminus of the NBD to specifically bind to a target nucleic acid, each of the RUs of the NBD comprising the consensus sequence: 1xxxx11x12xx33xxx1xxxxxxxxxx14xxx, where 1=A, F, I, L, M, T, V, or Y; 2=x or xx; 3=A, G, N, or S; 4=x, xx, or xxx; and x=any amino acid, and where each of the RUs independently comprises a 33-36 amino acid long sequence that is at least 70% (e.g., at least 75%, 80%, 85%, 90%, 95%, or 99%, or a 100%) identical to the amino acid sequence set forth in one of SEQ ID NOs: 2-9, 23-35, 85-89, and 131-137, where SEQ ID NOs: 2-9, 33, and 89 provide amino acid sequences of repeat units identified in a protein (SEQ ID NO:1) from a L. quateirensis bacterium, where SEQ ID NOs: 23-32, 34-35, and 133 provide amino acid sequences of repeat units identified in a protein (SEQ ID NO: 143) from a L. maceachernii bacterium, where SEQ ID NOs: 25, 131-132, and 138 provide amino acid sequences of repeat units identified in a protein (SEQ ID NO: 139) from a bacterium of the order Legionellales, and where SEQ ID NOs: 85-88, 134-137 provide amino acid sequences of repeat units identified in a protein (SEQ ID NO: 147) from a bacterium of the genus Francisella. In certain aspects, a half-RU is present at the C-terminus of the NBD (i.e., following the last RU), wherein the half-RU comprises a 15-20 amino acid long sequence that is at least 70% (e.g., at least 75%, 80%, 85%, 90%, 95%, or 99%, or a 100%) identical to FNAEQIVRMVSHKGGSKNL (SEQ ID NO: 10) and wherein the BCR (i.e., the amino acids at positions 12 and 13) present in the half-RU may be HK, HD, HA, HN, HG, NN, NG, RN, HI, HV, RT, HD, SN, HS, GS, or LN.

In certain aspects, the present disclosure provides a recombinant polypeptide comprising a nucleic acid binding domain (NBD) and a heterologous functional domain, the NBD comprising at least three repeat units (RUs) ordered from N-terminus to C-terminus of the NBD to specifically bind to a target nucleic acid, each of the RUs of the NBD comprising the consensus sequence: (F/L/Y)(D/G/N/S)(A/H/R/S/T/V)(D/E/K/Q)(E/H/Q)(I/L/V)(I/L/V)(C/H/K/R/S)(I/M/V)(A/V)(A/G/S)(H/N/R)(A/D/G/I/K/N/S/V)(G)(G)(A/G/S)(H/K/L/N/R)(N)(I/L)(A/D/E/I/K/V)(A/L/V)(I/M/V)(K/L/Q/T)(A/D/E/K/L/Q/S)(A/C/F/N/V/Y)(F/H/L/Q/Y)(A/D/H/P/ Q)(A/D/I/K/R/T/V)(F/L)(K/M/Q/R/S)(D/E/N/S)(F/L/M)(D/E/G/H/K/N), where the consensus sequence is based upon the amino acid sequences of repeat units identified in proteins from a bacterium of the order Legionellales, a L. quateirensis bacterium, and a L. maceachernii bacterium and having the sequences set forth in SEQ ID NOs: 2-10, 23-35, 85-89, and 131-137.

In certain aspects, the NBD provided herein may additionally include a half-RU at the C-terminus, where the half-RU comprises a 15-20 amino acid long sequence that is at least 70% (e.g., at least 75%, 80%, 85%, 90%, 95%, or 99%, or a 100%) identical to FNAEQIVRMVSX₁₂X₁₃GGSKNL (SEQ ID NO:155), or comprises a sequence having the sequence of SEQ ID NO:155 with one or more conservative amino acid substitutions thereto, and where X₁₂X₁₃=HK, HD, HA, HN, HG, NN, NG, RN, HI, HV, RT, HD, SN, HS, GS, or LN.

In certain aspects, each of the at least three RUs present in the NBD provided herein independently comprises a 33-36 amino acid long sequence that is at least 70% identical (e.g., at least 75%, 80%, 85%, 90%, 95%, or 99%, or a 100% identical) to

(SEQ ID NO: 2)
FSSQQIIRMVSHAGGANNLKAVIANHDDLQNMG;
(SEQ ID NO: 3)
FNVEQIVRMVSHNGGSKNLKAVTDNHDDLKNMG;
(SEQ ID NO: 4)
FNAEQIVRMVSHGGGSKNLKAVTDNHDDLKNMG;
(SEQ ID NO: 5)
FNAEQIVSMVSNNGGSKNLKAVTDNHDDLKNMG;
(SEQ ID NO: 6)
FNAEQIVSMVSNGGGSLNLKAVKKYHDALKDRG;
(SEQ ID NO: 7)
FNTEQIVRMVSHDGGSLNLKAVKKYHDALRERK;
(SEQ ID NO: 8)
FNVEQIVSIVSHGGGSLNLKAVKKYHDVLKDRE;
(SEQ ID NO: 9)
FNAEQIVRMVSHDGGSLNLKAVTDNHDDLKNMG;
(SEQ ID NO: 33)
FNAEQIVRMVSHKGGSKNLALVKEYFPVFSSFH; or
(SEQ ID NO: 89)
LGHKELIKIAARNGGGNNLIAVLSCYAKLKEMG;

and
comprises BCR selected from HK, HD, HA, HN, HG, NN, NG, RN, HI, HV, RT, HD, SN, HS, GS, or LN, at amino acid positions 12 and 13, respectively, numbered relative to one of SEQ ID NOs: 2-9, 33, and 89. In certain aspects, the RUs present in the NBD may have an amino acid sequence of any one of SEQ ID NOs: 2-9, 33, and 89 with one or more conservative amino acid substitutions thereto. In certain aspects, the BCR present at amino acid positions 12 and 13 of a RU disclosed herein can be substituted with another BCR or a RVD to change the base to which the RU binds.

In certain aspects, the NBD may comprise at least three of the RUs and a half-repeat unit as disclosed herein. In certain aspects, the half-repeat unit may be present as the last repeat (i.e., the last RU that binds to the last base of the target nucleic acid sequence) in the NBD. In certain aspects, a half-RU is present at the C-terminus of the NBD (i.e., following the last RU), wherein the half-RU comprises a 15-20 amino acid long sequence that is at least 70% (e.g., at least 75%, 80%, 85%, 90%, 95%, or 99%, or a 100%) identical to FNAEQIVRMVSHKGGSKNL (SEQ ID NO:10) and wherein the BCR (i.e., the amino acids at positions 12 and 13) present in the half-RU may be HK, HD, HA, HN, HG, NN, NG, RN, HI, HV, RT, HD, SN, HS, GS, or LN.

In certain aspects, the NBD may include at least one RU that binds to adenine and comprises a 33-36 amino acid long sequence that is at least 70% (e.g., at least 75%, 80%, 85%, 90%, 95%, or 99%, or a 100%) identical to FSSQQIIRMVSX₁₂X₁₃GGANNLKAVTANHDDLONMG (SEQ ID NO: 2) or comprises an amino acid sequence of SEQ ID NO:2 with one or more conservative amino acid substitutions thereto, and where X₁₂X₁₃=HA.

In certain aspects, the first RU in the NBD (i.e., the first RU that binds to the first base of the target nucleic acid sequence) may be 33-36 amino acid long sequence that is at least 70% identical (e.g., at least 75%, 80%, 85%, 90%, 95%, or 99%, or a 100% identical) to LGHKELIKIAARNGGGNNLIAVLSCYAKLKEMG (SEQ ID NO:89). In certain aspects, the BCR (i.e., the amino acids at position 12 and 13, numbered relative to SEQ ID NO:89) in the first RU of the NBD may be one of HK, HD, HA, HN, HG, NN, NG, RN, HI, HV, RT, HD, SN, HS, GS, or LN.

In certain aspects, the polypeptide comprises an N-terminal domain, where the C-terminus (i.e., the last amino acid) of the N-terminal domain is covalently linked to the N-terminus (i.e., the first amino acid) of the first RU of the NBD either directly or via a linker. In certain aspects, the N-terminal domain is the N-terminus of L. quateirensis protein having the amino acid sequence set forth in SEQ ID NO:1 and may have the amino acid sequence set forth in SEQ ID NO: 13. In certain aspects, the N-terminal domain comprises a fragment of SEQ ID NO:13, such as a fragment having the amino acid sequence set forth in SEQ ID NO: 20 or 21. In certain aspects, the N-terminal domain is a polypeptide that includes the amino acid sequence of SEQ ID NOs: 13, 20, or 21 with one or more conservative amino acid substitutions thereto. In certain aspects, the N-terminal domain is a polypeptide that includes an amino acid sequence at least 85% (e.g., at least 90%, 95%, or 99%, or a 100%) identical to the amino acid sequence of any one of SEQ ID NOs: 13, 20, or 21. In certain aspects, the N-terminal domain is a N-cap domain or a fragment thereof from TALE proteins like those expressed in Burkholderia, Paraburkholderia, or Xanthomonas.

In certain aspects, the polypeptide comprises a C-terminal domain, where the N-terminus (i.e., the first amino acid) of the C-terminal domain is covalently linked to the C-terminus (i.e., the last amino acid) of the last RU or the half-repeat unit, if present, in the NBD either directly or via a linker. In certain aspects, the C-terminal domain is the C-terminus of L. quateirensis protein having the amino acid sequence set forth in SEQ ID NO: 1 and may have the amino acid sequence set forth in SEQ ID NO:12 or SEQ ID NO: 159. In certain aspects, the C-terminal domain comprises a fragment of SEQ ID NO:12, such as a fragment having the amino acid sequence set forth in SEQ ID NO: 22. In certain aspects, the C-terminal domain is a polypeptide that includes the amino acid sequence of SEQ ID NO:12, 159, or 22 with one or more conservative amino acid substitutions thereto. In certain aspects, the C-terminal domain is a polypeptide that includes an amino acid sequence at least 85% (e.g., at least 90%, 95%, or 99%, or a 100%) identical to the amino acid sequence of any one of SEQ ID NO: 12, 159, or 22. In certain aspects, the C-terminal domain is a C-cap domain or a fragment thereof from TALE proteins like those expressed in Burkholderia, Paraburkholderia, or Xanthomonas.

In certain aspects, each of the three or more RUs present in the NBD of the polypeptides disclosed herein independently comprises a 33-36 amino acid long sequence that is at least 70% (e.g., at least 75%, 80%, 85%, 90%, 95%, or 99%, or a 100%) identical to:

(SEQ ID NO: 131)
FKADDAVRIACRIGGSHNLKAVHKNYERLRARG;
(SEQ ID NO: 132)
FNADQVIKIVGHDGGSNNIDVVQQFFPELKAFG;
(SEQ ID NO: 138)
FSADQVVKIAGHSGGSNNIAVMLAVFPRLRDFG; or
(SEQ ID NO: 25)
LDRQQILRIASHDGGSKNIAAVQKFLPKLMNFG,

and may include BCR selected from HK, HD, HA, HN, HG, NN, NG, RN, HI, HV, RT, HD, SN, HS, GS, or LN, at amino acid positions 12 and 13, respectively, numbered relative to one of SEQ ID NOs: 25, 131, 132, and 138. In certain aspects, each of the three or more RUs may independently have the sequence of one of SEQ ID NOs: 25, 131, 132, and 138 with one or more conservative amino acid substitutions thereto. As indicated in Table 1, SEQ ID NOs: 25, 131, 132, and 138 are repeat units identified in a protein from a bacterium of the order Legionellales. This Legionellales bacterium protein has the amino acid sequence:

(SEQ ID NO: 139)
MPKTKITTVSHGYDLDLMSSLPNGDPNQAKQGKIYLSGNGVYVVRDVAGI
VHRGQLEFAINLEQLEQKINEPAFKAVILEKTSRAVGYTISNECENVELN
ALAKAGENNLDIDKLIFRRSSRGTVQTVLNSYNILLEKPYNLDRQQILRI
ASHDGGSKNIAAVQKFLPKLMNFGFNADQVIKIVGHDGGSNNIDVVQQFF
PELKAFGFSADQVVKIAGHSGGSNNIAVMLAVFPRLRDFGFKADDAVRIA
CRIGGSHNLKAVHKNYERLRARGYDNKKIISIAASNCGTETINTIMSTDE
VEESDFLYFVTTVSTPVASQNLSSASNININYSNRFMTARKKTSDDNTDE
VEEDQHRDKRRSNGR

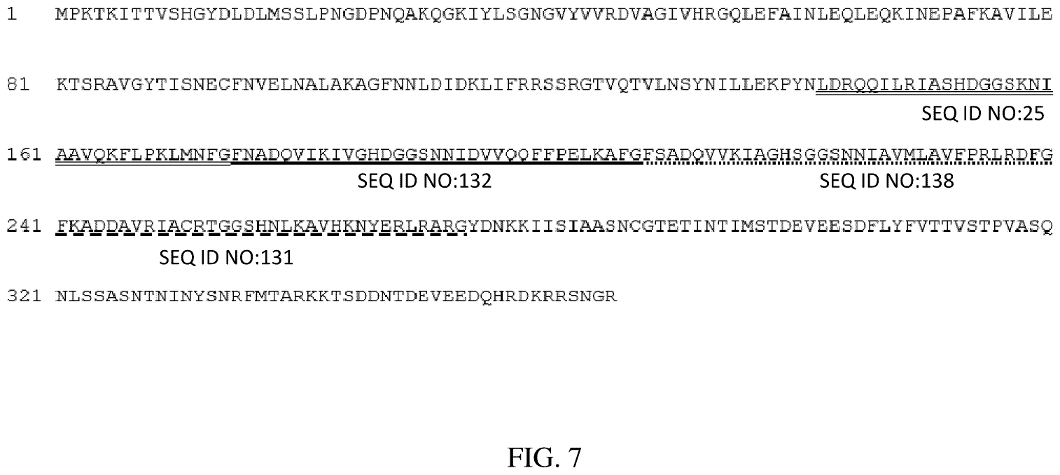

The RUs identified in this protein are indicated by SEQ ID NOs: 25, 131, 132, and 138 in FIG. 7.

In certain aspects, the polypeptide comprises an N-terminal domain, where the C-terminus (i.e., the last amino acid) of the N-terminal domain is covalently linked to the N-terminus (i.e., the first amino acid) of the first RU of the NBD either directly or via a linker. In certain aspects, the N-terminal domain comprises an amino acid sequence at least 85% (e.g., at least 90%, 95%, or 99%, or a 100%) identical to the amino acid sequence set forth in SEQ ID NO:140 or a fragment thereof. SEQ ID NO:140 sets forth the amino acid sequence N-terminal to the repeat of SEQ ID NO: 25 as shown in FIG. 7. A fragment of SEQ ID NO:140 may exclude about 10, 20, or 30 amino acids from the N-terminus of SEQ ID NO:140. In certain aspects, the N-terminal domain is a polypeptide that includes the amino acid sequence of SEQ ID NOs: 13, 20, 21, 140 with one or more conservative amino acid substitutions thereto. In certain aspects, the N-terminal domain is a polypeptide that includes an amino acid sequence at least 85% (e.g., at least 90%, 95%, or 99%, or a 100%) identical to the amino acid sequence of any one of SEQ ID NOs: 13, 20, 21, or 140. In certain aspects, the N-terminal domain is a N-cap domain or a fragment thereof from TALE proteins like those expressed in Burkholderia, Paraburkholderia, or Xanthomonas.

In certain aspects, the polypeptide comprises a C-terminal domain, where the N-terminus (i.e., the first amino acid) of the C-terminal domain is fused to the C-terminus (i.e., the last amino acid) of the last RU or the half-repeat unit, if present, in the NBD either directly or via a linker. In certain aspects, the C-terminal domain is the C-terminus of the Legionellales bacterium protein having the amino acid sequence set forth in SEQ ID NO:139 and may have the amino acid sequence set forth in SEQ ID NO:141. SEQ ID NO:141 sets forth the amino acid sequence C-terminal to the last repeat of SEQ ID NO: 131 as shown in FIG. 7. A fragment of SEQ ID NO:141 may exclude about 10, 20, or 25 amino acids from the C-terminus of SEQ ID NO:141. In certain aspects, the C-terminal domain comprises a fragment of SEQ ID NO:141, such as a fragment having the amino acid sequence set forth in SEQ ID NO: 142. In certain aspects, the C-terminal domain is a polypeptide that includes the amino acid sequence of SEQ ID NO:141 or 142 with one or more conservative amino acid substitutions thereto. In certain aspects, the C-terminal domain is a polypeptide that includes an amino acid sequence at least 85% (e.g., at least 90%, 95%, or 99%, or a 100%) identical to the amino acid sequence of any one of SEQ ID NO:141 or 142. In certain aspects, the C-terminal domain is a C-cap domain or a fragment thereof from TALE proteins like those expressed in Burkholderia, Paraburkholderia, or Xanthomonas.

In certain aspects, the C-terminal domain is a polypeptide that includes the amino acid sequence of SEQ ID NO:12, 159, 22, 141, or 142 with one or more conservative amino acid substitutions thereto. In certain aspects, the C-terminal domain is a polypeptide that includes an amino acid sequence at least 85% (e.g., at least 90%, 95%, or 99%, or a 100%) identical to the amino acid sequence of any one of SEQ ID NO:12, 159, 22, 141, or 142. In certain aspects, the C-terminal domain is a C-cap domain or a fragment thereof from TALE proteins like those expressed in Burkholderia, Paraburkholderia, or Xanthomonas.

In certain aspects, each of the three or more RUs present in the NBD of the polypeptides disclosed herein independently comprises a 33-36 amino acid long sequence that is at least 70% (e.g., at least 75%, 80%, 85%, 90%, 95%, or 99%, or a 100%) identical to:

(SEQ ID NO: 23)
FSAEQIVRIAAHDGGSRNIEAVQQAQHVLKELG;
(SEQ ID NO: 24)
FSAEQIVSIVAHDGGSRNIEAVQQAQHILKELG;
(SEQ ID NO: 26)
FSAEQIVRIAAHDGGSLNIDAVQQAQQALKELG;
(SEQ ID NO: 27)
FSTEQIVCIAGHGGGSLNIKAVLLAQQALKDLG;
(SEQ ID NO: 28)
YSSEQIVRVAAHGGGSLNIKAVLQAHQALKELD;
(SEQ ID NO: 29)
FSAEQIVHIAAHGGGSLNIKAILQAHQTLKELN;
(SEQ ID NO: 30)
FSAEQIVRIAAHIGGSRNIEAIQQAHHALKELG;
(SEQ ID NO: 31)
FSAEQIVRIAAHIGGSHNLKAVLQAQQALKELD;
(SEQ ID NO: 32)
FSAKHIVRIAAHIGGSLNIKAVQQAQQALKELG;
(SEQ ID NO: 34)
FSADQIVRIAAHKGGSHNIVAVQQAQQALKELD;
(SEQ ID NO: 35)
FSAEQIVSIAAHVGGSHNIEAVQKAHQALKELD; or
(SEQ ID NO: 133)
FSAEQIVRIAAHIGGSRNIEATIKHYAMLTQPP,

and
comprises BCR selected from HK, HD, HA, HN, HG, NN, NG, RN, HI, HV, RT, HD, SN, HS, GS, or LN at amino acid positions 12 and 13, respectively, numbered relative to one of SEQ ID NOs: 23-24, 26-32, 34-35, and 133. In certain aspects, each of the three or more RUs may independently have the sequence of one of SEQ ID NOs: 23-24, 26-32, 34-35, and 133 with one or more conservative amino acid substitutions thereto.

The RUs listed in SEQ ID NOs: 23-24, 26-32, 34-35, and 133 were identified in a protein from L. maceachernii. The amino acid sequence for this protein is set forth in SEQ ID NO:143:

MPKINQPKNLEAKSTKNKISLPQDPQTLNELKIKGYPQDLAERLIKKGSS
LAVKTVLKDHEQLVNFFTHLQIIRMAAQKGGAKNITTALNEYNSLINLGY
SSEQIVRVAAHGGGSLNIKAVLQAHQALKELDESAEQIVRIAAHDGGSLN
IDAVQQAQQALKELGFSAKHIVRIAAHIGGSLNIKAVQQAQQALKELGFS
ADQIVRIAAHKGGSHNIVAVQQAQQALKELDFSAEQIVRIAAHIGGSRNI
EAIQQAHHALKELGFSAEQIVRIAAHIGGSHNLKAVLQAQQALKELDFSA
EQIVRIAAHDGGSRNIEAVQQAQHVLKELGFSAEQIVHIAAHGGGSLNIK
AILQAHQTLKELNFSAEQIVSIVAHDGGSRNIEAVQQAQHILKELGFSTE
QIVCIAGHGGGSLNIKAVLLAQQALKDLGFSAEQIVSIAAHVGGSHNIEA
VQKAHQALKELDESAEQIVRIAAHIGGSRNIEATIKHYAMLTQPPYMLSQ
EQFLRLIDHHSGHLNLSILLDEQQWQAINDLCLQPHHFGRQNALEKFLQQ
GQRKYQNLQELEQFLFQDSADPMLLQETENQHEAEKINDCMDFILRLISA
TEPLDLQIEIEGIGLFSPSMHFDATQANFSTPAANEEKIDNSATEAGVNS
RKRKIAAAHQKQPPRKKTATPLSATFISTLITLAQSDNPRLEMASAEALM
LKAPQKLAMGITVRKKTKCEGIAIITVTDKTKLNGWLSSASESTYSSVEA
QGTRIVNNTHAFFSTPLISDKKSPSFSSLDFYEDSGLGFDEEITNPPYMP
ELEPEFIL

The 12 RUs identified in the protein from L. maceachernii are indicated by underlining and include the amino acid sequences set forth in SEQ ID NOs: 28, 26, 32, 34, 30, 31, 23, 29, 24, 27, 35, and 133, respectively.

In certain aspects, the polypeptide comprises an N-terminal domain, where the C-terminus of the N-terminal domain is fused to the N-terminus of the first RU of the NBD either directly or via a linker. In certain aspects, the N-terminal domain comprises an amino acid sequence at least 85% (e.g., at least 90%, 95%, or 99%, or a 100%) identical to the amino acid sequence set forth in SEQ ID NO: 144 or a fragment thereof. SEQ ID NO:144 sets forth the amino acid sequence N-terminal to the repeat of SEQ ID NO: 28 in SEQ ID NO:143 and has the following sequence:

(SEQ ID NO: 144)
MPKTNQPKNLEAKSTKNKISLPQDPQTLNELKIKGYPQDLAERLIKKGSS
LAVKTVLKDHEQLVNFFTHLQIIRMAAQKGGAKNITTALNEYNSLTNLG

A fragment of SEQ ID NO:144 may exclude about 10, 20, or 30 amino acids from the N-terminus of SEQ ID NO:144. In certain aspects, the N-terminal domain is a polypeptide that includes the amino acid sequence of SEQ ID NOs: 13, 20, 21, 140, or 144 with one or more conservative amino acid substitutions thereto. In certain aspects, the N-terminal domain is a polypeptide that includes an amino acid sequence at least 85% (e.g., at least 90%, 95%, or 99%, or a 100%) identical to the amino acid sequence of any one of SEQ ID NOs: 13, 20, 21, 140, or 144. In certain aspects, the N-terminal domain is a N-cap domain or a fragment thereof from TALE proteins like those expressed in Burkholderia, Paraburkholderia, or Xanthomonas.

In certain aspects, the polypeptide comprises a C-terminal domain, where the N-terminus of the C-terminal domain is fused to the C-terminus of the last RU or the half-repeat unit, if present, in the NBD either directly or via a linker. In certain aspects, the C-terminal domain is the C-terminus of the L. maceachernii protein having the amino acid sequence set forth in SEQ ID NO:143 and may have the amino acid sequence set forth in SEQ ID NO:145. SEQ ID NO:145 sets forth the amino acid sequence C-terminal to the last repeat of SEQ ID NO: 133 in SEQ ID NO:143 and has the following sequence:

(SEQ ID NO: 145)
YMLSQEQFLRLIDHHSGHLNLSILLDEQQWQAINDLCLQPHHFGRQNALE
KFLQQGQRKYQNLQELEQFLFQDSADPMLLQETENQHEAEKINDCMDFIL
RLISATEPLDLQIEIEGIGLFSPSMHFDATQANFSTPAANEEKIDNSATE
AGVNSRKRKIAAAHQKQPPRKKTATPLSATFISTLTTLAQSDNPRLEMAS
AEALMLKAPQKLAMGITVRKKTKCEGIAIITVTDKTKLNGWLSSASESTY
SSVEAQGTRTVNNTHAFFSTPLTSDKKSPSFSSLDFYEDSGLGFDEEITN
PPYMPELEPEFIL.

A fragment of SEQ ID NO:145 may exclude about 10, 20, or 25 amino acids from the C-terminus of SEQ ID NO:145. In certain aspects, the C-terminal domain comprises a fragment of SEQ ID NO:145, such as a fragment having the amino acid sequence set forth in SEQ ID NO: 146. In certain aspects, the C-terminal domain is a polypeptide that includes the amino acid sequence of SEQ ID NO:145 or 146 with one or more conservative amino acid substitutions thereto. In certain aspects, the C-terminal domain is a polypeptide that includes an amino acid sequence at least 85% identical to the amino acid sequence of any one of SEQ ID NO:145 or 146. In certain aspects, the C-terminal domain is a C-cap domain or a fragment thereof from TALE proteins like those expressed in Burkholderia, Paraburkholderia, or Xanthomonas.

In certain aspects, an N-terminus domain or a C-terminus domain derived from the L. maceachernii protein having the sequence set forth in SEQ ID NO:143 may be used to construct a polypeptide comprising a NBD, wherein the N-terminus domain mediates binding to the sequence G or G-G. In addition to including one or both of the N-terminus domain and the C-terminus domain derived from the L. maceachernii protein having the sequence set forth in SEQ ID NO:143, the polypeptide may include a plurality of repeats, where each repeat has a sequence derived independently from one or more of SEQ ID NOs: 28, 26, 32, 34, 30, 31, 23, 29, 24, 27, 35, and 133, for binding the bases T, C, A, G, G, A or C, C, T, C, T, G or A, and A or G, respectively.

In certain aspects, a recombinant polypeptide comprising a NBD and a heterologous functional domain, the NBD comprising at least three repeat units (RUs) ordered from N-terminus to C-terminus of the NBD to specifically bind to a target nucleic acid, where each of the RUs comprises the consensus sequence: YK(P/S)EDIIRLASH(D/G)GGSVNLEAVLRL(H/N)(P/S)QL(I/T)(G/R)LG (SEQ ID NO:156) is provided. This consensus sequence is based on the repeats identified in a Francisella bacterium protein having the amino acid sequence:

(SEQ ID NO: 147)
MTLTLKQEKIAINKKLRSYRISKRKFLLDFSKMNLSPEGLNYAQEELAKL
QFQAKASLETDQGINIEEQLYQLGYTQSHLRPCADRYNCSILLNILLINN
NSFVIQEISLENRVNLVVAANGNNDGIQVFFKTYPKLKSVGYKINHCVNL
LKLNHDGFMLKNLIPYDSKLIGLGYKPEDIIRLASHGGGSVNLEAVLRLN
PQLIGLGYKSEDIIRLASHDGGSVNLEAVLRLHSQLIRLGYKSEDIIRLA
SHGGGSVNLEAVLRLHSQLIRLGYKSEDIIRLASHGGGSVNLEAVLRLNP
QLIGLGYKSEDIIRLASHGGGSVNLEAVLRLHSQLTRLGYKSEDIIRLAS
HGGGSVNLEAVLRLNPQLIGLGYKSEDIIRLASHGGGSVNLEAVLRLHSQ
LTRLGYKSEDIIRLASHGGGSVNLEAVLRLNPQLIGLGYKSEDIIRLASH
DGGSVNLEAVLRLNPQLIGLGYKSEDIIRLASHDGGSINLEAVLRLNPQL
IGLGYKSEDIIRLASSNGGSVNLEAVLRLNPQLIGLGYKSEDIIRLASSN
GGSVNLEAVLRLNPQLIGLGYKSEDIIRLASHDGGSVNLEAVLRLHSQLT
RLGYKSEDIIRLASSNGGSVNLEAVIAVHKALHSNGYNKKQIVLIASGSS
GGMRLHALNNYHFLFHSIENIQLLITILQSHLEAFRIEQYMISGVLLNLL
KQGQVISEQPCKILIDSSILNPNICQTLSNIINKYQFKNKPLYFNPTTSI
ITCMLSTQECYQLLAVWERRNISPSEILNNLLNPINIFQYQLISQTNEPD
VYFLDCYHWHKFYPNMEIKQLQQLLIKAINLGINNCDILPEDNRILIIEP
YNDNWIKLSISIIDTIMDDSFNNLTRELFFCQLAPDSSNLIDDAIYIYKT
QQTIEFLVISKSRSSERFILDISTIYKDTIEEIEQALTHKLGALKGATYH
TLIKCLLAQGYQVIGYFSMNIIGADVMPPTIIADDYPEYITLEWLSSEPM
SQRSRLRTHDINSIKTLHNPTPKSQAIHQMLNLLALPDAISPLDSIQNNH
TSANHEQQTQGRISPISQQLDITLMRSRKRPLQKSDNTIYHDKRYWTFIG
EGSYNKAYTDGQGFVVKVAKNELGLMDKSERSVRVFNEINPTLPQEVLAH
VSQDLWISPLIENETLSPIEQASFIFKTYIEHGRLILDGYCQNNLLQSAK
YNTPVCIDPGNVVRRNSIASQEHWYAANEKILLRRQLYRKHMIDTIDHYH
KIRHIDRILPILMILALDFIDRKMQHLQLQLILKKNIKSLGIAFYFYYKH
NQSSTQQEFILSANIIDKILYGDQYICDILDQSFKTLNKSRVVILFRQIN
IDMSLI

The RUs present in SEQ ID NO: 147 have amino acid sequences as set forth in SEQ ID NOS: 85-87, 134-137, and 151 and the half-RU present in SEQ ID NO:147 has an amino acid sequence as set forth in SEQ ID NO:152. These RUs are indicated by underlining in SEQ ID NO:147 above. The RU having the amino acid sequence set forth in SEQ ID NO:151 occurs once in SEQ ID NO:147. The RU having the amino acid sequence set forth in SEQ ID NO: 86 occurs once in SEQ ID NO: 147. SEQ ID NO: 85 occurs twice in SEQ ID NO:147. The RU having the amino acid sequence set forth in SEQ ID NO: 87 occurs thrice in SEQ ID NO:147. The RU having the amino acid sequence set forth in SEQ ID NO: 88 occurs thrice in SEQ ID NO:147. The RU having the amino acid sequence set forth in SEQ ID NO:134 occurs once in SEQ ID NO:147. The RU having the amino acid sequence set forth in SEQ ID NO:135 occurs twice in SEQ ID NO:147. The RU having the amino acid sequence set forth in SEQ ID NO: 136 occurs twice in SEQ ID NO:147.

In certain aspects, the NBD of the recombinant polypeptide disclosed herein may include at three RUs, where each RU independently comprises a 33-36 amino acid long sequence that is at least 70% (e.g., at least 75%, 80%, 85%, 90%, 95%, or 99%, or a 100%) identical to:

(SEQ ID NO: 85)
YKSEDI IRLASHDGGSVNLEAVLRLHSQLTRLG;
(SEQ ID NO: 86)
YKPEDIIRLASHGGGSVNLEAVLRLNPQLIGLG;
(SEQ ID NO: 87)
YKSEDIIRLASHGGGSVNLEAVLRLHSQLTRLG;
(SEQ ID NO: 88)
YKSEDIIRLASHGGGSVNLEAVLRLNPQLIGLG;
(SEQ ID NO: 134)
YKSEDIIRLASHDGGSVNLEAVLRLNPQLIGLG;
(SEQ ID NO: 135)
YKSEDIIRLASHDGGSINLEAVLRLNPQLIGLG;
(SEQ ID NO: 136)
YKSEDIIRLASSNGGSVNLEAVLRLNPQLIGLG;
(SEQ ID NO: 137)
YKSEDIIRLASSNGGSVNLEAVIAVHKALHSNG,
or
(SEQ ID NO: 151)
YKINHCVNLLKLNHDGFMLKNLIPYDSKLTGLG;

and
comprises BCR selected from HK, HD, HA, HN, HG, NN, NG, RN, HI, HV, RT, HD, SN, HS, GS, or LN at amino acid positions 12 and 13, respectively, numbered relative to one of SEQ ID NOs: 85-88,134-137, and 151. In certain aspects, each of the RUs may independently have an amino acid sequence of any one of SEQ ID NOs: 85-88, 134-137, and 151 with one or more conservative amino acid substitutions thereto.

In certain aspects, the polypeptide comprises a half-RU comprises a 15-20 amino acid long sequence that is at least 70% (e.g., at least 75%, 80%, 85%, 90%, 95%, or 99%, or a 100%) identical to the amino acid sequence set forth in SEQ ID NO: 152 (YNKKQIVLIASGSSGG). In certain aspects, the half-RU may include BCR selected from HK, HD, HA, HN, HG, NN, NG, RN, HI, HV, RT, HD, SN, HS, GS, or LN at amino acid positions 12 and 13, respectively, numbered relative to SEQ ID NO: 152.

In certain aspects, the polypeptide comprises an N-terminal domain, wherein the C-terminus of the N-terminal domain is fused to the N-terminus of the first RU of the NBD directly or via a linker. In certain aspects, the N-terminal domain comprises an amino acid sequence at least 85% identical to the amino acid sequence set forth in SEQ ID NO:148 or a fragment thereof. SEQ ID NO: 148 is the amino acid sequence N-terminus to the first RU of SEQ ID NO:86 present in SEQ ID NO: 147. In certain aspects, the N-terminal domain comprises an amino acid sequence at least 85% (e.g., at least 90%, 95%, or 99%, or a 100%) identical to the amino acid sequence set forth in SEQ ID NOs: 13, 20, 21, 140, or 144.

In certain aspects, the polypeptide comprises C-terminal domain, wherein the N-terminus of the C-terminal domain is fused to the C-terminus of the last RU of the NBD directly or via a linker. In certain aspects, the C-terminal domain comprises an amino acid sequence at least 85% (e.g., at least 90%, 95%, or 99%, or a 100%) identical to the amino acid sequence set forth in SEQ ID NO: 149 or a fragment thereof, e.g., SEQ ID NO:147. In certain aspects, the C-terminal domain is a polypeptide that includes an amino acid sequence at least 85% (e.g., at least 90%, 95%, or 99%, or a 100%) identical to the amino acid sequence of any one of SEQ ID NO:12, 159, 22, 141, 142, 145, or 146. In certain aspects, the C-terminal domain is a C-cap domain or a fragment thereof from TALE proteins like those expressed in Burkholderia, Paraburkholderia, or Xanthomonas.

In certain aspects, the N-terminal domains may be derived from the N-terminal regions, e.g., N-cap domain used in conjunction with DNA binding domains disclosed in US20180010152. In certain aspects, the N-terminal domains may be derived from the N-terminal regions disclosed in US20150225465, e.g., SEQ ID NOs.: 7, 8, or 9 disclosed therein.

In certain aspects, the RUs present in a NBD of the present disclosure can be independently selected from the RUs provided herein. In certain aspects, the individual RUs listed in Table 1 can be used to generate a NBD without any amino acid modification (e.g., deletion, insertion, and/or substitution). In certain aspects, the individual RUs listed in Table 1 can be modified via deletion, insertion, and/or substitution and used to generate a NBD. As noted herein, RUs identified herein from different animal pathogen proteins can be mixed and matched to create a NBD. In other aspects, a NBD may include RUs from only one genus of bacteria as disclosed herein. In certain aspects, the NBD provided herein does not include the RUs in an order that occurs in the naturally occurring protein from which it is derived, for example, in a protein from a Legionella species bacterium, a Francisella species bacterium, or a Legionellales order bacterium. In certain aspects, the NBD disclosed herein include a half-repeat at the C-terminus of the NBD, where the half-repeat forms the last RU in the NBD before the start of a linker and/or a C-domain as provided herein.

In certain aspects, one or more RUs in a NBD provided herein may be at least 70%, 75%, 80%, 85%, 90%, 91%, 92%, 93%, 94%, 95%, 96%, 97%, 98%, or 99%, or a 100% identical to a RU provided herein. Percent identity between a pair of sequences may be calculated by multiplying the number of matches in the pair by 100 and dividing by the length of the aligned region, including gaps. Identity scoring only counts perfect matches and does not consider the degree of similarity of amino acids to one another. Only internal gaps are included in the length, not gaps at the sequence ends.

$$Percent Identity = ( Matches \times 100 ) / Length of aligned region ( with gaps )$$

The phrase “conservative amino acid substitution” refers to substitution of amino acid residues within the following groups: 1) L, I, M, V, F; 2)R, K; 3) F, Y, H, W, R; 4) G, A, T, S; 5) Q, N; and 6) D, E. Conservative amino acid substitutions may preserve the activity of the protein by replacing an amino acid(s) in the protein with an amino acid with a side chain of similar acidity, basicity, charge, polarity, or size of the side chain.

Guidance for substitutions, insertions, or deletions may be based on alignments of amino acid sequences of proteins from different species or from a consensus sequence based on a plurality of proteins having the same or similar function.

In certain aspects, the N-terminal domain or the C-terminal domain included in the disclosed NBD may include a nuclear localization sequence (NLS) to facilitate entry into the nucleus of a cell, e.g., an animal or a plant cell. In certain aspects, the polypeptide may be produced in a host cell and expressed with a translocation signal at the N-terminus which translocation signal may be cleaved during translocation.

In certain aspects, the RUs may be linked C-terminus to N-terminus with no additional amino acids separating immediately adjacent RUs. In certain aspects, immediately adjacent RUs may be separated by a spacer sequence of at least one amino acid. In certain aspects, the spacer sequence includes at least 2, 3, 4, 5, 6, or 7 amino acids, or up to 5, or up to 10 amino acids. The spacer sequence may include amino acids that have small side chains. In certain aspects, the spacer sequence is a flexible linker.

### Linkers

Any functional domain, such as a nuclease domain, a gene regulation domain, or a fluorophore can be linked to a NBD as provided herein, e.g., a MAP-NBD (e.g., a L. quateirensis, Burkholderia, Paraburkholderia, or Francisella-derived) either directly or through a linker. The linker can be naturally occurring or non-naturally occurring (e.g., synthetic). In some embodiments, a MAP-NBD (e.g., a L. quateirensis, Burkholderia, Paraburkholderia, or Francisella-derived) can be produced with a “molecular velcro” at its N or C-terminus. For example, the MAP-NBD (e.g., a L. quateirensis, Legionellales, Burkholderia, Paraburkholderia, or Francisella-derived) can be produced with a first peptide sequence. Separately, a nuclease of interest (e.g., a half cleavage domain such as FokI) can be engineered with a second peptide sequence that is complementary and binds to the first peptide sequence, thereby allowing the formation of a MAP-NBD-nuclease after administration in cells.

In other embodiments, a MAP-NBD (e.g., a L. quateirensis, Burkholderia, Paraburkholderia, or Francisella-derived) can be produced with a linker at its N- or C-terminus and subsequently directly conjugated to a functional domain (e.g., gene editing domain, gene regulator domain, or fluorescent domain).

In certain aspects, a linker can be encoded by a nucleic acid sequence that is 3 to 540 nucleotides in length, e.g., 3 to 18, 18 to 33, 33 to 48, 48 to 63, 63 to 78, 78 to 93, 93 to 108, 108 to 123, 123 to 138, 138 to 153, 153 to 168, 168 to 183, 183 to 198, 198 to 213, 213 to 228, 228 to 243, 243 to 258, 258 to 273, 273 to 288, 288 to 303, 303 to 318, 318 to 333, 333 to 348, 348 to 363, 363 to 378, 378 to 393, 393 to 408, 408 to 423, 423 to 438, 438 to 453, 453 to 468, 468 to 483, 483 to 498, 498 to 513, 513 to 528, 528 to 543, 3, 18, 33, 48, 63, 78, 93, 108, 123, 138, 153, 168, 183, 198, 213, 228, 243, 258, 273, 288, 303, 318, 333, 348, 363, 378, 393, 408, 423, 438, 453, 468, 483, 498, 513, 528, or 543 nucleotides in length. In certain aspects, a linker can be encoded by a nucleic acid sequence that is 3, 18, 33, 48, 63, 78, 93, 108, 123, 138, 153, 168, 183, 198, 213, 228, 243, 258, 273, 288, 303, 318, 333, 348, 363, 378, 393, 408, 423, 438, 453, 468, 483, 498, 513, 528, or 543 nucleotides in length.

In certain aspects, a linker can be from 1 to 180, from 1 to 20, from 20 to 40, from 40 to 60, from 60 to 80, from 80 to 100, from 100 to 120, from 120 to 140, from 140 to 160, or from 160 to 180 amino acid residues in length.

A linker for linking a nuclease domain to a MAP-NBD (e.g., L. quateirensis, Burkholderia, Paraburkholderia, or Francisella-derived) can be 1, 10, 20, 30, 40, 50, 60, 70, 80, 90, 100, 110, 120, 130, 140, 150, 160, 170, or 180 amino acid residues in length. A linker can be 1, 10, 20, 30, 40, 50, 60, 70, 80, 90, 100, 110, 120, 130, 140, 150, 160, 170, or 180 amino acid residues in length.

In certain aspects, the linker can be the C-terminus of a naturally occurring L. quateirensis-derived protein, such as the C-terminus of SEQ ID NO: 1. A C-terminus can be the full-length C-terminus sequence and can have a sequence of ALVKEYFPVFSSFHFTADQIVALICQSKQCFRNLKKNHQQWKNKGLSAEQIVDLILQETPPK PNFNNTSSSTPSPSAPSFFQGPSTPIPTPVLDNSPAPIFSNPVCFFSSRSENNTEQYLQDSTLDLD SQLGDPTKNFNVNNFWSLFPFDDVGYHPHSNDVGYHLHSDEESPFFDF (SEQ ID NO: 12). In some embodiments, any truncation of SEQ ID NO: 12 can be used as a linker. In some embodiments, a linker can be a C-terminus with amino acid residues at position 1 (A) to position 63 (P) of the naturally occurring L. quateirensis-derived protein as follows:

(SEQ ID NO: 22)
ALVKEYFPVFSSFHFTADQIVALICQSKQCFR
NLKKNHQQWKNKGLSAEQIVDLILQETPPKP.

In certain aspects, the linker can be the C-terminus of a naturally occurring L. quateirensis-derived protein, such as the C-terminus of SEQ ID NO: 1. A C-terminus can be the full-length C-terminus sequence and can have a sequence of

(SEQ ID NO: 12)
ALVKEYFPVFSSFHFTADQIVALICQSKQCFRNLKKNHQQWKNK
GLSAEQIVDLILQETPPKPNFNNTSSSTPSPSAPSFFQGPSTPI
PTPVLDNSPAPIFSNPVCFFSSRSENNTEQYLQDSTLDLDSQLG
DPTKNFNVNNFWSLFPFDDVGYHPHSNDVGYHLHSDEESPFFDF.

In certain aspects, the linker may be a C-terminal domain as disclosed herein.

In certain aspects, the C-terminal domains that can be present in the polypeptides disclosed herein may be derived from the C-terminal regions, e.g., C-cap domain used in conjunction with DNA binding domains disclosed in US20180010152. In certain aspects, the C-terminal domains may be derived from the C-terminal regions disclosed in US20150225465, e.g., SEQ ID NOs: 64, 65, or 66 disclosed therein.

### Functional Domains

A NBD as disclosed herein or a MAP-NBD (e.g., L. quateirensis, Burkholderia, Paraburkholderia, or Francisella-derived) can be linked to a functional domain. The functional domain can provide different types of activity, such as genome editing, gene regulation (e.g., activation or repression), or visualization of a genomic locus via imaging. In certain aspects, the functional domain is heterologous to the NBD. Heterologous in the context of a functional domain and a NBD as used herein indicates that these domains are derived from different sources and do not exist together in nature.

In certain aspects, a method of modulating expression of an endogenous gene in a cell is disclosed. The method may include introducing into the cell a polypeptide comprising a NBD and a heterologous functional domain, where the endogenous gene comprises the target nucleic acid sequence bound by the NBD of the polypeptide and the heterologous functional domain modulates expression of the endogenous gene. In some embodiments, the polypeptide can be introduced as a nucleic acid encoding the polypeptide. In certain aspects, the nucleic acid is a DNA or a RNA, e.g., a mRNA. In certain aspects, the cell is a human cell and the sequence of the nucleic acid is codon optimized for expression in a human cell. In certain aspects, the functional domain is a transcriptional activator and the target nucleic acid sequence is present in an expression control region of the gene, where the polypeptide increases expression of the gene. In certain aspects, the functional domain is a transcriptional repressor and the target nucleic acid sequence is present in an expression control region of the gene, wherein the polypeptide decreases expression of the gene. In certain aspects, the expression control region of the gene comprises a promoter region of the gene.

In some aspects, the functional domain is a nuclease comprising a cleavage domain or a half-cleavage domain and the endogenous gene is inactivated by cleavage. In some aspects, the inactivation occurs via non-homologous end joining (NHEJ). In some aspects, the inactivation occurs by deletion or insertion of base pairs, introduction of a single nucleotide polymorphism (SNP), or introduction of a longer stretch of heterologous DNA. In some aspects, the inactivation occurs by generation of a premature stop codon by cleavage, as taught herein. In some aspects, the polypeptide is a first polypeptide that binds to a first target nucleic acid sequence in the gene and comprises a half-cleavage domain and the method comprises introducing a second polypeptide that is a polypeptide comprising a NBD that binds to a second target nucleic acid sequence in the gene and comprises a half-cleavage domain. In some aspects, the first target nucleic acid sequence and the second target sequence are spaced apart in the gene and the two half-cleavage domains mediate a cleavage of the gene sequence at a location in between the first and second target nucleic acid sequences.

Also provided herein is a method of introducing an exogenous nucleic acid into a region of interest in the genome of a cell, the method comprising: introducing into the cell: (i) a polypeptide comprising a NBD as disclosed and a cleavage domain or a half cleavage domain, where NBD of the polypeptide binds to a target nucleic acid sequence present adjacent the region of interest, and (ii) the exogenous nucleic acid, where the cleavage domain or the half-cleavage domain introduces a cleavage in the region of interest and where the exogenous nucleic acid in integrated into the cleaved region of interest by homologous recombination.

Further aspects of the polypeptides and methods are described below.

### A. Genome Editing Domains

A NBD as disclosed herein or a MAP-NBD (e.g., L. quateirensis, Burkholderia, Paraburkholderia, or Francisella-derived) can be linked to a nuclease, wherein the MAP-NBD provides specificity and targeting and the nuclease provides genome editing functionality. In some embodiments, the nuclease can be a cleavage half domain, which dimerizes to form an active full domain capable of cleaving DNA. In other embodiments, the nuclease can be a cleavage domain, which is capable of cleaving DNA without needing to dimerize. For example, a nuclease comprising a cleavage half domain can be an endonuclease, such as FokI or Bfil. In some embodiments, two cleavage half domains (e.g., FokI or Bfil) can be fused together to form a fully functional single cleavage domain. When half cleavage domains are used as the nuclease, two MAP-NBDs can be engineered, the first MAP-NBD binding to a top strand of a target nucleic acid sequence and comprising a first FokI cleavage half domain and a second MAP-NBD binding to a bottom strand of a target nucleic acid sequence and comprising a second FokI half cleavage domain. In some embodiments, the nuclease can be a type IIS restriction enzyme, such as FokI or Bfil.

In some embodiments, a cleavage domain capable of cleaving DNA without need to dimerize may be a meganuclease. Meganucleases are also referred to as homing endonucleases. In some embodiments, the meganuclease may be I-AniI or I-OnuI.

A nuclease domain fused to a MAP-NBD (e.g., L. quateirensis, Burkholderia, Paraburkholderia, or Francisella-derived) can be an endonuclease or an exonuclease. An endonuclease can include restriction endonucleases and homing endonucleases. An endonuclease can also include S1 Nuclease, mung bean nuclease, pancreatic DNase I, micrococcal nuclease, or yeast HO endonuclease. An exonuclease can include a 3′-5′ exonuclease or a 5′-3′ exonuclease. An exonuclease can also include a DNA exonuclease or an RNA exonuclease. Examples of exonuclease includes exonucleases I, II, III, IV, V, and VIII; DNA polymerase I, RNA exonuclease 2, and the like.

A nuclease domain fused to a NBD as disclosed herein or a MAP-NBD (e.g., L. quateirensis, Burkholderia, Paraburkholderia, or Francisella-derived) can be a restriction endonuclease (or restriction enzyme). In some instances, a restriction enzyme cleaves DNA at a site removed from the recognition site and has a separate binding and cleavage domains. In some instances, such a restriction enzyme is a Type IIS restriction enzyme.

A nuclease domain fused to a NBD as disclosed herein or a MAP-NBD (e.g., L. quateirensis, Burkholderia, Paraburkholderia, or Francisella-derived) can be a Type IIS nuclease. A Type IIS nuclease can be FokI or Bfil. In some cases, a nuclease domain fused to a MAP-NBD (e.g., L. quateirensis, Burkholderia, Paraburkholderia, or Francisella-derived) is FokI. In other cases, a nuclease domain fused to a MAP-NBD (e.g., L. quateirensis, Burkholderia, Paraburkholderia, or Francisella-derived) is Bfil.

FokI can be a wild-type FokI or can comprise one or more mutations. In some cases, FokI can comprise 1, 2, 3, 4, 5, 6, 7, 8, 9, 10, or more mutations. A mutation can enhance cleavage efficiency. A mutation can abolish cleavage activity. In some cases, a mutation can modulate homodimerization. For example, FokI can have a mutation at one or more amino acid residue positions 446, 447, 479, 483, 484, 486, 487, 490, 491, 496, 498, 499, 500, 531, 534, 537, and 538 to modulate homodimerization.

In some instances, a FokI cleavage domain is, for example, as described in Kim et al. “Hybrid restriction enzymes: Zinc finger fusions to Fok I cleavage domain,” PNAS 93:1156-1160 (1996). In some cases, a FokI cleavage domain described herein is a FokI of SEQ ID NO: 11 (TABLE 2). In other instances, a FokI cleavage domain described herein is a FokI, for example, as described in U.S. Pat. No. 8,586,526.

TABLE 2 illustrates an exemplary FokI sequence that can be used herein with a method or system described herein.

TABLE 2

| SEQ ID NO | FokI Sequence |
|---|---|
| SEQ ID NO: 11 | QLVKSELEEKKSELRHKLKYVPHEYIELIEI |
| | ARNSTQDRILEMKVMEFFMKVYGYRGKHLGG |
| | SRKPDGAIYTVGSPIDYGVIVDTKAYSGGYN |
| | LPIGQADEMQRYVEENQTRNKHINPNEWWKV |
| | YPSSVTEFKFLFVSGHFKGNYKAQLTRLNHI |
| | TNCNGAVLSVEELLIGGEMIKAGTLTLEEVR |
| | RKENNGEINF |

A MAP-NBD (e.g., L. quateirensis, Burkholderia, Paraburkholderia, or Francisella-derived) can be linked to a functional group that modifies DNA nucleotides, for example an adenosine deaminase.

### B. Regulatory Domains

As another example, NBD as disclosed herein or a MAP-NBD (e.g., L. quateirensis, Burkholderia, Paraburkholderia, or Francisella-derived) can be linked to a gene regulating domain. A gene regulation domain can be an activator or a repressor. For example, a NBD as disclosed herein or a MAP-NBD (e.g., L. quateirensis, Burkholderia, Paraburkholderia, or Francisella-derived) can be linked to an activation domain, such as VP16, VP64, p65, p300 catalytic domain, TET1 catalytic domain, TDG, Ldb1 self-associated domain, SAM activator (VP64, p65, HSF1), or VPR (VP64, p65, Rta). The terms “activator,” “activation domain” and “transcriptional activator” are used interchangeably to refer to a polypeptide that increases expression of a gene. Alternatively, a MAP-NBD (e.g., L. quateirensis, Burkholderia, Paraburkholderia, or Francisella-derived) can be linked to a repressor, such as KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2. The terms “repressor,” “repressor domain,” and “transcriptional repressor” are used herein interchangeably to refer to a polypeptide that decreases expression of a gene.

In some embodiments, a NBD as disclosed herein or a MAP-NBD (e.g., L. quateirensis, Burkholderia, Paraburkholderia, or Francisella-derived) can be linked to a DNA modifying protein, such as DNMT3a. A MAP-NBD (e.g., L. quateirensis, Burkholderia, Paraburkholderia, or Francisella-derived) can be linked to a chromatin-modifying protein, such as lysine-specific histone demethylase 1 (LSD1). A MAP-NBD (e.g., L. quateirensis, Burkholderia, Paraburkholderia, or Francisella-derived) can be linked to a protein that is capable of recruiting other proteins, such as KRAB. The DNA modifying protein (e.g., DNMT3a) and proteins capable of recruiting other proteins (e.g., KRAB) can serve as repressors of transcription. Thus, MAP-NBDs (e.g., L. quateirensis, Burkholderia, Paraburkholderia, or Francisella-derived) linked to a DNA modifying protein (e.g., DNMT3a) or a domain capable of recruiting other proteins (e.g., KRAB, a domain found in transcriptional repressors, such as Kox1) can provide gene repression functionality, can serve as transcription factors, wherein the MAP-NBD (e.g., L. quateirensis, Burkholderia, Paraburkholderia, or Francisella-derived) provides specificity and targeting and the DNA modifying protein and the protein capable of recruiting other proteins provides gene repression functionality, which can be referred to as an engineered genomic regulatory complex or a MAP-NBD-gene regulator (MAP-NBD-GR) and, more specifically, as a MAP-NBD-transcription factor (MAP-NBD-TF).

In some embodiments, expression of the target gene can be reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 92%, at least 95%, at least 97%, or at least 99% by using a DNA binding domain fused to a repression domain (e.g., a MAP-NBD-TF) of the present disclosure as compared to non-treated cells. In some embodiments, expression of a checkpoint gene can be reduced by over 90% by using a MAP-NBD-TF of the present disclosure as compared to non-treated cells.

In some embodiments, repression of the target gene with a DNA binding domain fused to a repression domain (e.g., a MAP-NBD-TF) of the present disclosure and subsequent reduced expression of the target gene can last for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, or at least 28 days. In some embodiments, repression of the target gene with a MAP-NBD-TF of the present disclosure and subsequent reduced expression of the target gene can last for 1 days to 3 days, 3 days to 5 days, 5 days to 7 days, 7 days to 9 days, 9 days to 11 days, 11 days to 13 days, 13 days to 15 days, 15 days to 17 days, 17 days to 19 days, 19 days to 21 days, 21 days to 23 days, 23 days to 25 days, or 25 days to 28 days.

In various aspects, the present disclosure provides a method of identifying a target binding site in a target gene of a cell, the method comprising: (a) contacting a cell with an engineered transcriptional repressor comprising a DNA binding domain, a repressor domain, and a linker; (b) measuring expression of the target gene; and (c) determining expression of the target gene is repressed by at least 50%, at least 60%, at least 70%, at least 80%, at least 85%, at least 90%, at least 92%, at least 95%, at least 97%, or at least 99% for at least 3 days, wherein the target gene is selected from: a checkpoint gene and a T cell surface receptor.

In some aspects, expression of the target gene is repressed in at least 75%, at least 80%, at least 85%, at least 90%, at least 95%, or at least 99% of a plurality of the cells. In some aspects, the engineered genomic regulatory complex is undetectable after at least 3 days. In some aspects, determining the engineered genomic regulatory complex is undetectable is measured by qPCR, imaging of a FLAG-tag, or a combination thereof. In some aspects, the measuring expression of the target gene comprises flow cytometry quantification of expression of the target gene.

In some embodiments, repression of the target gene with a DNA binding domain fused to a repression domain (e.g., a MAP-NBD-TF) of the present disclosure can last even after the DNA binding domain-TF becomes undetectable. The DNA binding domain fused to a repression domain (e.g., a MAP-NBD-TF) can become undetectable after at least 3 days. In some embodiments, the DNA binding domain fused to a repression domain (e.g., a MAP-NBD-TF) can become undetectable after at least 1 day, at least 2 days, at least 3 days, at least 4 days, at least 5 days, at least 6 days, at least 1 week, at least 2 weeks, at least 3 weeks, or at least 4 weeks. In some embodiments, qPCR or imaging via the FLAG-tag can be used to confirm that the DNA binding domain fused to a repression domain (e.g., a MAP-NBD-TF) is no longer detectable.

In some embodiments, repression of the PDCD1 gene can be achieved using the MAP-NBDs comprising repeat units derived from L. quateirensis. The sequence of the target DNA present in the promoter region of human PDCD1 gene, the amino acid sequences of a NBD capable of binding to the target DNA and comprising either a full-length C-terminal domain from SEQ ID NO:1 or a C-terminal domain from SEQ ID NO:1 truncated at amino acid position 63 are shown in TABLE 3.

TABLE 3
MAP-NBDs Targeting the PDCD1 Gene

| Target DNA | | |
|---|---|---|
| Sequence in | | MAP-NBD with C-terminal |
| PDCD1 (5' -> 3') | Full Length MAP-NBD | truncation to C63 |
| GAGGAAGAGGG | MPDLELNFAIPLHLFDDETVFTHDAT | MPDLELNFAIPLHLFDDETVFTHDA |
| GGCGGGAG | NDNSQASSSYSSKSSPASANARKRTSR | TNDNSQASSSYSSKSSPASANARKR |
| (SEQ ID NO: 90) | KEMSGPPSKEPANTKSRRANSQNNKL | TSRKEMSGPPSKEPANTKSRRANS |
| | SLADRLTKYNIDEEFYQTRSDSLLSLN | QNNKLSLADRLTKYNIDEEFYQTRS |
| | YTKKQIERLILYKGRTSAVQQLLCKH | DSLLSLNYTKKQIERLILYKGRTSA |
| | EELLNLISPDGLGHKELIKIAARNGGG | VQQLLCKHEELLNLISPDGLGHKEL |
| | NNLIAVLSCYAKLKEMGFSSQQIIRM | IKIAARNGGGNNLIAVLSCYAKLKE |
| | VSHAGGANNLKAVTANHDDLQNMG | MGFSSQQIIRMVSHAGGANNLKAV |
| | FNVEQIVRMVSHNGGSKNLKAVTDN | TANHDDLQNMGFNVEQIVRMVSH |
| | HDDLKNMGFNVEQIVRMVSHNGGSK | NGGSKNLKAVTDNHDDLKNMGFN |
| | NLKAVTDNHDDLKNMGFSSQQIIRM | VEQIVRMVSHNGGSKNLKAVTDN |
| | VSHAGGANNLKAVTANHDDLQNMG | HDDLKNMGFSSQQIIRMVSHAGGA |
| | FSSQQIIRMVSHAGGANNLKAVTANH | NNLKAVTANHDDLQNMGFSSQQII |
| | DDLQNMGFNVEQIVRMVSHNGGSKN | RMVSHAGGANNLKAVTANHDDLQ |
| | LKAVTDNHDDLKNMGFSSQQIIRMVS | NMGFNVEQIVRMVSHNGGSKNLK |
| | HAGGANNLKAVTANHDDLQNMGEN | AVTDNHDDLKNMGFSSQQIIRMVS |
| | VEQIVRMVSHNGGSKNLKAVTDNHD | HAGGANNLKAVTANHDDLONMGF |
| | DLKNMGFNVEQIVRMVSHNGGSKNL | NVEQIVRMVSHNGGSKNLKAVTD |
| | KAVTDNHDDLKNMGFNVEQIVRMVS | NHDDLKNMGFNVEQIVRMVSHNG |
| | HNGGSKNLKAVTDNHDDLKNMGFN | GSKNLKAVTDNHDDLKNMGFNVE |
| | VEQIVRMVSHNGGSKNLKAVTDNHD | QIVRMVSHNGGSKNLKAVTDNHD |
| | DLKNMGFNVEQIVRMVSHNGGSKNL | DLKNMGFNVEQIVRMVSHNGGSK |
| | KAVTDNHDDLKNMGFNTEQIVRMVS | NLKAVTDNHDDLKNMGFNVEQIV |
| | HDGGSLNLKAVKKYHDALRERKFNV | RMVSHNGGSKNLKAVTDNHDDLK |
| | EQIVRMVSHNGGSKNLKAVTDNHDD | NMGFNTEQIVRMVSHDGGSLNLKA |
| | LKNMGFNVEQIVRMVSHNGGSKNLK | VKKYHDALRERKFNVEQIVRMVSH |
| | AVTDNHDDLKNMGFNVEQIVRMVSH | NGGSKNLKAVTDNHDDLKNMGFN |
| | NGGSKNLKAVTDNHDDLKNMGFSSQ | VEQIVRMVSHNGGSKNLKAVTDN |
| | QIIRMVSHAGGANNLKAVTANHDDL | HDDLKNMGFNVEQIVRMVSHNGG |
| | QNMGFNAEQIVRMVSHKGGSKNLAL | SKNLKAVTDNHDDLKNMGFSSQQI |
| | VKEYFPVFSSFHFTADQIVALICQSKQ | IRMVSHAGGANNLKAVTANHDDL |
| | CFRNLKKNHQQWKNKGLSAEQIVDLI | QNMGFNAEQIVRMVSHKGGSKNL |
| | LQETPPKPNFNNTSSSTPSPSAPSFFQG | ALVKEYFPVFSSFHFTADQIVALIC |
| | PSTPIPTPVLDNSPAPIFSNPVCFFSSRS | |
| | ENNTEQYLQDSTLDLDSQLGDPTKNF | QSKQCFRNLKKNHQQWKNKGLSA |
| | NVNNFWSLFPFDDVGYHPHSNDVGY | EQIVDLILQETPPKP |
| | HLHSDEESPFFDF | (SEQ ID NO: 99) |
| | (SEQ ID NO: 98) | |
| GAGGGGGCGGG | MPDLELNFAIPLHLFDDETVFTHDAT | MPDLELNFAIPLHLFDDETVFTHDA |
| AGCAAGGGG | NDNSQASSSYSSKSSPASANARKRTSR | TNDNSQASSSYSSKSSPASANARKR |
| (SEQ ID NO: 91) | KEMSGPPSKEPANTKSRRANSQNNKL | TSRKEMSGPPSKEPANTKSRRANS |
| | SLADRLTKYNIDEEFYQTRSDSLLSLN | QNNKLSLADRLTKYNIDEEFYQTRS |
| | YTKKQIERLILYKGRTSAVQQLLCKH | DSLLSLNYTKKQIERLILYKGRTSA |
| | EELLNLISPDGLGHKELIKIAARNGGG | VQQLLCKHEELLNLISPDGLGHKEL |
| | NNLIAVLSCYAKLKEMGFSSQQIIRM | IKIAARNGGGNNLIAVLSCYAKLKE |
| | VSHAGGANNLKAVTANHDDLQNMG | MGFSSQQIIRMVSHAGGANNLKAV |
| | FNVEQIVRMVSHNGGSKNLKAVTDN | TANHDDLQNMGFNVEQIVRMVSH |
| | HDDLKNMGFNVEQIVRMVSHNGGSK | NGGSKNLKAVTDNHDDLKNMGFN |
| | NLKAVTDNHDDLKNMGFNVEQIVRM | VEQIVRMVSHNGGSKNLKAVTDN |
| | VSHNGGSKNLKAVTDNHDDLKNMGF | HDDLKNMGFNVEQIVRMVSHNGG |
| | NVEQIVRMVSHNGGSKNLKAVTDNH | SKNLKAVTDNHDDLKNMGFNVEQ |
| | DDLKNMGFNVEQIVRMVSHNGGSKN | IVRMVSHNGGSKNLKAVTDNHDD |
| | LKAVTDNHDDLKNMGFNTEQIVRMV | LKNMGFNVEQIVRMVSHNGGSKN |
| | SHDGGSLNLKAVKKYHDALRERKEN | LKAVTDNHDDLKNMGFNTEQIVR |
| | VEQIVRMVSHNGGSKNLKAVTDNHD | MVSHDGGSLNLKAVKKYHDALRE |
| | DLKNMGFNVEQIVRMVSHNGGSKNL | RKFNVEQIVRMVSHNGGSKNLKAV |
| | KAVTDNHDDLKNMGFNVEQIVRMVS | TDNHDDLKNMGFNVEQIVRMVSH |
| | HNGGSKNLKAVTDNHDDLKNMGFSS | NGGSKNLKAVTDNHDDLKNMGFN |
| | QQIIRMVSHAGGANNLKAVTANHDD | VEQIVRMVSHNGGSKNLKAVTDN |
| | LQNMGFNVEQIVRMVSHNGGSKNLK | HDDLKNMGFSSQQIIRMVSHAGGA |
| | AVTDNHDDLKNMGFNTEQIVRMVSH | NNLKAVTANHDDLQNMGFNVEQI |
| | DGGSLNLKAVKKYHDALRERKFSSQ | VRMVSHNGGSKNLKAVTDNHDDL |
| | QIIRMVSHAGGANNLKAVTANHDDL | KNMGFNTEQIVRMVSHDGGSLNLK |
| | QNMGFSSQQIIRMVSHAGGANNLKA | AVKKYHDALRERKFSSQQIIRMVS |
| | VTANHDDLQNMGFNVEQIVRMVSHN | HAGGANNLKAVTANHDDLQNMGF |
| | GGSKNLKAVTDNHDDLKNMGFNVE | SSQQIIRMVSHAGGANNLKAVTAN |
| | QIVRMVSHNGGSKNLKAVTDNHDDL | HDDLQNMGFNVEQIVRMVSHNGG |
| | KNMGFNVEQIVRMVSHNGGSKNLKA | SKNLKAVTDNHDDLKNMGFNVEQ |
| | VTDNHDDLKNMGFNAEQIVRMVSHK | IVRMVSHNGGSKNLKAVTDNHDD |
| | GGSKNLALVKEYFPVFSSFHFTADQIV | LKNMGFNVEQIVRMVSHNGGSKN |
| | ALICQSKQCFRNLKKNHQQWKNKGL | LKAVTDNHDDLKNMGFNAEQIVR |
| | SAEQIVDLILQETPPKPNFNNTSSSTPS | MVSHKGGSKNLALVKEYFPVFSSF |
| | PSAPSFFQGPSTPIPTPVLDNSPAPIFSN | HFTADQIVALICQSKQCFRNLKKNH |
| | PVCFFSSRSENNTEQYLQDSTLDLDSQ | QQWKNKGLSAEQIVDLILQETPPKP |
| | LGDPTKNFNVNNFWSLFPFDDVGYHP | (SEQ ID NO: 101) |
| | HSNDVGYHLHSDEESPFFDF | |
| | (SEQ ID NO: 100) | |
| GAGCAAGGGGC | MPDLELNFAIPLHLFDDETVFTHDAT | MPDLELNFAIPLHLFDDETVFTHDA |
| GGGCACCC | NDNSQASSSYSSKSSPASANARKRTSR | TNDNSQASSSYSSKSSPASANARKR |
| (SEQ ID NO: 92) | KEMSGPPSKEPANTKSRRANSQNNKL | TSRKEMSGPPSKEPANTKSRRANS |
| | SLADRLTKYNIDEEFYQTRSDSLLSLN | QNNKLSLADRLTKYNIDEEFYQTRS |
| | YTKKQIERLILYKGRTSAVQQLLCKH | DSLLSLNYTKKQIERLILYKGRTSA |
| | EELLNLISPDGLGHKELIKIAARNGGG | VQQLLCKHEELLNLISPDGLGHKEL |
| | NNLIAVLSCYAKLKEMGFSSQQIIRM | IKIAARNGGGNNLIAVLSCYAKLKE |
| | VSHAGGANNLKAVTANHDDLQNMG | MGFSSQQIIRMVSHAGGANNLKAV |
| | FNVEQIVRMVSHNGGSKNLKAVTDN | TANHDDLQNMGFNVEQIVRMVSH |
| | HDDLKNMGFNTEQIVRMVSHDGGSL | NGGSKNLKAVTDNHDDLKNMGFN |
| | NLKAVKKYHDALRERKFSSQQIIRMV | TEQIVRMVSHDGGSLNLKAVKKYH |
| | SHAGGANNLKAVTANHDDLQNMGFS | DALRERKFSSQQIIRMVSHAGGAN |
| | SQQIIRMVSHAGGANNLKAVTANHD | NLKAVTANHDDLQNMGFSSQQIIR |
| | DLQNMGFNVEQIVRMVSHNGGSKNL | MVSHAGGANNLKAVTANHDDLQN |
| | KAVTDNHDDLKNMGFNVEQIVRMVS | MGFNVEQIVRMVSHNGGSKNLKA |
| | HNGGSKNLKAVTDNHDDLKNMGFN | VTDNHDDLKNMGFNVEQIVRMVS |
| | VEQIVRMVSHNGGSKNLKAVTDNHD | HNGGSKNLKAVTDNHDDLKNMGF |
| | DLKNMGFNVEQIVRMVSHNGGSKNL | NVEQIVRMVSHNGGSKNLKAVTD |
| | KAVTDNHDDLKNMGFNTEQIVRMVS | NHDDLKNMGFNVEQIVRMVSHNG |
| | HDGGSLNLKAVKKYHDALRERKFNV | GSKNLKAVTDNHDDLKNMGFNTE |
| | EQIVRMVSHNGGSKNLKAVTDNHDD | QIVRMVSHDGGSLNLKAVKKYHD |
| | LKNMGFNVEQIVRMVSHNGGSKNLK | ALRERKFNVEQIVRMVSHNGGSKN |
| | AVTDNHDDLKNMGFNVEQIVRMVSH | LKAVTDNHDDLKNMGFNVEQIVR |
| | NGGSKNLKAVTDNHDDLKNMGFNTE | MVSHNGGSKNLKAVTDNHDDLKN |
| | QIVRMVSHDGGSLNLKAVKKYHDAL | MGFNVEQIVRMVSHNGGSKNLKA |
| | RERKFSSQQIIRMVSHAGGANNLKAV | VTDNHDDLKNMGFNTEQIVRMVS |
| | TANHDDLQNMGENTEQIVRMVSHDG | HDGGSLNLKAVKKYHDALRERKFS |
| | GSLNLKAVKKYHDALRERKENTEQIV | SQQIIRMVSHAGGANNLKAVTANH |
| | RMVSHDGGSLNLKAVKKYHDALRER | DDLQNMGFNTEQIVRMVSHDGGSL |
| | KFNAEQIVRMVSHDGGSKNLALVK | NLKAVKKYHDALRERKFNTEQIVR |
| | EYFPVFSSFHFTADQIVALICQSKQCF | MVSHDGGSLNLKAVKKYHDALRE |
| | RNLKKNHQQWKNKGLSAEQIVDLIL | RKFNAEQIVRMVSHDGGSKNLAL |
| | QETPPKPNFNNTSSSTPSPSAPSFFQGP | VKEYFPVFSSFHFTADQIVALICQSK |
| | STPIPTPVLDNSPAPIFSNPVCFFSSRSE | QCFRNLKKNHQQWKNKGLSAEQI |
| | NNTEQYLQDSTLDLDSQLGDPTKNFN | VDLILQETPPKP |
| | VNNFWSLFPFDDVGYHPHSNDVGYH | (SEQ ID NO: 103) |
| | LHSDEESPFFDF | |
| | (SEQ ID NO: 102) | |
| | Sequence shown in bold is based on the | Sequence shown in bold is based on the |
| | sequence of SEQ ID NO: 10 in which the | sequence of SEQ ID NO: 10 in which |
| | BCR HK have been replaced with the BCR | the BCR HK have been replaced with |
| | HD. | the BCR HD. |
| GAAGGGAGGGT | MPDLELNFAIPLHLFDDETVFTHDAT | MPDLELNFAIPLHLFDDETVFTHDA |
| GCCCGCCCC | NDNSQASSSYSSKSSPASANARKRTS | TNDNSQASSSYSSKSSPASANARKR |
| (SEQ ID NO: 93) | KREMSGPPSKEPANTKSRRANSQNNKL | TSRKEMSGPPSKEPANTKSRRANS |
| | SLADRLTKYNIDEEFYQTRSDSLLSLN | QNNKLSLADRLTKYNIDEEFYQTRS |
| | YTKKQIERLILYKGRTSAVQQLLCKH | DSLLSLNYTKKQIERLILYKGRTSA |
| | EELLNLISPDGLGHKELIKIAARNGGG | VQQLLCKHEELLNLISPDGLGHKEL |
| | NNLIAVLSCYAKLKEMGFSSQQIIRM | IKIAARNGGGNNLIAVLSCYAKLKE |
| | VSHAGGANNLKAVTANHDDLQNMG | MGFSSQQIIRMVSHAGGANNLKAV |
| | FSSQQIIRMVSHAGGANNLKAVTANH | TANHDDLQNMGFSSQQIIRMVSHA |
| | DDLQNMGFNVEQIVRMVSHNGGSKN | GGANNLKAVTANHDDLQNMGFNV |
| | LKAVTDNHDDLKNMGFNVEQIVRMV | EQIVRMVSHNGGSKNLKAVTDNH |
| | SHNGGSKNLKAVTDNHDDLKNMGFN | DDLKNMGFNVEQIVRMVSHNGGS |
| | VEQIVRMVSHNGGSKNLKAVTDNHD | KNLKAVTDNHDDLKNMGFNVEQI |
| | DLKNMGFSSQQIIRMVSHAGGANNLK | VRMVSHNGGSKNLKAVTDNHDDL |
| | AVTANHDDLQNMGFNVEQIVRMVSH | KNMGFSSQQIIRMVSHAGGANNLK |
| | NGGSKNLKAVTDNHDDLKNMGFNV | AVTANHDDLQNMGFNVEQIVRMV |
| | EQIVRMVSHNGGSKNLKAVTDNHDD | SHNGGSKNLKAVTDNHDDLKNMG |
| | LKNMGFNVEQIVRMVSHNGGSKNLK | FNVEQIVRMVSHNGGSKNLKAVTD |
| | AVTDNHDDLKNMGFNAEQIVSMVSN | NHDDLKNMGFNVEQIVRMVSHNG |
| | GGGSLNLKAVKKYHDALKDRGFNVE | GSKNLKAVTDNHDDLKNMGFNAE |
| | QIVRMVSHNGGSKNLKAVTDNHDDL | QIVSMVSNGGGSLNLKAVKKYHD |
| | KNMGFNTEQIVRMVSHDGGSLNLKA | ALKDRGFNVEQIVRMVSHNGGSKN |
| | VKKYHDALRERKFNTEQIVRMVSHD | LKAVTDNHDDLKNMGFNTEQIVR |
| | GGSLNLKAVKKYHDALRERKENTEQI | MVSHDGGSLNLKAVKKYHDALRE |
| | VRMVSHDGGSLNLKAVKKYHDALRE | RKFNTEQIVRMVSHDGGSLNLKAV |
| | RKFNVEQIVRMVSHNGGSKNLKAVT | KKYHDALRERKFNTEQIVRMVSHD |
| | DNHDDLKNMGFNTEQIVRMVSHDGG | GGSLNLKAVKKYHDALRERKENV |
| | SLNLKAVKKYHDALRERKENTEQIVR | EQIVRMVSHNGGSKNLKAVTDNH |
| | MVSHDGGSLNLKAVKKYHDALRERK | DDLKNMGFNTEQIVRMVSHDGGSL |
| | FNTEQIVRMVSHDGGSLNLKAVKKY | NLKAVKKYHDALRERKFNTEQIVR |
| | HDALRERKFNAEQIVRMVSHDGGSKN | MVSHDGGSLNLKAVKKYHDALRE |
| | LALVKEYFPVFSSFHFTADQIVALIC | RKFNTEQIVRMVSHDGGSLNLKAV |
| | QSKQCFRNLKKNHQQWKNKGLSAEQ | KKYHDALRERKFNAEQIVRMVSHD |
| | IVDLILQETPPKPNFNNTSSSTPSPSAPS | GGSKNLALVKEYFPVFSSFHFTA |
| | FFQGPSTPIPTPVLDNSPAPIFSNPVCFF | DQIVALICQSKQCFRNLKKNHQQW |
| | SSRSENNTEQYLQDSTLDLDSQLGDP | KNKGLSAEQIVDLILQETPPKP |
| | TKNFNVNNFWSLFPFDDVGYHPHSN | (SEQ ID NO: 105) |
| | DVGYHLHSDEESPFFDF | |
| | (SEQ ID NO: 104) | Sequence shown in bold is based on the |
| | | sequence of SEQ ID NO: 10 in which |
| | Sequence shown in bold is based on the | the BCR HK have been replaced with |
| | sequence of SEQ ID NO: 10 in which the | the BCR HD. |
| | BCR HK have been replaced with the BCR | |
| | HD. | |
| GACCTGGGACAG | MPDLELNFAIPLHLFDDETVFTHDAT | MPDLELNFAIPLHLFDDETVFTHDA |
| TTTCCCTT | NDNSQASSSYSSKSSPASANARKRTSR | TNDNSQASSSYSSKSSPASANARKR |
| (SEQ ID NO: 94) | KEMSGPPSKEPANTKSRRANSQNNKL | TSRKEMSGPPSKEPANTKSRRANS |
| | SLADRLTKYNIDEEFYQTRSDSLLSLN | QNNKLSLADRLTKYNIDEEFYQTRS |
| | YTKKQIERLILYKGRTSAVQQLLCKH | DSLLSLNYTKKQIERLILYKGRTSA |
| | EELLNLISPDGLGHKELIKIAARNGGG | VQQLLCKHEELLNLISPDGLGHKEL |
| | NNLIAVLSCYAKLKEMGFSSQQIIRM | IKIAARNGGGNNLIAVLSCYAKLKE |
| | VSHAGGANNLKAVTANHDDLQNMG | MGFSSQQIIRMVSHAGGANNLKAV |
| | FNTEQIVRMVSHDGGSLNLKAVKKY | TANHDDLQNMGENTEQIVRMVSH |
| | HDALRERKFNTEQIVRMVSHDGGSLN | DGGSLNLKAVKKYHDALRERKEN |
| | LKAVKKYHDALRERKFNAEQIVSMV | TEQIVRMVSHDGGSLNLKAVKKYH |
| | SNGGGSLNLKAVKKYHDALKDRGFN | DALRERKFNAEQIVSMVSNGGGSL |
| | VEQIVRMVSHNGGSKNLKAVTDNHD | NLKAVKKYHDALKDRGFNVEQIV |
| | DLKNMGFNVEQIVRMVSHNGGSKNL | RMVSHNGGSKNLKAVTDNHDDLK |
| | KAVTDNHDDLKNMGFNVEQIVRMVS | NMGFNVEQIVRMVSHNGGSKNLK |
| | HNGGSKNLKAVTDNHDDLKNMGFSS | AVTDNHDDLKNMGFNVEQIVRMV |
| | QQIIRMVSHAGGANNLKAVTANHDD | SHNGGSKNLKAVTDNHDDLKNMG |
| | LQNMGFNTEQIVRMVSHDGGSLNLK | FSSQQIIRMVSHAGGANNLKAVTA |
| | AVKKYHDALRERKFSSQQIIRMVSHA | NHDDLQNMGENTEQIVRMVSHDG |
| | GGANNLKAVTANHDDLQNMGENVE | GSLNLKAVKKYHDALRERKFSSQQ |
| | QIVRMVSHNGGSKNLKAVTDNHDDL | IIRMVSHAGGANNLKAVTANHDDL |
| | KNMGFNAEQIVSMVSNGGGSLNLKA | QNMGFNVEQIVRMVSHNGGSKNL |
| | VKKYHDALKDRGFNAEQIVSMVSNG | KAVTDNHDDLKNMGFNAEQIVSM |
| | GGSLNLKAVKKYHDALKDRGFNAEQ | VSNGGGSLNLKAVKKYHDALKDR |
| | IVSMVSNGGGSLNLKAVKKYHDALK | GFNAEQIVSMVSNGGGSLNLKAVK |
| | DRGFNTEQIVRMVSHDGGSLNLKAV | KYHDALKDRGFNAEQIVSMVSNG |
| | KKYHDALRERKFNTEQIVRMVSHDG | GGSLNLKAVKKYHDALKDRGENT |
| | GSLNLKAVKKYHDALRERKFNTEQIV | EQIVRMVSHDGGSLNLKAVKKYH |
| | RMVSHDGGSLNLKAVKKYHDALRER | DALRERKFNTEQIVRMVSHDGGSL |
| | KFNAEQIVSMVSNGGGSLNLKAVKK | NLKAVKKYHDALRERKFNTEQIVR |
| | YHDALKDRGFNAEQIVRMVSNGGGS | MVSHDGGSLNLKAVKKYHDALRE |
| | KNLALVKEYFPVFSSFHFTADQIVALI | RKFNAEQIVSMVSNGGGSLNLKAV |
| | CQSKQCFRNLKKNHQQWKNKGLSAE | KKYHDALKDRGFNAEQIVRMVSN |
| | QIVDLILQETPPKPNFNNTSSSTPSPSA | GGGSKNLALVKEYFPVFSSFHFTAD |
| | PSFFQGPSTPIPTPVLDNSPAPIFSNPVC | QIVALICQSKQCFRNLKKNHQQWK |
| | FFSSRSENNTEQYLQDSTLDLDSQLGD | NKGLSAEQIVDLILQETPPKP |
| | PTKNFNVNNFWSLFPFDDVGYHPHSN | (SEQ ID NO: 107) |
| | DVGYHLHSDEESPFFDF | |
| | (SEQ ID NO: 106) | |
| GACAGTTTCCCT | MPDLELNFAIPLHLFDDETVFTHDAT | MPDLELNFAIPLHLFDDETVFTHDA |
| TCCGCTC | NDNSQASSSYSSKSSPASANARKRTSR | TNDNSQASSSYSSKSSPASANARKR |
| (SEQ ID NO: 95) | KEMSGPPSKEPANTKSRRANSQNNKL | TSRKEMSGPPSKEPANTKSRRANS |
| | SLADRLTKYNIDEEFYQTRSDSLLSLN | QNNKLSLADRLTKYNIDEEFYQTRS |
| | YTKKQIERLILYKGRTSAVQQLLCKH | DSLLSLNYTKKQIERLILYKGRTSA |
| | EELLNLISPDGLGHKELIKIAARNGGG | VQQLLCKHEELLNLISPDGLGHKEL |
| | NNLIAVLSCYAKLKEMGFSSQQIIRM | IKIAARNGGGNNLIAVLSCYAKLKE |
| | VSHAGGANNLKAVTANHDDLQNMG | MGFSSQQIIRMVSHAGGANNLKAV |
| | FNTEQIVRMVSHDGGSLNLKAVKKY | TANHDDLQNMGENTEQIVRMVSH |
| | HDALRERKFSSQQIIRMVSHAGGANN | DGGSLNLKAVKKYHDALRERKESS |
| | LKAVTANHDDLQNMGFNVEQIVRMV | QQIIRMVSHAGGANNLKAVTANHD |
| | SHNGGSKNLKAVTDNHDDLKNMGFN | DLQNMGFNVEQIVRMVSHNGGSK |
| | AEQIVSMVSNGGGSLNLKAVKKYHD | NLKAVTDNHDDLKNMGFNAEQIV |
| | ALKDRGFNAEQIVSMVSNGGGSLNLK | SMVSNGGGSLNLKAVKKYHDALK |
| | AVKKYHDALKDRGFNAEQIVSMVSN | DRGFNAEQIVSMVSNGGGSLNLKA |
| | GGGSLNLKAVKKYHDALKDRGENTE | VKKYHDALKDRGFNAEQIVSMVS |
| | QIVRMVSHDGGSLNLKAVKKYHDAL | NGGGSLNLKAVKKYHDALKDRGF |
| | RERKFNTEQIVRMVSHDGGSLNLKAV | NTEQIVRMVSHDGGSLNLKAVKKY |
| | KKYHDALRERKFNTEQIVRMVSHDG | HDALRERKFNTEQIVRMVSHDGGS |
| | GSLNLKAVKKYHDALRERKFNAEQIV | LNLKAVKKYHDALRERKENTEQIV |
| | SMVSNGGGSLNLKAVKKYHDALKDR | RMVSHDGGSLNLKAVKKYHDALR |
| | GFNAEQIVSMVSNGGGSLNLKAVKK | ERKFNAEQIVSMVSNGGGSLNLKA |
| | YHDALKDRGFNTEQIVRMVSHDGGS | VKKYHDALKDRGFNAEQIVSMVS |
| | LNLKAVKKYHDALRERKFNTEQIVR | NGGGSLNLKAVKKYHDALKDRGF |
| | MVSHDGGSLNLKAVKKYHDALRERK | NTEQIVRMVSHDGGSLNLKAVKKY |
| | FNVEQIVRMVSHNGGSKNLKAVTDN | HDALRERKFNTEQIVRMVSHDGGS |
| | HDDLKNMGFNTEQIVRMVSHDGGSL | LNLKAVKKYHDALRERKENVEQIV |
| | NLKAVKKYHDALRERKFNAEQIVSM | RMVSHNGGSKNLKAVTDNHDDLK |
| | VSNGGGSLNLKAVKKYHDALKDRGF | NMGFNTEQIVRMVSHDGGSLNLKA |
| | NAEQIVRMVSHDGGSKNLALVKEYF | VKKYHDALRERKFNAEQIVSMVSN |
| | PVFSSFHFTADQIVALICQSKQCFRNL | GGGSLNLKAVKKYHDALKDRGFN |
| | KKNHQQWKNKGLSAEQIVDLILQETP | AEQIVRMVSHDGGSKNLALVKEY |
| | PKPNFNNTSSSTPSPSAPSFFQGPSTPIP | FPVFSSFHFTADQIVALICQSKQCFR |
| | TPVLDNSPAPIFSNPVCFFSSRSENNTE | NLKKNHQQWKNKGLSAEQIVDLIL |
| | QYLQDSTLDLDSQLGDPTKNFNVNNF | QETPPKP |
| | WSLFPFDDVGYHPHSNDVGYHLHSD | (SEQ ID NO: 109) |
| | EESPFFDF | |
| | (SEQ ID NO: 108) | Sequence shown in bold is based on the |
| | Sequence shown in bold is based on the | sequence of SEQ ID NO: 10 in which |
| | sequence of SEQ ID NO: 10 in which the | the BCR HK have been replaced with |
| | BCR HK have been replaced with the BCR | the BCR HD. |
| | HD. | |
| GAGCAGCCCCAC | MPDLELNFAIPLHLFDDETVFTHDAT | MPDLELNFAIPLHLFDDETVFTHDA |
| CAGAGTGC | NDNSQASSSYSSKSSPASANARKRTSR | TNDNSQASSSYSSKSSPASANARKR |
| (SEQ ID NO: 96) | KEMSGPPSKEPANTKSRRANSQNNKL | TSRKEMSGPPSKEPANTKSRRANS |
| | SLADRLTKYNIDEEFYQTRSDSLLSLN | QNNKLSLADRLTKYNIDEEFYQTRS |
| | YTKKQIERLILYKGRTSAVQQLLCKH | DSLLSLNYTKKQIERLILYKGRTSA |
| | EELLNLISPDGLGHKELIKIAARNGGG | VQQLLCKHEELLNLISPDGLGHKEL |
| | NNLIAVLSCYAKLKEMGFSSQQIIRM | IKIAARNGGGNNLIAVLSCYAKLKE |
| | VSHAGGANNLKAVTANHDDLQNMG | MGFSSQQIIRMVSHAGGANNLKAV |
| | FNVEQIVRMVSHNGGSKNLKAVTDN | TANHDDLQNMGFNVEQIVRMVSH |
| | HDDLKNMGFNTEQIVRMVSHDGGSL | NGGSKNLKAVTDNHDDLKNMGFN |
| | NLKAVKKYHDALRERKFSSQQIIRMV | TEQIVRMVSHDGGSLNLKAVKKYH |
| | SHAGGANNLKAVTANHDDLQNMGF | DALRERKFSSQQIIRMVSHAGGAN |
| | NVEQIVRMVSHNGGSKNLKAVTDNH | NLKAVTANHDDLQNMGENVEQIV |
| | DDLKNMGFNTEQIVRMVSHDGGSLN | RMVSHNGGSKNLKAVTDNHDDLK |
| | LKAVKKYHDALRERKFNTEQIVRMV | NMGFNTEQIVRMVSHDGGSLNLKA |
| | SHDGGSLNLKAVKKYHDALRERKEN | VKKYHDALRERKFNTEQIVRMVSH |
| | TEQIVRMVSHDGGSLNLKAVKKYHD | DGGSLNLKAVKKYHDALRERKEN |
| | ALRERKFNTEQIVRMVSHDGGSLNLK | TEQIVRMVSHDGGSLNLKAVKKYH |
| | AVKKYHDALRERKFSSQQIIRMVSHA | DALRERKFNTEQIVRMVSHDGGSL |
| | GGANNLKAVTANHDDLQNMGENTE | NLKAVKKYHDALRERKFSSQQIIR |
| | QIVRMVSHDGGSLNLKAVKKYHDAL | MVSHAGGANNLKAVTANHDDLQN |
| | RERKFNTEQIVRMVSHDGGSLNLKAV | MGFNTEQIVRMVSHDGGSLNLKAV |
| | KKYHDALRERKFSSQQIIRMVSHAGG | KKYHDALRERKFNTEQIVRMVSHD |
| | ANNLKAVTANHDDLQNMGFNVEQIV | GGSLNLKAVKKYHDALRERKFSSQ |
| | RMVSHNGGSKNLKAVTDNHDDLKN | QIIRMVSHAGGANNLKAVTANHDD |
| | MGFSSQQIIRMVSHAGGANNLKAVTA | LQNMGFNVEQIVRMVSHNGGSKN |
| | NHDDLQNMGFNVEQIVRMVSHNGGS | LKAVTDNHDDLKNMGFSSQQIIRM |
| | KNLKAVTDNHDDLKNMGFNAEQIVS | VSHAGGANNLKAVTANHDDLQNM |
| | MVSNGGGSLNLKAVKKYHDALKDR | GFNVEQIVRMVSHNGGSKNLKAVT |
| | GFNVEQIVRMVSHNGGSKNLKAVTD | DNHDDLKNMGFNAEQIVSMVSNG |
| | NHDDLKNMGFNAEQIVRMVSHDGG | GGSLNLKAVKKYHDALKDRGFNV |
| | SKNLALVKEYFPVFSSFHFTADQIVAL | EQIVRMVSHNGGSKNLKAVTDNH |
| | ICQSKQCFRNLKKNHQQWKNKGLSA | DDLKNMGFNAEQIVRMVSHDGGS |
| | EQIVDLILQETPPKPNFNNTSSSTPSPS | KNLALVKEYFPVFSSFHFTADQIVA |
| | APSFFQGPSTPIPTPVLDNSPAPIFSNPV | LICQSKQCFRNLKKNHQQWKNKG |
| | CFFSSRSENNTEQYLQDSTLDLDSQLG | LSAEQIVDLILQETPPKP |
| | DPTKNFNVNNFWSLFPFDDVGYHPHS | (SEQ ID NO: 111) |
| | NDVGYHLHSDEESPFFDF | |
| | (SEQ ID NO: 110) | Sequence shown in bold is based on the |
| | Sequence shown in bold is based on the | sequence of SEQ ID NO: 10 in which |
| | sequence of SEQ ID NO: 10 in which the | the BCR HK have been replaced with |
| | BCR HK have been replaced with the BCR | the BCR HD. |
| | HD. | |
| GATCTGCATGCC | MPDLELNFAIPLHLFDDETVFTHDAT | MPDLELNFAIPLHLFDDETVFTHDA |
| TGGAGCAG | NDNSQASSSYSSKSSPASANARKRTSR | TNDNSQASSSYSSKSSPASANARKR |
| (SEQ ID NO: 97) | KEMSGPPSKEPANTKSRRANSQNNKL | TSRKEMSGPPSKEPANTKSRRANS |
| | SLADRLTKYNIDEEFYQTRSDSLLSLN | QNNKLSLADRLTKYNIDEEFYQTRS |
| | YTKKQIERLILYKGRTSAVQQLLCKH | DSLLSLNYTKKQIERLILYKGRTSA |
| | EELLNLISPDGLGHKELIKIAARNGGG | VQQLLCKHEELLNLISPDGLGHKEL |
| | NNLIAVLSCYAKLKEMGFSSQQIIRM | IKIAARNGGGNNLIAVLSCYAKLKE |
| | VSHAGGANNLKAVTANHDDLQNMG | MGFSSQQIIRMVSHAGGANNLKAV |
| | FNAEQIVSMVSNGGGSLNLKAVKKY | TANHDDLQNMGFNAEQIVSMVSN |
| | HDALKDRGENTEQIVRMVSHDGGSL | GGGSLNLKAVKKYHDALKDRGFN |
| | NLKAVKKYHDALRERKFNAEQIVSM | TEQIVRMVSHDGGSLNLKAVKKYH |
| | VSNGGGSLNLKAVKKYHDALKDRGF | DALRERKFNAEQIVSMVSNGGGSL |
| | NVEQIVRMVSHNGGSKNLKAVTDNH | NLKAVKKYHDALKDRGFNVEQIV |
| | DDLKNMGFNTEQIVRMVSHDGGSLN | RMVSHNGGSKNLKAVTDNHDDLK |
| | LKAVKKYHDALRERKFSSQQIIRMVS | NMGFNTEQIVRMVSHDGGSLNLKA |
| | HAGGANNLKAVTANHDDLQNMGEN | VKKYHDALRERKFSSQQIIRMVSH |
| | AEQIVSMVSNGGGSLNLKAVKKYHD | AGGANNLKAVTANHDDLQNMGEN |
| | ALKDRGFNVEQIVRMVSHNGGSKNL | AEQIVSMVSNGGGSLNLKAVKKYH |
| | KAVTDNHDDLKNMGFNTEQIVRMVS | DALKDRGFNVEQIVRMVSHNGGSK |
| | HDGGSLNLKAVKKYHDALRERKENT | NLKAVTDNHDDLKNMGENTEQIV |
| | EQIVRMVSHDGGSLNLKAVKKYHDA | RMVSHDGGSLNLKAVKKYHDALR |
| | LRERKFNAEQIVSMVSNGGGSLNLKA | ERKFNTEQIVRMVSHDGGSLNLKA |
| | VKKYHDALKDRGFNVEQIVRMVSHN | VKKYHDALRERKFNAEQIVSMVSN |
| | GGSKNLKAVTDNHDDLKNMGFNVE | GGGSLNLKAVKKYHDALKDRGFN |
| | QIVRMVSHNGGSKNLKAVTDNHDDL | VEQIVRMVSHNGGSKNLKAVTDN |
| | KNMGFSSQQIIRMVSHAGGANNLKA | HDDLKNMGFNVEQIVRMVSHNGG |
| | VTANHDDLQNMGFNVEQIVRMVSHN | SKNLKAVTDNHDDLKNMGFSSQQI |
| | GGSKNLKAVTDNHDDLKNMGENTEQ | IRMVSHAGGANNLKAVTANHDDL |
| | IVRMVSHDGGSLNLKAVKKYHDALR | QNMGFNVEQIVRMVSHNGGSKNL |
| | ERKFSSQQIIRMVSHAGGANNLKAVT | KAVTDNHDDLKNMGFNTEQIVRM |
| | ANHDDLQNMGFNAEQIVRMVSHKGG | VSHDGGSLNLKAVKKYHDALRER |
| | SKNLALVKEYFPVFSSFHFTADQIVAL | KFSSQQIIRMVSHAGGANNLKAVT |
| | ICQSKQCFRNLKKNHQQWKNKGLSA | ANHDDLQNMGFNAEQIVRMVSHK |
| | EQIVDLILQETPPKPNFNNTSSSTPSPS | GGSKNLALVKEYFPVFSSFHFTADQ |
| | APSFFQGPSTPIPTPVLDNSPAPIFSNPV | IVALICQSKQCFRNLKKNHQQWKN |
| | CFFSSRSENNTEQYLQDSTLDLDSQLG | KGLSAEQIVDLILQETPPKP |
| | DPTKNFNVNNFWSLFPFDDVGYHPHS | (SEQ ID NO: 113) |
| | NDVGYHLHSDEESPFFDF | |
| | (SEQ ID NO: 112) | |

### C. Imaging Moieties

A MAP-NBD (e.g., L. quateirensis, Burkholderia, Paraburkholderia, or Francisella-derived) can be linked to a fluorophore, such as Hydroxycoumarin, methoxycoumarin, Alexa fluor, aminocoumarin, Cy2, FAM, Alexa fluor 488, Fluorescein FITC, Alexa fluor 430, Alexa fluor 532, HEX, Cy3, TRITC, Alexa fluor 546, Alexa fluor 555, R-phycoerythrin (PE), Rhodamine Red-X, Tamara, Cy3.5, Rox, Alexa fluor 568, Red 613, Texas Red, Alexa fluor 594, Alexa fluor 633, Allophycocyanin, Alexa fluor 633, Cy5, Alexa fluor 660, Cy5.5, TruRed, Alexa fluor 680, Cy7, GFP, or mCHERRY. A MAP-NBD (e.g., L. quateirensis, Burkholderia, Paraburkholderia, or Francisella-derived) can be linked to a biotinylation reagent.

### Targets

In some aspects, described herein include methods of modifying the genetic material of a target cell utilizing a MAP-NBD described herein. A target cell can be a eukaryotic cell or a prokaryotic cell. A target cell can be an animal cell or a plant cell. An animal cell can include a cell from a marine invertebrate, fish, insects, amphibian, reptile, or mammal. A mammalian cell can be obtained from a primate, ape, equine, bovine, porcine, canine, feline, or rodent. A mammal can be a primate, ape, dog, cat, rabbit, ferret, or the like. A rodent can be a mouse, rat, hamster, gerbil, hamster, chinchilla, or guinea pig. A bird cell can be from a canary, parakeet or parrots. A reptile cell can be from a turtle, lizard or snake. A fish cell can be from a tropical fish. For example, the fish cell can be from a zebrafish (e.g., Danio rerio). A worm cell can be from a nematode (e.g., C. elegans). An amphibian cell can be from a frog. An arthropod cell can be from a tarantula or hermit crab.

A mammalian cell can also include cells obtained from a primate (e.g., a human or a non-human primate). A mammalian cell can include an epithelial cell, connective tissue cell, hormone secreting cell, a nerve cell, a skeletal muscle cell, a blood cell, an immune system cell, or a stem cell.

Exemplary mammalian cells can include, but are not limited to, 293A cell line, 293 FT cell line, 293F cells, 293 H cells, HEK 293 cells, CHO DG44 cells, CHO-S cells, CHO-K1 cells, Expi293F™ cells, Flp-In™ T-REX™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REX™ Jurkat cell line, Per.C6 cells, T-REX™-293 cell line, T-REX™-CHO cell line, T-REX™-HeLa cell line, NC-HIMT cell line, PC12 cell line, primary cells (e.g., from a human) including primary T cells, primary hematopoietic stem cells, primary human embryonic stem cells (hESCs), and primary induced pluripotent stem cells (iPSCs).

In some embodiments, a MAP-NBD of the present disclosure can be used to modify a target cell. The target cell can itself be unmodified or modified. For example, an unmodified cell can be edited with a MAP-NBD of the present disclosure to introduce an insertion, deletion, or mutation in its genome. In some embodiments, a modified cell already having a mutation can be repaired with a MAP-NBD of the present disclosure.

In some instances, a target cell is a cell comprising one or more single nucleotide polymorphism (SNP). In some instances, a MAP-NBD-nuclease described herein is designed to target and edit a target cell comprising a SNP.

In some cases, a target cell is a cell that does not contain a modification. For example, a target cell can comprise a genome without genetic defect (e.g., without genetic mutation) and a MAP-NBD-nuclease described herein can be used to introduce a modification (e.g., a mutation) within the genome.

In some cases, a target cell is a cancerous cell. Cancer can be a solid tumor or a hematologic malignancy. The solid tumor can include a sarcoma or a carcinoma. Exemplary sarcoma target cell can include, but are not limited to, cell obtained from alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, or telangiectatic osteosarcoma.

Exemplary carcinoma target cell can include, but are not limited to, cell obtained from anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

Alternatively, the cancerous cell can comprise cells obtained from a hematologic malignancy. Hematologic malignancy can comprise a leukemia, a lymphoma, a myeloma, a non-Hodgkin's lymphoma, or a Hodgkin's lymphoma. In some cases, the hematologic malignancy can be a T-cell based hematologic malignancy. Other times, the hematologic malignancy can be a B-cell based hematologic malignancy. Exemplary B-cell based hematologic malignancy can include, but are not limited to, chronic lymphocytic leukemia (CLL), small lymphocytic lymphoma (SLL), high-risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. Exemplary T-cell based hematologic malignancy can include, but are not limited to, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some cases, a cell can be a tumor cell line. Exemplary tumor cell line can include, but are not limited to, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

In some embodiments, described herein include methods of modifying a target gene utilizing a MAP-NBD described herein. In some embodiments, genome editing can be performed by fusing a nuclease of the present disclosure with a DNA binding domain for a particular genomic locus of interest. Genetic modification can involve introducing a functional gene for therapeutic purposes, knocking out a gene for therapeutic gene, or engineering a cell ex vivo (e.g., HSCs or CAR T cells) to be administered back into a subject in need thereof. For example, the genome editing complex can have a target site within PDCD1, CTLA4, LAG3, TET2, BTLA, HAVCR2, CCR5, CXCR4, TRA, TRB, B2M, albumin, HBB, HBA1, TTR, NR3C1, CD52, erythroid specific enhancer of the BCL11A gene, CBLB, TGFBR1, SERPINA1, HBV genomic DNA in infected cells, CEP290, DMD, CFTR, IL2RG, CS-1, or any combination thereof. In some embodiments, a genome editing complex can cleave double stranded DNA at a target site in order to insert a chimeric antigen receptor (CAR), alpha-L iduronidase (IDUA), iduronate-2-sulfatase (IDS), or Factor 9 (F9). Cells, such as hematopoietic stem cells (HSCs) and T cells, can be engineered ex vivo with the genome editing complex. Alternatively, genome editing complexes can be directly administered to a subject in need thereof.

### Methods of Production of Polypeptides

In certain embodiments, the polypeptides disclosed herein, such as, the MAP-NBD is produced using a suitable method including recombinant and non-recombinant methods (e.g., chemical synthesis).

### A. Chemical Synthesis

Where a polypeptide is chemically synthesized, the synthesis may proceed via liquid-phase or solid-phase. Solid-phase peptide synthesis (SPPS) allows the incorporation of unnatural amino acids and/or peptide/protein backbone modification. Various forms of SPPS, such as Fmoc and Boc, are available for synthesizing polypeptides of the present disclosure. Details of the chemical synthesis are known in the art (e.g., Ganesan A. 2006 Mini Rev. Med. Chem. 6:3-10; and Camarero J. A. et al., 2005 Protein Pept Lett. 12:723-8).

### B. Recombinant Production

Where a polypeptide is produced using recombinant techniques, the polypeptide may be produced as an intracellular protein or as a secreted protein, using any suitable construct and any suitable host cell, which can be a prokaryotic or eukaryotic cell, such as a bacterial (e.g., E. coli) or a yeast host cell, respectively. In certain aspects, eukaryotic cells that are used as host cells for production of the polypeptides include insect cells, mammalian cells, and/or plant cells. In certain aspects, mammalian host cells are used and may include human cells (e.g., HeLa, 293, H9 and Jurkat cells); mouse cells (e.g., NIH3T3, L cells, and C127 cells); primate cells (e.g., Cos 1, Cos 7 and CV1) and hamster cells (e.g., Chinese hamster ovary (CHO) cells). In specific embodiments, the polypeptide disclosed herein are produced in CHO cells.

A variety of host-vector systems suitable for the expression of a polypeptide may be employed according to standard procedures known in the art. See, e.g., Sambrook et al., 1989 Current Protocols in Molecular Biology Cold Spring Harbor Press, New York; and Ausubel et al. 1995 Current Protocols in Molecular Biology, Eds. Wiley and Sons. Methods for introduction of genetic material into host cells include, for example, transformation, electroporation, conjugation, calcium phosphate methods and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically integrated. A variety of appropriate vectors for use in production of a polypeptide of interest are commercially available.

Vectors can provide for extrachromosomal maintenance in a host cell or can provide for integration into the host cell genome. The expression vector provides transcriptional and translational regulatory sequences and may provide for inducible or constitutive expression where the coding region is operably-linked under the transcriptional control of the transcriptional initiation region, and a transcriptional and translational termination region. In general, the transcriptional and translational regulatory sequences may include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7).

Also provided herein are nucleic acids encoding the polypeptides disclosed herein. In certain aspects, a nucleic acid encoding the polypeptides disclosed herein is operably linked to a promoter sequence that confers expression of the polypeptide. In certain aspects, the sequence of the nucleic acid is codon optimized for expression of the polypeptide in a human cell. In certain aspects, the nucleic acid is a deoxyribonucleic acid (DNA). In certain aspects, the nucleic acid is a ribonucleic acid (RNA). Also provided herein is a vector comprising the nucleic acid encoding the polypeptides for binding a target nucleic acid as described herein. In certain aspects, the vector is a viral vector.

In certain aspects, a host cell comprising the nucleic acid or the vector encoding the polypeptides disclosed herein is provided. In certain aspects, a host cell comprising the polypeptides disclosed herein is provided. In certain aspects, a host cell that expresses the polypeptide is also disclosed.

### Delivery

The polypeptides disclosed herein, compositions comprising the disclosed polypeptides, and nucleic acids encoding the disclosed polypeptides can be delivered into a target cell by any suitable means, including, for example, by injection, infection, transfection, and vesicle or liposome mediated delivery.

In certain aspects, a mRNA or a vector encoding the polypeptides disclosed herein may be injected, transfected, or introduced via viral infection into a target cell, where the cell is ex vivo or in vivo. Any vector systems may be used including, but not limited to, plasmid vectors, retroviral vectors, lentiviral vectors, adenovirus vectors, poxvirus vectors; herpesvirus vectors and adeno-associated virus vectors, etc. When two or more polypeptides according to present disclosure are introduced into the cell, the nucleic acids encoding the polypeptides may be carried on the same vector or on different vectors. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. Vectors suitable for introduction of polynucleotides as described herein include described herein include non-integrating lentivirus vectors (IDLV).

Non-viral vector delivery systems include electroporation, lipofection, microinjection, biolistics, virosomes, liposomes, immunoliposomes, polycation or lipid: nucleic acid conjugates, naked DNA, artificial virions, and agent-enhanced uptake of DNA.

Primary cells may be isolated and used ex vivo for reintroduction into the subject to be treated. Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells, neuronal stem cells, mesenchymal stem cells, muscle stem cells and skin stem cells. In certain aspects, the stem cells may be isolated from a subject to be treated or may be derived from a somatic cell of a subject to be treated using the polypeptides disclosed herein.

In certain aspects, the cells into which the polypeptides of the present disclosure or a nucleic acid encoding a polypeptide of the present disclosure may be an animal cell, e.g., from a human needing treatment. In other aspects, the cell may be a plant cell. DNA constructs may be introduced into (e.g., into the genome of) a desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using biolistic methods, such as DNA particle bombardment.

In certain aspects, the polypeptide of the present disclosure is only transiently present in a target cell. For example, the polypeptide is expressed from a nucleic acid that expressed the polypeptide for a short period of time, e.g., for up to 1 day, 3 days, 1 week, 3 weeks, or 1 month. In applications where transient expression of the polypeptide of the present disclosure is desired, adenoviral based systems may be used. Adeno-associated virus (“AAV”) vectors can also be used to transduce cells with nucleic acids encoding the polypeptide of the present disclosure, e.g., in the in vitro production of nucleic acids and peptides, and for in vivo and ex vivo gene therapy procedures. In certain aspects, recombinant adeno-associated virus vectors (rAAV) such as replication-deficient recombinant adenoviral vectors may be used for introduction of nucleic acids encoding the polypeptides disclosed herein.

In certain aspects, nucleic acids encoding the polypeptides disclosed herein can be delivered using a gene therapy vector with a high degree of specificity to a particular tissue type or cell type. A viral vector is typically modified to have specificity for a given cell type by including a sequence encoding a ligand expressed as a fusion protein with a viral coat protein on the viruses' outer surface. The ligand is chosen to have affinity for a receptor known to be present on the cell type of interest.

In certain aspects, gene therapy vectors can be delivered in vivo by administration to an individual patient. In certain aspects, administration involves systemic administration (e.g., intravenous, intraperitoneal, intramuscular, subdermal, or intracranial infusion), direct injection (e.g., intrathecal), or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector or which have been modified by expression of the polypeptide of the present disclosure encoded by the vector.

In certain aspects, the nucleic acid encoding the polypeptides provided herein may be codon optimized to enhance expression of the polypeptide in the target cell. For example, the sequence of the nucleic acid can be varied to provide codons that are known to be highly used in animal cells, such as, human cells to enhance production of the polypeptide in a human cell. For example, silent mutations may be made in the nucleotide sequence encoding a polypeptide disclosed herein for codon optimization in mammalian cells. Similar codon optimization can be used for optimal expression in other host cell systems (e.g. plant, fungal, etc.).

### Compositions

In certain aspects, the polypeptides and the nucleic acids described herein may be present in a pharmaceutical composition comprising a pharmaceutically acceptable excipient. In certain aspects, the polypeptides and the nucleic acids are present in a therapeutically effective amount in the pharmaceutical composition. A therapeutically effective amount can be determined based on an observed effectiveness of the composition. A therapeutically effective amount can be determined using assays that measure the desired effect in a cell, e.g., in a reporter cell line in which expression of a reporter is modulated in response to the polypeptides of the present disclosure. The pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein. Suitable pharmaceutically acceptable or physiologically acceptable diluents, carriers or excipients include, but are not limited to, nuclease inhibitors, protease inhibitors, a suitable vehicle such as physiological saline solution or citrate buffered saline.

### Sequences

Sequences of additional polypeptides described herein are as follows:

| SEQ | |
|---|---|
| ID NO | SEQUENCE |
| 1 | MPDLELNFAIPLHLEDDETVFTHDATNDNSQASSSYSSKSSPASANARKRISRKEMSGPPSKEPA |
| | NTKSRRANSQNNKLSLADRLTKYNIDEEFYQTRSDSLLSLNYTKKQIERLILYKGRISAVQQLLC |
| | KHEELLNLISPDGLGHKELIKIAARNGGGNNLIAVLSCYAKLKEMGFSSQQIIRMVSHAGGANNL |
| | KAVTANHDDLQNMGFNVEQIVRMVSHNGGSKNLKAVTDNHDDLKNMGFNAEQIVRMVSHGGGSKN |
| | LKAVTDNHDDLKNMGFNAEQIVSMVSNNGGSKNLKAVTDNHDDLKNMGFNAEQIVSMVSNGGGSL |
| | NLKAVKKYHDALKDRGENTEQIVRMVSHDGGSLNLKAVKKYHDALRERKENVEQIVSIVSHGGGS |
| | LNLKAVKKYHDVLKDREFNAEQIVRMVSHDGGSLNLKAVIDNHDDLKNMGFNAEQIVRMVSHKGG |
| | SKNLALVKEYFPVFSSFHFTADQIVALICQSKQCFRNLKKNHQQWKNKGLSAEQIVDLILQETPP |
| | KPNFNNTSSSTPSPSAPSFFQGPSTPIPTPVLDNSPAPIFSNPVCFFSSRSENNTEQYLQDSTLD |
| | LDSQLGDPTKNFNVNNFWSLFPFDDVGYHPHSNDVGYHLHSDEESPFFDE |
| 140 | MPKTKITTVSHGYDLDLMSSLPNGDPNQAKQGKIYLSGNGVYVVRDVAGIVHRGQLEFAINLEQL |
| | EQKINEPAFKAVILEKTSRAVGYTISNECENVELNALAKAGENNLDIDKLIFRRSSRGTVQTVLN |
| | SYNILLEKPYN |
| 141 | YDNKKIISIAASNCGTETINTIMSTDEVEESDFLYFVTTVSTPVASQNLSSASNININYSNRFMT |
| | ARKKTSDDNTDEVEEDQHRDKRRSNGR |
| 142 | YDNKKIISIAASNCGTETINTIMSTDEVEESDFLYFVTTVSTPVASQNLSSASNININYSNRF |
| 144 | MPKTNQPKNLEAKSTKNKISLPQDPQTLNELKIKGYPQDLAERLIKKGSSLAVKTVLKDHEQLVN |
| | FFTHLQIIRMAAQKGGAKNITTALNEYNSLINLG |
| 145 | YMLSQEQFLRLIDHHSGHLNLSILLDEQQWQAINDLCLQPHHFGRQNALEKFLQQGQRKYQNLQE |
| | LEQFLFQDSADPMLLQETENQHEAEKINDCMDFILRLISATEPLDLQIEIEGIGLFSPSMHFDAT |
| | QANFSTPAANEEKIDNSATEAGVNSRKRKIAAAHQKQPPRKKIATPLSATFISTLITLAQSDNPR |
| | LEMASAEALMLKAPQKLAMGITVRKKTKCEGIAIITVTDKTKLNGWLSSASESTYSSVEAQGTRT |
| | VNNTHAFFSTPLTSDKKSPSFSSLDFYEDSGLGFDEEIINPPYMPELEPEFIL |
| 146 | YMLSQEQFLRLIDHHSGHLNLSILLDEQQWQAINDLCLQPHHFGRQNALEKFLQQGQRKYQNL |
| 148 | MTLTLKQEKIAINKKLRSYRISKRKFLLDFSKMNLSPEGLNYAQEELAKLQFQAKASLETDQGIN |
| | IEEQLYQLGYTQSHLRPCADRYNCSILLNILLINNNSFVTQEISLENRVNLVVAANGNNDGIQVF |
| | FKTYPKLKSVG |
| 149 | MRLHALNNYHFLFHSIENIQLLITILQSHLEAFRIEQYMISGVLLNLLKQGQVISEQPCKILIDS |
| | SILNPNICQTLSNIINKYQFKNKPLYFNPTTSIITCMLSTQECYQLLAVWERRNISPSEILNNLL |
| | NPINIFQYQLISQTNEPDVYFLDCYHWHKFYPNMEIKQLQQLLIKAINLGINNCDILPEDNRTLI |
| | IEPYNDNWIKLSISIIDTIMDDSFNNLTRELFFCQLAPDSSNLIDDAIYIYKTQQTIEFLVISKS |
| | RSSERFILDTSTIYKDTIEEIEQALTHKLGALKGATYHTLIKCLLAQGYQVIGYFSMNIIGADVM |
| | PPTIIADDYPEYITLEWLSSEPMSQRSRLRTHDINSIKTLHNPTPKSQAIHQMLNLLALPDAISP |
| | LDSIQNNHISANHEQQTQGRISPISQQLDITLMRSRKRPLQKSDNTIYHDKRYWTFIGEGSYNKA |
| | YTDGQGFVVKVAKNELGLMDKSERSVRVFNEINPTLPQEVLAHVSQDLWISPLIENETLSPIEQA |
| | SFIFKTYIEHGRLILDGYCQNNLLQSAKYNTPVCIDPGNVVRRNSIASQEHWYAANEKTLLRRQL |
| | YRKHMIDTIDHYHKIRHIDRILPILMILALDFIDRKMQHLQLQLILKKNIKSLGIAFYFYYKHNQ |
| | SSTQQEFILSANIIDKILYGDQYICDTLDQSFKTLNKSRVVTLFRQINIDMSLI |
| 150 | MRLHALNNYHFLFHSIENIQLLITILQSHLEAFRIEQYMISGVLLNL |
| 159 | FTADQIVALICQSKQCFRNLKKNHQQWKNKGLSAEQIVDLILQETPPKPNENNTSSSTP |
| | SPSAPSFFQGPSTPIPTPVLDNSPAPIFSNPVCFFSSRSENNTEQYLQDSTLDLDSQLG |
| | DPTKNFNVNNFWSLFPFDDVGYHPHSNDVGYHLHSDEESPFFDF |

### EXAMPLES

These examples are provided for illustrative purposes only and not to limit the scope of the claims provided herein.

### Example 1

### Manufacture of a Modular Animal Pathogen-Nucleic Acid Binding Domain (MAP-NBD)

This example illustrates the manufacture of a modular animal pathogen-nucleic acid binding domain (MAP-NBD). The MAP-NBD is derived from L. quateirensis. The MAP-NBD comprises a plurality of repeat domains from L. quateirensis, such as any one of SEQ ID NO: 2-SEQ ID NO: 10 or SEQ ID NO: 89, wherein each repeat domain is selected to recognize and bind to a particular nucleotide or base pair, thereby providing a MAP-NBD that targets and binds a nucleic acid sequence of interest. The nucleic acid is DNA or RNA. The MAP-NBD is recombinantly expressed or synthetically constructed.

### Example 2

### Manufacture of a Modular Animal Pathogen-Nucleic Acid Binding Domain (MAP-NBD)-Nuclease

This example illustrates the manufacture of a modular animal pathogen-nucleic acid binding domain (MAP-NBD)-nuclease. The MAP-NBD is derived from L. quateirensis. The MAP-NBD comprises a plurality of repeat domains from L. quateirensis, such as any one of SEQ ID NO: 2-SEQ ID NO: 10, SEQ ID NO: 89, or SEQ ID NO: 33, where each repeat domain is selected to recognize and bind to a particular nucleotide or base pair, thereby providing a MAP-NBD that targets and binds a nucleic acid sequence of interest. The MAP-NBD-nuclease is recombinantly expressed or synthetically constructed. The MAP-NBD is linked to a nuclease via an optional linker. The linker is a synthetic linker, the full length C-terminus of the naturally occurring L. quateirensis protein set forth in SEQ ID NO: 1, or a fragment of the C-terminus of the naturally occurring L. quateirensis protein set forth in SEQ ID NO: 1. The nuclease is a cleavage domain (e.g., meganucleases such as I-AniI or I-OnuI) or a cleavage half domain (e.g., FokI nuclease, for example SEQ ID NO: 11, or Bfil).

### Example 3

### Manufacture of a Modular Animal Pathogen-Nucleic Acid Binding Domain (MAP-NBD)-Gene Regulator (MAP-NBD-GR)

This example illustrates the manufacture of a modular animal pathogen-nucleic acid binding domain (MAP-NBD)-gene regulator (MAP-NBD-GR). The MAP-NBD is derived from L. quateirensis. The MAP-NBD comprises a plurality of repeat domains from L. quateirensis, such as any one of SEQ ID NO: 2-SEQ ID NO: 10, SEQ ID NO: 89, or SEQ ID NO: 33, wherein each repeat domain is selected to recognize and bind to a particular nucleotide or base pair, thereby providing a MAP-NBD that targets and binds a nucleic acid sequence of interest. The MAP-NBD-GR is recombinantly expressed or synthetically constructed. The MAP-NBD is linked to a gene regulatory domain via an optional linker. The linker is a synthetic linker, the full length C-terminus of the naturally occurring L. quateirensis protein set forth in SEQ ID NO: 1, or a fragment of the C-terminus of the naturally occurring L. quateirensis protein set forth in SEQ ID NO: 1. The gene regulator is a gene activator (e.g., VP16, VP64, p65, p300 catalytic domain, TET1 catalytic domain, TDG, Ldb1 self-associated domain, SAM activator (VP64, p65, HSF1), or VPR (VP64, p65, Rta)) or a gene repressor (KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2).

### Example 4

### Manufacture of a Modular Animal Pathogen-Nucleic Acid Binding Domain (MAP-NBD)-Fluorophore

This example illustrates the manufacture of a modular animal pathogen-nucleic acid binding domain (MAP-NBD)-fluorophore. The MAP-NBD is derived from L. quateirensis. The MAP-NBD comprises a plurality of repeat domains from L. quateirensis, such as any one of SEQ ID NO: 2-SEQ ID NO: 10, SEQ ID NO: 89, or SEQ ID NO: 33, wherein each repeat domain is selected to recognize and bind to a particular nucleotide or base pair, thereby providing a MAP-NBD that targets and binds a nucleic acid sequence of interest. The MAP-NBD-fluorophore is recombinantly expressed or synthetically constructed. The MAP-NBD is linked to a fluorophore via an optional linker. The linker is a synthetic linker, the full-length C-terminus of the naturally occurring L. quateirensis protein set forth in SEQ ID NO: 1, or a fragment of the C-terminus of the naturally occurring L. quateirensis protein set forth in SEQ ID NO: 1. The fluorophore is a fluorescent moiety, such as green fluorescent protein or mCHERRY.

### Example 5

### Genome Editing with a Modular Animal Pathogen-Nucleic Acid Binding Domain (MAP-NBD)-Nuclease

This example illustrates genome editing with a modular animal pathogen-nucleic acid binding domain (MAP-NBD)-nuclease. The MAP-NBD-nuclease is manufactured as set forth in EXAMPLE 1 and EXAMPLE 2. The MAP-NBD is engineered to comprise a plurality of repeat sequences that will target a nucleic acid sequence of interest. When using a cleavage half-domain as the nuclease (e.g., FokI), two MAP-NBDs are engineered and administered, each with a FokI cleavage half-domain. A first MAP-NBD covalently linked to a FokI cleavage half domain is engineered to bind to first target nucleic acid sequence and a second MAP-NBD covalently linked to another FokI cleavage half domain is engineered to bind to a second target nucleic acid sequence, where the first and second target nucleic acid sequences are suitably spaced apart to provide for dimerization of the FokI cleavage half domains upon binding of the first and second MAP-NBDs to their respective target nucleic acid sequences. Upon administration to a subject, MAP-NBDs bind to their target nucleic acid sequences. Dimerization of two FokI cleavage half domains results in enzyme activity and inducement of a double stranded break (DSB). When using a cleavage domain (e.g., meganucleases such as I-AniI or I-OnuI or a fusion of two FokI domains), a single MAP-NBD bound to the cleavage domain induces a DSB in a target nucleic acid sequence after administration to a subject. The subject is a cell, a plurality of cells, or an animal, such as a human or non-human primate. The nuclease induces a double stranded break in the target sequence resulting in non-homologous end joining or homology directed repair, thereby providing genome editing functionality.

### Example 6

### Gene Regulation with a Modular Animal Pathogen-Nucleic Acid Binding Domain (MAP-NBD)-Gene Regulator (MAP-NBD-GR)

This example illustrates gene regulation with a modular animal pathogen-nucleic acid binding domain (MAP-NBD)-gene regulator (MAP-NBD-GR). The MAP-NBD-GR is manufactured as set forth in EXAMPLE 1 and EXAMPLE 3. The MAP-NBD is engineered to comprise a plurality of repeat sequences that will target a nucleic acid sequence of interest. The MAP-NBD-GR is administered to a subject. The subject is a cell, a plurality of cells, or an animal, such as a human or non-human primate. The MAP-NBD-GR then regulates gene expression (e.g., an activating or repressing domain), as set forth in EXAMPLE 3.

### Example 7

### Imaging with a Modular Animal Pathogen-Nucleic Acid Binding Domain (MAP-NBD)-Fluorophore

This example illustrates imaging a genomic locus in a cell with a modular animal pathogen-nucleic acid binding domain (MAP-NBD)-fluorophore. The MAP-NBD-fluorophore is manufactured as set forth in EXAMPLE 1 and 4. The MAP-NBD is engineered to comprise a plurality of repeat sequences that will target a nucleic acid sequence of interest. The MAP-NBD-fluorophore is administered to a cell. The cell is in a plurality of cells ex vivo, or is in an animal, such as a human or non-human primate. The MAP-NBD-fluorophore hybridizes to a target nucleic acid sequence, thereby labeling said target nucleic acid sequence. Imaging allows for visualization of labeled target cells, wherein labeled target cells indicate that the target genomic locus is present.

### Example 8

### Base Specificity of Repeats from L. Quateirensis

This example illustrates determining the base specificity of repeats from L. quateirensis. A polypeptide comprising a L. quateirensis protein of sequence set forth in SEQ ID NO: 1 fused to a purification tag was constructed. Base specificity of each of the repeats of SEQ ID NO: 89 and SEQ ID NO: 2-SEQ ID NO: 10 was determined by SELEX.

In vitro transcription and translation (IVTT) of target polypeptides was carried out using the Thermo Fisher 1-Step Human Coupled IVT Kit. The following reagents were added into a nuclease-free tube and mixed: Hela lysate (15 μl), accessory proteins (3 μl), reaction mix (6 μl), plasmid DNA (0.2-0.5 μg/μl) (2.4 μl), and nuclease-free H₂O (3.6 μl). Reactions are carried for 120 minutes to 6 hours at 30° C.

Protein expression was detected by Western blot using appropriate primary antibodies and HRP-conjugated secondary antibodies.

Oligonucleotide target libraries were chemically synthesized at random with a mixture of all 4 phophoramidites. Each oligonucleotide sequence is flanked by discrete 20 base pair sequences as follows: ACACGACGCTCTTCCGATCTNNNNNNNNNNNNNNNNNNNNNNNNNAGATCGGAAGAG CACACGTC, where N is any one of the 4 phophoramidites. Oligonucleotide sequences in the library were converted to double-stranded duplexes by annealing each sequence with the 3′ library primer (5′-GACGTGTGCTCTTCCGATCT) and incubating at following conditions:

TABLE 4
Conditions for Generation of Library of Double-Stranded Duplexes

| Component | Stock | Final | Amount (100 μl) |
|---|---|---|---|
| N-mer oligo target library | 250 μM | 20 μM | 8 μl |
| Primer | 500 μM | 60 μM | 12 μl |
| Thermo Pol buffer | 10 x | 1 x | 10 μl |
| dNTP | 100 mM each | 1.25 mM each | 1.25 μl each |
| MgSO4 | 100 mM | 3 mM | 3 μl |
| Vent (exo-) | | | 3 μl |
| ddH2O | | | 59 μl |

95° C. 3 min −> 62° C. 10 hour −> 4° C.

The oligonucleotide library was digested with ExoI to remove ssDNA at 37° C. for 2.5 hours as follows.

TABLE 5
Digestion Conditions

| Component | Amount (120 μl) |
|---|---|
| Primer extension reaction | 100 μl |
| Exo I buffer | 12 μl |
| Exo I | 4 μl |
| ddH2O | 4 μl |

DNA was cleaned up with phenol: chloroform: iso-amyl alcohol and EtOH precipitation (−70° C. for 1.5 hours). Duplex libraries were resuspended in QIAGEN EB buffer.

SELEX was carried out using the following SELEX buffers.

TABLE 6
SELEX buffer (for binding)

| Component | Stock | Final | Amount (10 ml) |
|---|---|---|---|
| Tween 20 | 10% | 0.1% | 0.1 ml |
| ZnCl2 | 0.1M | 10 μM | 1 μl |
| MgCl2 | 1M | 0.5 mM | 5 μl |
| poly dIdC | 2 μg/μl | 0.02 μg/μl | 0.1 ml |
| BSA | 0.5% | 0.01% | 0.2 ml |
| DPBS, Ca2+ free | | 1 x | 9.394 ml |
| Proteinase inhibitor cocktail | 50 x | 1 x | 0.2 ml |

TABLE 7
SELEX wash buffer I (same as SELEX
binding buffer, but without poly dIdC)

| | | | Amount |
|---|---|---|---|
| Component | Stock | Final | (50 ml) |
| Tween 20 | 10% | 0.1% | 0.5 ml |
| ZnCl2 | 0.1 M | 10 μM | 5 μl |
| MgCl2 | 1 M | 0.5 mM | 0.025 ml |
| BSA | 0.5% | 0.01% | 1 ml |
| DPBS, Ca2 + free | | 1 x | 47.47 ml |
| Proteinase inhibitor | 50 x | 1 x | 1 ml |
| cocktail | | | |

*In DPBS, [NaCl] = 137 mM

TABLE 8
SELEX wash buffer II (without poly
dIdC, with additional 75 mM NaCl)

| | | | Amount |
|---|---|---|---|
| Component | Stock | Final | (50 ml) |
| Tween 20 | 10% | 0.1% | 0.5 ml |
| DTT | 1 M | 5 mM | 0.25 ml |
| ZnCl2 | 0.1 M | 10 μM | 5 μl |
| MgCl2 | 1 M | 5 mM | 0.025 ml |
| NaCl | 5 M | 75 mM | 0.75 ml |
| BSA | 0.5% | 0.01% | 1 ml |
| DPBS, Ca2 + free | | 1 x | 46.47 ml |
| Proteinase inhibitor | 50 x | 1 x | 1 ml |
| cocktail | | | |

*In DPBS, [NaCl] = 137 mM

For each polypeptide tested, 25 μl Dynabeads®His-Tag or Pierce™ anti-HA magnetic beads were washed once with 200 μl SELEX binding buffer. Beads were resuspended in 25 μl SELEX binding buffer and left on ice.

SELEX Cycle 1. Bead-protein complexes were mixed with 200 pmol of the oligonucleotide duplex library in a total volume of 100 μl SELEX buffer. The reaction product of the in vitro transcription and translation reaction was pre-incubated with SELEX buffer for 10 min and then added to the library duplex.

TABLE 9
Ratios of IVTT protein, SELEX
buffer and Library duplex

| | | | Amount |
|---|---|---|---|
| Component | Stock | Final | (100 μl) |
| IVTT protein | | | 8 μl |
| SELEX buffer | | | 82 μl |
| Library duplex | 40 μM | 4 μM | 10 μl |

Tubes were placed at a low angle on the rotator and incubated at room temperature for 50 minutes. 8 μl of washed beads (in SELEX binding buffer) was added to the complexes and incubated on the rotator for 20 minutes. Tubes were placed on a magnet for 2 min and the supernatant was discarded. Beads were washed 6 times with the SELEX wash buffer. The bound DNA target (with beads) were PCR amplified using the 5′/3′ library primers: fwd: ACACGACGCTCTTCCGATCT rev: GACGTGTGCTCTTCCGATCT

TABLE 10
PCR Parameters

| | | | Amount |
|---|---|---|---|
| Component | Stock | Final | (50 μl) |
| Jumpstart buffer | 10 x | 1 x | 5 μl |
| 10 mM dNTP | | | 1 μl |
| Forward primer | 10 μM | 0.5 μM | 2.5 μl |
| Reverse primer | 10 μM | 0.5 μM | 2.5 μl |
| Jump Start Taq DNA Pol | | | 1 μl |
| Template | | | Bead bound DNA |
| PCR-grade H2O | | | 38 μL |

95° C. 2 min -> 25 x [95° C. 10 s -> 64° C. 20 s -> 72° C. 15 s] -> 72° C. 3 min

PCR-1 reaction was separated from the beads on the magnet. 5 μl of PCR-1 reaction was used for PCR-2 amplification and μl PCR-2 in SELEX Cycle 2.

TABLE 11
PCR Parameters

| | | | Amount |
|---|---|---|---|
| Component | Stock | Final | (30 μl) |
| Jumpstart buffer | 10 x | 1 x | 3 μl |
| 10 mM dNTP | | | 0.6 μl |
| Forward primer | 10 μM | 0.5 μM | 1.5 μl |
| Reverse primer | 10 μM | 0.5 μM | 1.5 μl |
| Jump Start Taq DNA Pol | | | 0.6 μl |
| PCR-1from 1st SELEX cycle | | | 5 μl |
| PCR-grade H2O | | | 17.8 μl |

95° C. 2 min -> 3 x [95° C. 10 s -> 64° C. 20 s -> 72° C. 15 s] -> 72° C. 3 min

SELEX Cycle 2. Bead-protein complex were mixed with the PCR product from the 1^{st} cycle in a total volume of 100 μl SELEX buffer. The in vitro transcription and translation (IVTT) reaction was pre-incubated with SELEX buffer for 10 min.

TABLE 12
Ratios of IVTT protein, SELEX
buffer and Library duplex

| Component | Stock | Final | Amount (100 μl) |
|---|---|---|---|
| IVTT protein | | | 8 μl |
| SELEX buffer | | | 67 μl |
| PCR-2 from 1^{st} SELEX | | | 25 μl |

Tubes were placed at a low angle on the rotator and incubate at room temperature for 50 minutes. 8 μl of washed beads (in SELEX binding buffer) was added to the complexes and incubated on the rotator for 20 minutes. Tubes were placed on a magnet for 2 min and the supernatant was discarded. Beads were washed 6 times with SELEX was buffer. The bound DNA target (with beads) was PCR amplified using the 5′/3′ library primers: fwd: ACACGACGCTCTTCCGATCT and rev:

GACGTGTGCTCTTCCGATCT.

TABLE 13
PCR Parameters

| | | | Amount |
|---|---|---|---|
| Component | Stock | Final | (50 μl) |
| Jumpstart buffer | 10 x | 1 x | 5 μl |
| 10 mM dNTP | | | 1 μl |
| Forward primer | 10 μM | 0.5 μM | 2.5 μl |
| Reverse primer | 10 μM | 0.5 μM | 2.5 μl |
| Jump Start Taq DNA Pol | | | 1 μl |
| Template | | | Bead bound DNA |
| PCR-grade H2O | | | 38 μl |

95° C. 2 min -> 25 x [95° C. 10 s -> 64° C. 20 s -> 72° C. 15 s] -> 72° C. 3 min

PCR-1 reaction was separated from the beads on the magnet. 5 μl of PCR-1 reaction was used for PCR-2 amplification and 25 μl of PCR-2 in the 3^{rd} SELEX cycle.

TABLE 14
PCR Parameters

| | | | Amount |
|---|---|---|---|
| Component | Stock | Final | (30 μl) |
| Jumpstart buffer | 10 x | 1 x | 3 μl |
| 10 mM dNTP | | | 0.6 μl |
| Forward primer | 10 μM | 0.5 μM | 1.5 μl |
| Reverse primer | 10 μM | 0.5 μM | 1.5 μl |
| Jump Start Taq DNA Pol | | | 0.6 μl |
| PCR-1from 2^{nd} SELEX cycle | | | 5 μl |
| PCR-grade H2O | | | 17.8 μl |

95° C. 2 min -> 3 x [95° C. 10 s -> 64° C. 20 s -> 72° C. 15 s] ->72° C. 3 min

SELEX Cycle 3. Mix IVTT protein with PCR product from 1st cycle in a total volume of 100 μl SELEX buffer. The in vitro transcription and translation (IVTT) reaction was pre-incubated with SELEX buffer for 10 min.

TABLE 15
Ratios of IVTT protein, SELEX
buffer and Library duplex

| | | | Amount |
|---|---|---|---|
| Component | Stock | Final | (100 μl) |
| IVTT protein | | | 8 μl |
| SELEX buffer | | | 67 μl |
| PCR-2 from 2^{nd} SELEX | | | 25 μl |

Tubes were placed at a low angle on the rotator and incubate at room temperature for 50 minutes. 8 μl of washed beads (in SELEX binding buffer) were added to the complexes and incubated on the rotator for 20 minutes. Tubes were placed on a magnet for 2 min and the supernatant was discarded. Beads were washed 6 times with the SELEX wash buffer. The bound DNA target (with the beads) were PCR amplified using the 5′/3′ library primers: fwd: ACACGACGCTCTTCCGATCT, rev:

GACGTGTGCTCTTCCGATCT.

TABLE 16
PCR Parameters

| | | | Amount |
|---|---|---|---|
| Component | Stock | Final | (50 μl) |
| Jumpstart buffer | 10 x | 1 x | 5 μl |
| 10 mM dNTP | | | 1 μl |
| Forward primer | 10 μM | 0.5 μM | 2.5 μl |
| Reverse primer | 10 μM | 0.5 μM | 2.5 μl |
| Jump Start Taq DNA Pol | | | 1 μl |
| Template | | | Bead bound DNA |
| PCR-grade H2O | | | 38 μl |

95° C. 2 min -> 25 x [95° C. 10 s -> 64° C. 20 s -> 72° C. 15 s] -> 72° C. 3 min

The PCR-1 reaction was separated from the beads on the magnet. 5 μl of PCR-1 reaction was used for PCR-2 amplification.

TABLE 17
PCR Parameters

| | | | Amount |
|---|---|---|---|
| Component | Stock | Final | (30 μl) |
| Jumpstart buffer | 10 x | 1 x | 3 μl |
| 10 mM dNTP | | | 0.6 μl |
| Forward primer | 10 μM | 0.5 μM | 1.5 μl |
| Reverse primer | 10 μM | 0.5 μM | 1.5 μl |
| Jump Start Taq DNA Pol | | | 0.6 μl |
| PCR-1from 3^{rd} SELEX cycle | | | 5 μl |
| PCR-grade H2O | | | 17.8 μl |

95° C. 2 min -> 3 x [95° C. 10 s -> 64°C. 20 s -> 72° C. 15 s] -> 72° C. 3 min

PCR-1 reactions from the 3^{rd} SELEX cycle were separated from the beads on the magnet. PCR-1 reactions from the 1^{st}, 2^{nd}, and 3^{rd} SELEX cycles were analyzed by MiniSeq.

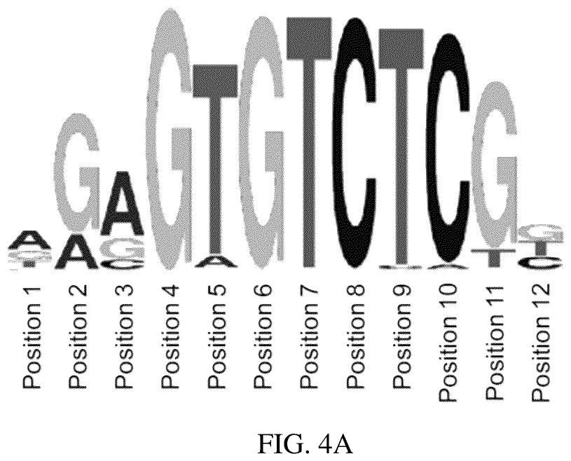

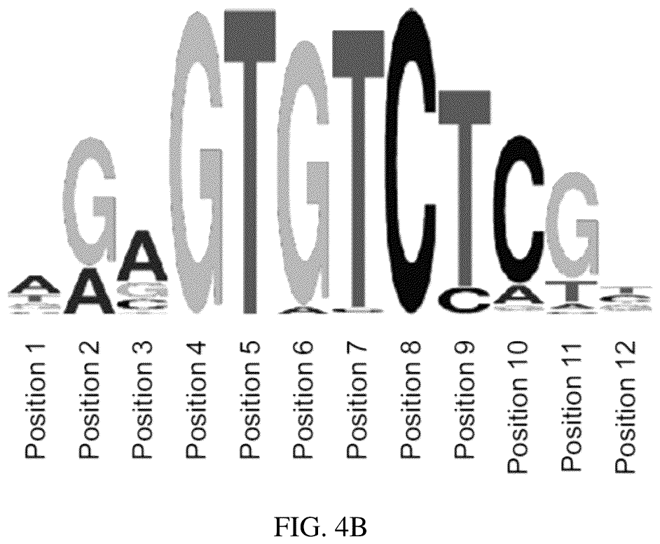

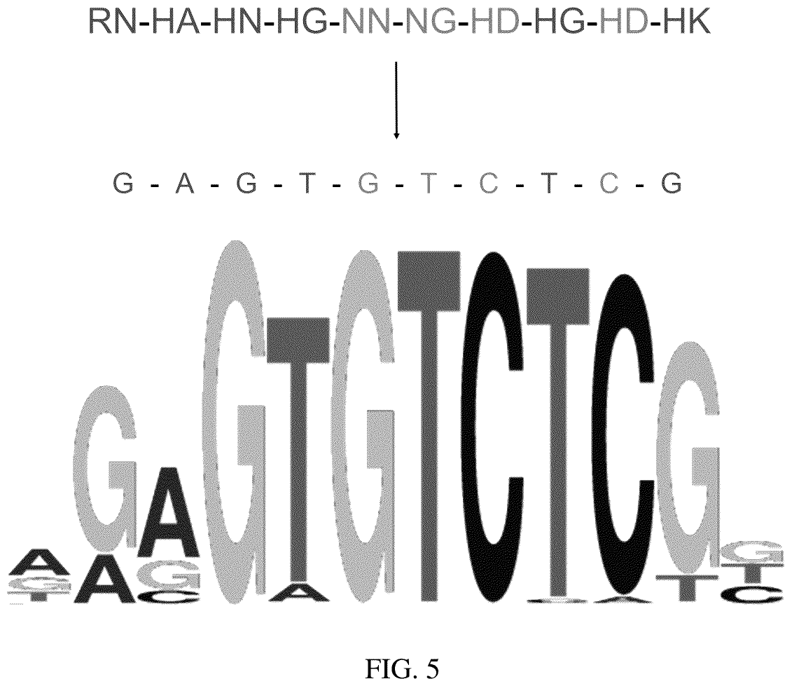

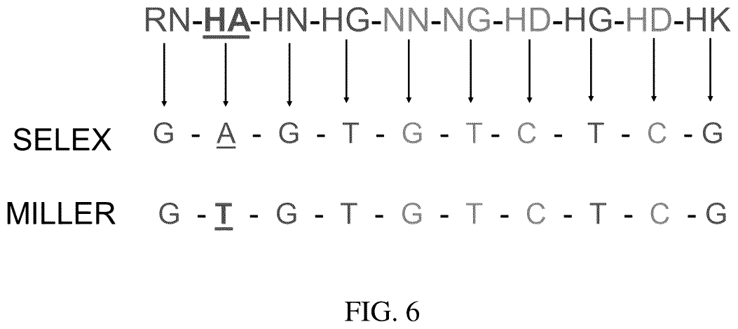

Paired-end reads were merged with BBmerge. A 25-base pair variable-region sequence was extracted from between recognized constant regions. 5000 sequences were drawn at random from the full set, including duplicates, and given as an input to the GADEM motif discovery program with parameters: ‘-maskR 1-fullScan 1-gen 3’. Sequence logos in FIGS. 4A and 4B were drawn using the ‘ceqlogo’ tool in the MEME suite, from the GADEM MEME-format motif files.

FIGS. 4A and 4B illustrate the binding motifs determined for a polypeptide of SEQ ID NO:1 and comprising the sequence LGHKELIKIAARNGGGNNLIAVLSCYAKLKEMG (SEQ ID NO: 89) present in an N-terminus of L. quateirensis followed by each of the repeats of SEQ ID NO: 2-SEQ ID NO: 10. The larger the size the of the base at a particular position, the higher the relative frequency at which the base is present at that position in a nucleic acid bound by the tested polypeptide.

FIG. 4A illustrates the bases to which the repeats (SEQ ID NOs: 89 and 2-10 ordered from N-terminus to C-terminus) in a polypeptide having the amino acid sequence set forth in SEQ ID NO: 1 bind. The polypeptide having the amino acid sequence set forth in SEQ ID NO:1 was fused to a His-tag.

FIG. 4B illustrates the bases to which the repeats (from N-terminus to C-terminus: SEQ ID NOs: 89 and 2-10) in a polypeptide having the amino acid sequence set forth in SEQ ID NO:1 bind. The polypeptide having the amino acid sequence set forth in SEQ ID NO:1 was fused to a HA-tag. Both the His-tagged polypeptide and the HA-tagged polypeptide showed the same binding specificity.

A numerical representation of position weight matrices (PWMs) of FIG. 4A and FIG. 4B is shown in the tables below. Background letter frequencies from a uniform background were as follows: A—0.25000; C—0.25000; G—0.25000; and T—0.25000.

TABLE 18
MEME Data Expressing the Position Weight Matrix (PWM) for His-Tagged
Protein after 3 Rounds of SELEX

| Binding Motif: rGAGTGTCTCGb | A | C | G | T |
|---|---|---|---|---|
| Position 1 of FIG. 4A | 0.493 | 0.049 | 0.260 | 0.198 |
| Position 2 of FIG. 4A corresponding to the base | 0.229 | 0.000 | 0.769 | 0.002 |
| specificity of a repeat from L. quateirensis of | | | | |
| SEQ ID NO: 96 | | | | |
| Position 3 of FIG. 4A corresponding to the base | 0.665 | 0.108 | 0.224 | 0.003 |
| specificity of a repeat from L. quateirensis of | | | | |
| SEQ ID NO: 2 | | | | |
| Position 4 of FIG. 4A corresponding to the base | 0.000 | 0.000 | 1.000 | 0.000 |
| specificity of a repeat from L. quateirensis of | | | | |
| SEQ ID NO: 3 | | | | |
| Position 5 of FIG. 4A corresponding to the base | 0.069 | 0.003 | 0.004 | 0.924 |
| specificity of a repeat from L. quateirensis of | | | | |
| SEQ ID NO: 4 | | | | |
| Position 6 of FIG. 4A corresponding to the base | 0.006 | 0.000 | 0.994 | 0.000 |
| specificity of a repeat from L. quateirensis of | | | | |
| SEQ ID NO: 5 | | | | |
| Position 7 of FIG. 4A corresponding to the base | 0.001 | 0.006 | 0.000 | 0.992 |
| specificity of a repeat from L. quateirensis of | | | | |
| SEQ ID NO: 6 | | | | |
| Position 8 of FIG. 4A corresponding to the base | 0.001 | 0.992 | 0.006 | 0.000 |
| specificity of a repeat from L. quateirensis of | | | | |
| SEQ ID NO: 7 | | | | |
| Position 9 of FIG. 4A corresponding to the base | 0.003 | 0.018 | 0.000 | 0.979 |
| specificity of a repeat from L. quateirensis of | | | | |
| SEQ ID NO: 8 | | | | |
| Position 10 of FIG. 4A corresponding to the | 0.025 | 0.973 | 0.001 | 0.001 |
| base specificity of a repeat from L. quateirensis | | | | |
| of SEQ ID NO: 9 | | | | |
| Position 11 of FIG. 4A corresponding to the | 0.007 | 0.000 | 0.886 | 0.107 |
| base specificity of a repeat from L. quateirensis | | | | |
| of SEQ ID NO: 10 | | | | |
| Position 12 of FIG. 4A | 0.009 | 0.293 | 0.370 | 0.328 |

TABLE 19
MEME Data Expressing the Position Weight Matrix (PWM) for HA-Tagged
Protein after 3 Rounds of SELEX

| Binding Motif: wGAGTGTCTCGv | A | C | G | T |
|---|---|---|---|---|
| Position 1 of FIG. 4B | 0.492 | 0.067 | 0.200 | 0.240 |
| Position 2 of FIG. 4B corresponding to the base | 0.280 | 0.000 | 0.708 | 0.012 |
| specificity of a repeat from L. quateirensis of | | | | |
| SEQ ID NO: 96 | | | | |
| Position 3 of FIG. 4B corresponding to the base | 0.655 | 0.130 | 0.186 | 0.030 |
| specificity of a repeat from L. quateirensis of | | | | |
| SEQ ID NO: 2 | | | | |
| Position 4 of FIG. 4B corresponding to the base | 0.005 | 0.000 | 0.998 | 0.000 |
| specificity of a repeat from L. quateirensis of | | | | |
| SEQ ID NO: 3 | | | | |
| Position 5 of FIG. 4B corresponding to the base | 0.107 | 0.013 | 0.015 | 0.865 |
| specificity of a repeat from L. quateirensis of | | | | |
| SEQ ID NO: 4 | | | | |
| Position 6 of FIG. 4B corresponding to the base | 0.041 | 0.002 | 0.956 | 0.000 |
| specificity of a repeat from L. quateirensis of | | | | |
| SEQ ID NO: 5 | | | | |
| Position 7 of FIG. 4B corresponding to the base | 0.023 | 0.021 | 0.001 | 0.956 |
| specificity of a repeat from L. quateirensis of | | | | |
| SEQ ID NO: 6 | | | | |
| Position 8 of FIG. 4B corresponding to the base | 0.028 | 0.968 | 0.003 | 0.000 |
| specificity of a repeat from L. quateirensis of | | | | |
| SEQ ID NO: 7 | | | | |
| Position 9 of FIG. 4B corresponding to the base | 0.009 | 0.082 | 0.001 | 0.908 |
| specificity of a repeat from L. quateirensis of | | | | |
| SEQ ID NO: 8 | | | | |
| Position 10 of FIG. 4B corresponding to the | 0.093 | 0.898 | 0.026 | 0.002 |
| base specificity of a repeat from L. quateirensis | | | | |
| of SEQ ID NO: 9 | | | | |
| Position 11 of FIG. 4B corresponding to the | 0.032 | 0.008 | 0.802 | 0.158 |
| base specificity of a repeat from L. quateirensis | | | | |
| of SEQ ID NO: 10 | | | | |
| Position 12 of FIG. 4B | 0.034 | 0.325 | 0.296 | 0.345 |

### Example 9

### Manufacture of a Modular Animal Pathogen-Nucleic Acid Binding Domain (MAP-NBD)

This example illustrates the manufacture of a modular animal pathogen-nucleic acid binding domain (MAP-NBD). The MAP-NBD is derived from L. quateirensis, Burkholderia, Paraburkholderia, Francisella, or any combination thereof. The MAP-NBD comprises a plurality of repeat domains from L. quateirensis, Burkholderia, Paraburkholderia, Francisella, or any combination thereof, such as any one of the repeats in Table 1, wherein each repeat domain is selected to recognize and bind to a particular nucleotide or base pair, thereby providing a MAP-NBD that targets and binds a nucleic acid sequence of interest. The nucleic acid is DNA or RNA. The MAP-NBD is recombinantly expressed or synthetically constructed.

### Example 10

### Manufacture of a Modular Animal Pathogen-Nucleic Acid Binding Domain (MAP-NBD)-Nuclease

This example illustrates the manufacture of a modular animal pathogen-nucleic acid binding domain (MAP-NBD)-nuclease. The MAP-NBD is derived from L. quateirensis, Burkholderia, Paraburkholderia, Francisella, or any combination thereof. The MAP-NBD comprises a plurality of repeat domains from L. quateirensis, Burkholderia, Paraburkholderia, Francisella, or any combination thereof, such as any one of SEQ ID NO: 2-SEQ ID NO: 95, wherein each repeat domain is selected to recognize and bind to a particular nucleotide or base pair, thereby providing a MAP-NBD that targets and binds a nucleic acid sequence of interest. The MAP-NBD-nuclease is recombinantly expressed or synthetically constructed. The MAP-NBD is linked to a nuclease via an optional linker. The linker is a synthetic linker, the full-length C-terminus or a truncation thereof of the naturally occurring L. quateirensis, Burkholderia, Paraburkholderia, or Francisella protein. The nuclease is a cleavage domain (e.g., meganucleases such as I-AniI or I-OnuI) or a cleavage half domain (e.g., FokI nuclease, for example SEQ ID NO: 11, or Bfil).

### Example 11

### Manufacture of a Modular Animal Pathogen-Nucleic Acid Binding Domain (MAP-NBD)-Gene Regulator (MAP-NBD-GR)

This example illustrates the manufacture of a modular animal pathogen-nucleic acid binding domain (MAP-NBD)-gene regulator (MAP-NBD-GR). The MAP-NBD is derived from L. quateirensis, Burkholderia, Paraburkholderia, Francisella, or any combination thereof. The MAP-NBD comprises a plurality of repeat domains from L. quateirensis, Burkholderia, Paraburkholderia, Francisella, or any combination thereof, such as any one of the repeats listed in Table 1, wherein each repeat domain is selected to recognize and bind to a particular nucleotide or base pair, thereby providing a MAP-NBD that targets and binds a nucleic acid sequence of interest. The MAP-NBD-GR is recombinantly expressed or synthetically constructed. The MAP-NBD is linked to a gene regulatory domain via an optional linker. The linker is a synthetic linker, the full-length C-terminus or a truncation of the C-terminus of the naturally occurring L. quateirensis, Burkholderia, Paraburkholderia, or Francisella protein. The gene regulator is a gene activator (e.g., VP16, VP64, p65, p300 catalytic domain, TET1 catalytic domain, TDG, Ldb1 self-associated domain, SAM activator (VP64, p65, HSF1), or VPR (VP64, p65, Rta)) or a gene repressor (KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2).

### Example 12

### Manufacture of a Modular Animal Pathogen-Nucleic Acid Binding Domain (MAP-NBD)-Fluorophore

This example illustrates the manufacture of a modular animal pathogen-nucleic acid binding domain (MAP-NBD)-fluorophore. The MAP-NBD is derived from L. quateirensis, Burkholderia, Paraburkholderia, Francisella, or any combination thereof. The MAP-NBD comprises a plurality of repeat domains from L. quateirensis, Burkholderia, Paraburkholderia, Francisella, or any combination thereof, such as any one of the repeats listed in Table 1, wherein each repeat domain is selected to recognize and bind to a particular nucleotide or base pair, thereby providing a MAP-NBD that targets and binds a nucleic acid sequence of interest. The MAP-NBD-fluorophore is recombinantly expressed or synthetically constructed. The MAP-NBD is linked to a fluorophore via an optional linker. The linker is a synthetic linker, the full-length C-terminus or a truncation of the C-terminus of the naturally occurring L. quateirensis, Burkholderia, Paraburkholderia, or Francisella protein. The fluorophore is a fluorescent moiety, such as green fluorescent protein or mCHERRY.

### Example 13

### Genome Editing with a Modular Animal Pathogen-Nucleic Acid Binding Domain (MAP-NBD)-Nuclease

This example illustrates genome editing with a modular animal pathogen-nucleic acid binding domain (MAP-NBD)-nuclease. The MAP-NBD-nuclease is manufactured as set forth in EXAMPLE 9 and EXAMPLE 10. The MAP-NBD is engineered to comprise a plurality of repeat sequences that will target a nucleic acid sequence of interest. When using a cleavage half-domain as the nuclease (e.g., FokI), two MAP-NBDs are engineered and administered, each with a FokI cleavage half-domain. A first MAP-NBD covalently linked to a FokI cleavage half domain is engineered to bind to first target nucleic acid sequence and a second MAP-NBD covalently linked to another FokI cleavage half domain is engineered to bind to a second target nucleic acid sequence, where the first and second target nucleic acid sequences are suitably spaced apart to provide for dimerization of the FokI cleavage half domains upon binding of the first and second MAP-NBDs to their respective target nucleic acid sequences. Upon administration to a subject, MAP-NBDs bind to their target nucleic acid sequences. Dimerization of two FokI cleavage half domains results in enzyme activity and inducement of a double stranded break (DSB). When using a cleavage domain (e.g., meganucleases such as I-AniI or I-OnuI or a fusion of two FokI domains), a single MAP-NBD bound to the cleavage domain induces a DSB in a target nucleic acid sequence after administration to a subject. The subject is a cell, a plurality of cells, or an animal, such as a human or non-human primate. The nuclease induces a double stranded break in the target sequence resulting in non-homologous end joining or homology directed repair, thereby providing genome editing functionality.

### Example 14

### Gene Regulation with a Modular Animal Pathogen-Nucleic Acid Binding Domain (MAP-NBD)-Gene Regulator (MAP-NBD-GR)

This example illustrates gene regulation with a modular animal pathogen-nucleic acid binding domain (MAP-NBD)-gene regulator (MAP-NBD-GR). The MAP-NBD-GR is manufactured as set forth in EXAMPLE 9 and EXAMPLE 11. The MAP-NBD is engineered to comprise a plurality of repeat sequences that will target a nucleic acid sequence of interest. The MAP-NBD-GR is administered to a subject. The subject is a cell, a plurality of cells, or an animal, such as a human or non-human primate. The MAP-NBD-GR then regulates gene expression (ie via its activating or repressing domain).

### Example 15

### Imaging with a Modular Animal Pathogen-Nucleic Acid Binding Domain (MAP-NBD)-Fluorophore

This example illustrates imaging a genomic locus in a cell with a modular animal pathogen-nucleic acid binding domain (MAP-NBD)-fluorophore. The MAP-NBD-fluorophore is manufactured as set forth in EXAMPLE 9 and 12. The MAP-NBD is engineered to comprise a plurality of repeat sequences that will target a nucleic acid sequence of interest. The MAP-NBD-fluorophore is administered to a cell. The cell is in a plurality of cells ex vivo, or is in an animal, such as a human or non-human primate. The MAP-NBD-fluorophore hybridizes to a target nucleic acid sequence, thereby labeling said target nucleic acid sequence. Imaging allows for visualization of labeled target cells, wherein labeled target cells indicate that the target genomic locus is present.

### Example 16

### In Vitro Base Specificity of Repeats from L. Maceachernii

In order to determine the base specificity of repeats identified in the hypothetical protein WP_058451450 from L. maceachernii “LEGm,” DNA constructs to encode the full-length protein was engineered and produced. The full-length protein (having the sequence set forth in SEQ ID NO: 143) was synthesized by in vitro transcription and translation reactions and characterized using the SELEX-seq method. The identified sequence motifs were compared with the module structure of each protein to assign a DNA base preference to each repeat as well as the N-terminus and C-terminus. The base specificity of repeats identified in the hypothetical protein WP_WP_058473422 (SEQ ID NO:1) from L. quateirensis “LEGq” and described in Example 8, was further confirmed.

### Constructs

The LEGq and LEGm proteins were divided into modules based on their domain structure: N terminus, individual repeat units, and C terminus. DNA modules encoding these modules were engineered with flanking restriction sites and synthesized (Integrated DNA Technologies, Coralville, Iowa). These DNA modules were ligated together using a modified protocol from Reyon et al. 2013, (Current Protocols in Molecular Biology, 12.16.1) to produce a DNA construct encoding the full-length protein. In the case of the LEGq protein, a DNA construct encoding the entire full-length protein was synthesized. The LEGm construct was engineered to encode an N-terminal FLAG tag and a C-terminal HA tag to enable protein retention during SELEX, and the fully-assembled construct was cloned into the pVax_NG_63aa, propagated in E. coli and purified using standard methods. The LEGq construct was cloned into the pT7CFE1-CHA vector (Thermo Scientific), which added C-terminal HA tag to the protein, propagated in E. coli and purified using standard methods. Purified plasmid constructs were subjected to Sanger sequencing to verify the expected DNA sequence. The SEQ ID NOs for the individual modules are below:

| | L. quateirensis | SEQ | | L. maceachernii | SEQ |
|---|---|---|---|---|---|
| Motif | Protein | ID | Motif | Protein | ID |
| position | modules | NO | position | modules | NO |
| | LEGq | 13 | 1-2 | LEGm | 144 |
| | N-terminus | | | N-terminus | |
| 1 | LEG.RN.001 | 89 | 3 | LEG.HG.003 | 28 |
| 2 | LEG.HA.001 | 2 | 4 | LEG.HD.005 | 26 |
| 3 | LEG.HN.001 | 3 | 5 | LEG.H1.003 | 32 |
| 4 | LEG.HG.002 | 4 | 6 | LEG.HK.003 | 34 |
| 5 | LEG.NN.001 | 5 | 7 | LEG.H1.001 | 30 |
| 6 | LEG.NG.001 | 6 | 8 | LEG.H1.002 | 31 |
| 7 | LEG.HD.006 | 7 | 9 | LEG.HD.002 | 23 |
| 8 | LEG.HG.004 | 8 | 10 | LEG.HG.005 | 29 |
| 9 | LEG.HD.001 | 9 | 11 | LEG.HD.003 | 24 |
| 10 | LEG.HK.001 | 33 | 12 | LEG.HG.001 | 27 |
| | LEGq C- | 159 | 13 | LEG.HV.001 | 35 |
| | terminus | | 14 | LEG.H1.004 | 133 |
| | | | 15-17 | LEGm C- | 159 |
| | | | | terminus | |

### SELEX-Seq

In vitro transcription/translation (IVTT) kits were used to produce full-length protein from our plasmid constructs for LEGq (1-Step human Coupled IVT, Promega) and LEGm (TnT Quick Coupled Transcription/Translation kit, Thermo Scientific) using the manufacturer's protocols. The SELEX-seq protocol was adapted from Miller et al. 2011 (supra). Briefly, a library of random sequences 25 nucleotides (nt) in length, flanked by common 20-nt adapter sequences and ordered as single-stranded DNA oligonucleotides was created. A double-stranded DNA library was created using primer extension.

The IVTT proteins were subjected to three SELEX cycles of: allowing proteins to bind to double-stranded DNA; immobilization of protein: DNA complexes on anti-HA magnetic beads and washing off unbound DNA; PCR amplification of bound DNA. In this way, the DNA molecules bound by the proteins were used as input for successive rounds of binding to the LEGq and LEGq proteins, enriching the preferred binding sequence of these proteins within the pool of DNA molecules at each cycle.

PCR samples from each SELEX cycle were diluted 1:50 and amplified using indexed primers recognizing the common adapters flanking the 25-nt random sequences. PCRs were pooled, purified, and sequenced on an Illumina MiniSeq instrument.

### SELEX Seq Data Analysis

Sequence reads from the MiniSeq were de-multiplexed using the index sequences of the primers to assign sequence reads to individual SELEX samples. These reads were then filtered to require constant sequences matching the common adapters flanking a variable region of 25 nucleotides. A random sample of 5,000 reads from each sample was used as input to the GADEM motif finder (Li et al. 2009, Journal of Computational Biology, 16(2):317-29). Discovered motifs were visualized using the “ceqlogo” function of the MEME suite (Bailey et al. 2015, Nucleic Acids Research, 43(W1):W39-49).

### Results

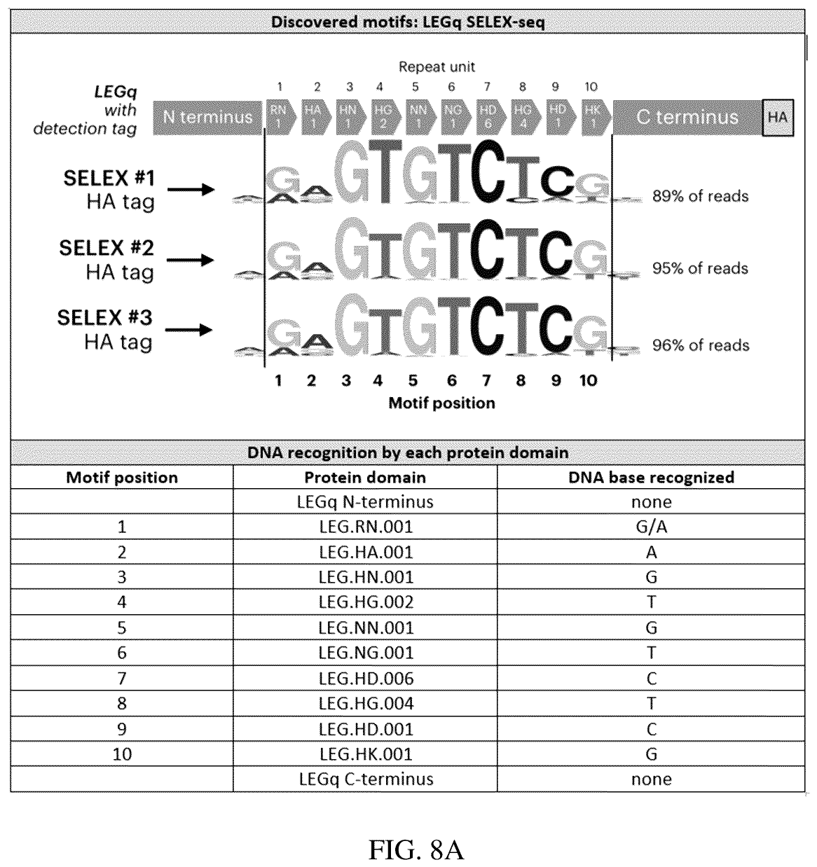

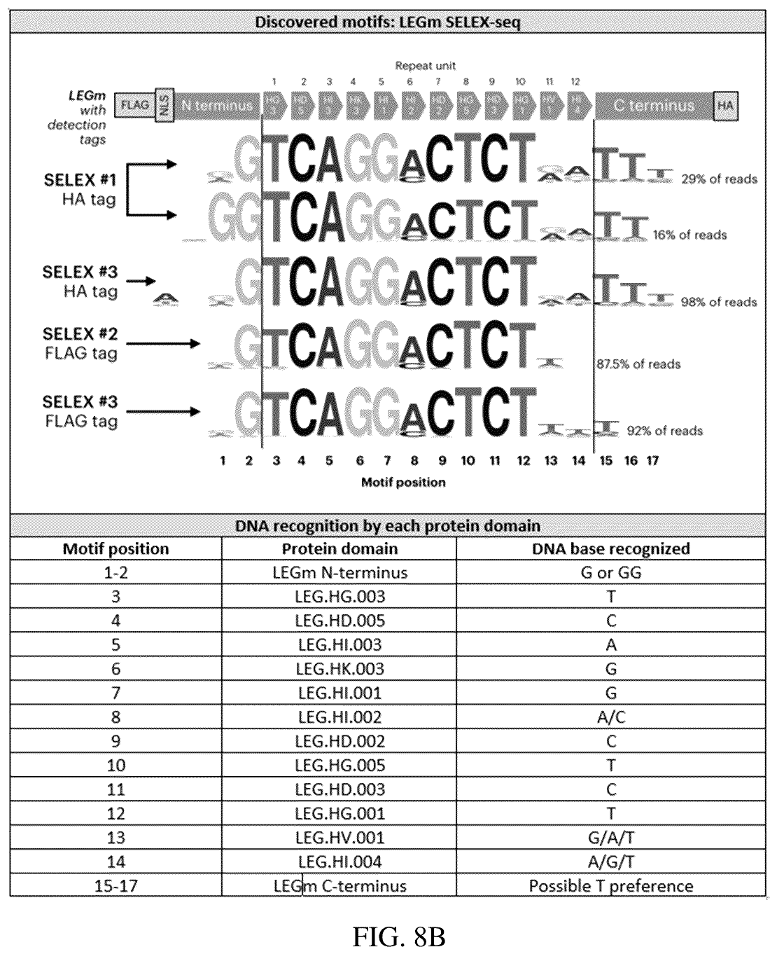

The binding motifs for the LEGq and LEGm proteins as discovered by SELEX-seq are shown in FIGS. 8A and 8B, respectively and the DNA base recognized are listed below:

| Discovered motif: LEGq | | | Discovered motif: LEGm | | |
|---|---|---|---|---|---|
| SELEX DNA recognition | | | SELEX DNA recognition | | |
| by each protein domain | | | by each protein domain | | |
| Motif | Protein | DNA base | Motif | Protein | DNA base |
| position | domain | recognized | position | domain | recognized |
| | LEGq | none | 1-2 | LEGm | G or GG |
| | N-terminus | | | N-terminus | |
| | (SEQ ID | | | (SEQ ID | |
| | NO: 13) | | | NO: 144) | |
| 1 | LEG.RN | G/A | 3 | LEG.HG.003 | T |
| | (SEQ ID | | | (SEQ ID | |
| | NO: 89) | | | NO: 28) | |
| 2 | LEG.HA.001 | A | 4 | LEG.HD.005 | C |
| | (SEQ ID | | | (SEQ ID | |
| | NO: 2) | | | NO: 26) | |
| 3 | LEG.HN.001 | G | 5 | LEG.HI.003 | A |
| | (SEQ ID | | | (SEQ ID | |
| | NO: 3) | | | NO: 32) | |
| 4 | LEG.HG.002 | T | 6 | LEG.HK.003 | G |
| | (SEQ ID | | | (SEQ ID | |
| | NO: 4) | | | NO: 34) | |
| 5 | LEG.NN.001 | G | 7 | LEG.HI.001 | G |
| | (SEQ ID | | | (SEQ ID | |
| | NO: 5) | | | NO: 30) | |
| 6 | LEG.NG.001 | T | 8 | LEG.HI.002 | A/C |
| | (SEQ ID | | | (SEQ ID | |
| | NO: 6) | | | NO: 31) | |
| 7 | LEG.HD.006 | C | 9 | LEG.HD.002 | C |
| | (SEQ ID | | | (SEQ ID | |
| | NO: 7) | | | NO: 23) | |
| 8 | LEG.HG.004 | T | 10 | LEG.HG.005 | T |
| | (SEQ ID | | | (SEQ ID | |
| | NO: 8) | | | NO: 29) | |
| 9 | LEG.HD.001 | C | 11 | LEG.HD.003 | C |
| | (SEQ ID | | | (SEQ ID | |
| | NO: 9) | | | NO: 24) | |
| 10 | LEG.HK.001 | G | 12 | LEG.HG.001 | T |
| | (SEQ ID | | | (SEQ ID | |
| | NO: 33) | | | NO: 27) | |
| | LEGq | none | 13 | LEG.HV.001 | G/A/T |
| | C-terminus | | | (SEQ ID | |
| | (SEQ ID | | | NO: 35) | |
| | NO: 159) | | 14 | LEG.HI.004 | A/G/T |
| | | | | (SEQ ID | |
| | | | | NO: 133) | |
| | | | 15-17 | LEGm | Potentially |
| | | | | C-terminus | T or TT |
| | | | | (SEQ ID | |
| | | | | NO: 159) | |

### Example 17

### In Vivo Base Specificity of Repeats from L. Maceachernii

DNA binding preferences of the LEGm protein (SEQ ID NO:143) to DNA in the chromatin context was assessed by expressing it in vivo and detecting its binding locations in the human genome using the FLAG-seq method.

### FLAG-Seq

In vivo binding of LEGm was detected using the CUT&RUN-seq method (Skene and Henikoff 2017) for detection of FLAG-tagged proteins, an approach referred to as “FLAG-seq.” Briefly, the LEGm construct described above was linearized by restriction digestion and subjected to in vitro transcription using the T7 mScript™ Standard mRNA Production System (CellScript) according to the manufacturer's protocol. One microgram of this LEGm-encoding mRNA was transfected into 500,000 CD3+ T cells using the BTX ECM-830 nucleofection device (Harvard Apparatus) according to the manufacturer's instructions for the specific cell type. Cells were incubated at 37° C. for six hours and subjected to the CUT&RUN-seq protocol using the FLAG tag for detection of the LEGm protein. Isolated DNA fragments from control and LEGm-transfected cells were end-repaired and ligated to sequencing adapters using the SMARTer ThruPLEX DNA-seq kit (Takara) according to the manufacturer's protocol and sequenced on an Illumina NextSeq instrument using standard protocols.

### FLAG-Seq Data Analysis

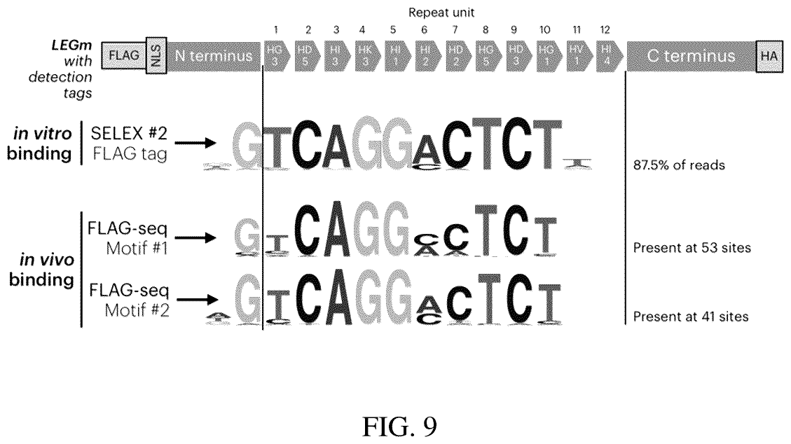

Sequence reads from the NextSeq were de-multiplexed to assign sequences to individual FLAG-seq samples and aligned to the reference human genome using the BWA algorithm (Li and Durbin 2009). Locations of LEGm binding were discovered by using the MACS peak-calling algorithm (Zhang et al. 2008) to compare FLAG-seq signals from control and LEGm-transfected cells, and then mined for enriched sequence motifs using the MEME suite of tools (Bailey et al. 2015). This analysis recovered the same binding motif for LEGm that was observed in multiple SELEX-seq experiments (FIG. 9).

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

For reasons of completeness, certain aspects of the polypeptides, composition, and methods of the present disclosure are set out in the following numbered clauses:

1. A composition comprising a non-naturally occurring modular nucleic acid binding domain derived from an animal pathogen protein (MAP-NBD), wherein the MAP-NBD comprises a plurality of repeat units and wherein a repeat unit of the plurality of repeat units recognizes a target nucleic acid.

2. The composition of clause 1, wherein the animal pathogen protein is derived from a bacterium.

3. The composition of clause 2, wherein the bacterium is selected from the genus of Legionella.

4. The composition of any one of clauses 2-3, wherein the bacterium is L. quateirensis.

5. The composition of any one of clauses 1-4, wherein the repeat unit comprises a consensus sequence of 1xxx211x1xxx33x2x1xxxxxxxxx1 (SEQ ID NO: 19).

6. The composition of any one of clauses 1-5, wherein the target nucleic acid is a single nucleotide, a single base pair, or both.

7. The composition of any one of clauses 1-6, wherein the target nucleic acid is DNA or RNA.

8. The composition of any one of clauses 1-7, wherein the MAP-NBD binds a target nucleic acid sequence.

9. The composition of clause 8, wherein the target nucleic acid sequence is DNA or RNA.

10. The composition of any one of clauses 1-9, further comprising a functional domain.

11. The composition of clause 10, further comprising a naturally occurring or non-naturally occurring linker between the MAP-NBD and the functional domain.

12. The composition of any one of clauses 10-11, wherein the functional domain comprises an enzyme, an activation domain, a repression domain, a biotinylation reagent, a DNA nucleotide modifier, or a fluorophore.

13. The composition of clause 12, wherein the enzyme is a nuclease, a DNA modifying protein, or a chromatin modifying protein.

14. The composition of clause 13, wherein the nuclease is a cleavage domain or a half-cleavage domain.

15. The composition of clause 14, wherein the cleavage domain or half-cleavage domain comprises a type IIS restriction enzyme.

16. The composition of clause 15, wherein the type IIS restriction enzyme comprises FokI or Bfil.

17. The composition of clause 16, wherein FokI has at least 70%, at least 75%, at least 80%, at least 85%, at least 90%, at least 92%, at least 95%, at least 97%, or at least 99% sequence identity to SEQ ID NO: 11.

18. The composition of any one of clauses 16-17, wherein FokI has a sequence of SEQ ID NO: 11.

19. The composition of clause 13, wherein the chromatin modifying protein is lysine-specific histone demethylase 1 (LSD1).

20 The composition of clause 12, wherein the activation domain comprises VP16, VP64, p65, p300 catalytic domain, TET1 catalytic domain, TDG, Ldb1 self-associated domain, SAM activator (VP64, p65, HSF1), VPR (VP64, p65, Rta).

21. The composition of clause 12, wherein the repressor domain comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

22. The composition of clause 12, wherein the DNA nucleotide modifier is adenosine deaminase.

23. The composition of any one of clauses 10-22, wherein the functional domain enables genome editing, gene regulation, or imaging at the genomic locus specified by the modular nucleic acid binding domain.

24. The composition of any one of clauses 1-23, wherein the repeat unit has at least 70%, at least 75%, at least 80%, at least 85%, at least 90%, at least 92%, at least 95%, at least 97%, or at least 99% sequence identity to any one of SEQ ID NO: 2-SEQ ID NO: 10.

25. The composition of any one of clauses 1-24, wherein the repeat unit is any one of SEQ ID NO: 2-SEQ ID NO: 10.

26. The composition of any one of clauses 1-25, wherein the repeat unit is derived from a wild-type protein.

27. The composition of any one of clauses 1-25, wherein the repeat unit comprises a modification of a wild-type protein.

28. The composition of clause 27, wherein the modification enhances specific recognition of a target nucleotide, base pair, or both.

29. The composition of any one of clauses 27-28, wherein the modification comprises 1 to 29 modifications.

30. The composition of any one of clauses 1-29, wherein the animal pathogen protein has at least 70%, at least 75%, at least 80%, at least 85%, at least 90%, at least 92%, at least 95%, at least 97%, or at least 99% sequence identity to SEQ ID NO: 1.

31. The composition of any one of clauses 1-30, wherein the animal pathogen protein is SEQ ID NO: 1.

32. The composition of clauses 1-31, wherein the target nucleic acid sequence is within a PDCD1 gene, a CTLA4 gene, a LAG3 gene, a TET2 gene, a BTLA gene, a HAVCR2 gene, a CCR5 gene, a CXCR4 gene, a TRA gene, a TRB gene, a B2M gene, an albumin gene, a HBB gene, a HBA1 gene, a TTR gene, a NR3C1 gene, a CD52 gene, an erythroid specific enhancer of the BCL11A gene, a CBLB gene, a TGFBR1 gene, a SERPINA1 gene, a HBV genomic DNA in infected cells, a CEP290 gene, a DMD gene, a CFTR gene, an IL2RG gene, or a combination thereof.

33. The composition of any one of clauses 1-32, wherein a chimeric antigen receptor (CAR), alpha-L iduronidase (IDUA), iduronate-2-sulfatase (IDS), or Factor 9 (F9), is inserted upon cleavage of a region of the target nucleic acid sequence.

34. A method of genome editing in a subject, wherein the method comprises:
administering a non-naturally occurring modular nucleic acid binding domain; and
inducing a double stranded break,
wherein the modular nucleic acid binding domain comprises a modular nucleic acid binding domain derived from an animal pathogen protein (MAP-NBD), wherein the MAP-NBD comprises a plurality of repeat units and wherein a repeat unit of the plurality of repeat units recognizes a target nucleic acid.

35. The method of clause 34, further comprising a second MAP-NBD wherein the second MAP-NBD comprises a second plurality of repeat units and wherein a repeat unit of the second plurality of repeat units recognizes a second target nucleic acid.

36. The method of any one of clauses 34-35, wherein the MAP-NBD, the second MAP-NBD, or both further comprise a functional domain.

37 The method of clause 36, wherein the functional domain comprises a cleavage domain or a cleavage half domain.

38. The method of clause 35, wherein the cleavage domain or the cleavage half domain comprises FokI or Bfil.

39 The method of clause 38, wherein the cleavage domain comprises a meganuclease.

40. The method of clause 38, wherein FokI has at least 70%, at least 75%, at least 80%, at least 85%, at least 90%, at least 92%, at least 95%, at least 97%, or at least 99% sequence identity to SEQ ID NO: 11.

41. The method of any one of clauses 38 or 40, wherein FokI has a sequence of SEQ ID NO: 11.

42. The method of clauses 34-41, wherein the target nucleic acid sequence is within a PDCD1 gene, a CTLA4 gene, a LAG3 gene, a TET2 gene, a BTLA gene, a HAVCR2 gene, a CCR5 gene, a CXCR4 gene, a TRA gene, a TRB gene, a B2M gene, an albumin gene, a HBB gene, a HBA1 gene, a TTR gene, a NR3C1 gene, a CD52 gene, an erythroid specific enhancer of the BCL11A gene, a CBLB gene, a TGFBR1 gene, a SERPINA1 gene, a HBV genomic DNA in infected cells, a CEP290 gene, a DMD gene, a CFTR gene, an IL2RG gene, or a combination thereof.

43. The method of any one of clauses 34-42, wherein a chimeric antigen receptor (CAR), alpha-L iduronidase (IDUA), iduronate-2-sulfatase (IDS), or Factor 9 (F9), is inserted upon cleavage of a region of the target nucleic acid sequence.

44. A method of gene regulation in a subject, wherein the method comprises:
administering a non-naturally occurring modular nucleic acid binding domain; and
regulating expression of a gene,
wherein the modular nucleic acid binding domain comprises a modular DNA binding domain derived from an animal pathogen protein (MAP-NBD) and wherein the MAP-NBD comprises a plurality of repeat units and wherein a repeat unit of the plurality of repeat units recognizes a target nucleic acid.

45. The method of clause 44, wherein the MAP-NBD further comprises a functional domain.

46. The method of clause 45, wherein the functional domain comprises an activation domain or a repression domain.

47. The method of clause 46, wherein the activation domain comprises VP16, VP64, p65, p300 catalytic domain, TET1 catalytic domain, TDG, Ldb1 self-associated domain, SAM activator (VP64, p65, HSF1), VPR (VP64, p65, Rta).

48. The method of clause 46, wherein the repressor domain comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

49. The method of clauses 44-48, wherein the target nucleic acid sequence is within a PDCD1 gene, a CTLA4 gene, a LAG3 gene, a TET2 gene, a BTLA gene, a HAVCR2 gene, a CCR5 gene, a CXCR4 gene, a TRA gene, a TRB gene, a B2M gene, an albumin gene, a HBB gene, a HBA1 gene, a TTR gene, a NR3C1 gene, a CD52 gene, an erythroid specific enhancer of the BCL11A gene, a CBLB gene, a TGFBR1 gene, a SERPINA1 gene, a HBV genomic DNA in infected cells, a CEP290 gene, a DMD gene, a CFTR gene, an IL2RG gene, or a combination thereof.

50. The method of any one of clauses 44-49, wherein a chimeric antigen receptor (CAR), alpha-L iduronidase (IDUA), iduronate-2-sulfatase (IDS), or Factor 9 (F9), is inserted upon cleavage of a region of the target nucleic acid sequence.

51. A method of imaging a genomic locus, wherein the method comprises:
administering a non-naturally occurring modular nucleic acid binding domain; and
imaging the subject,
wherein the modular nucleic acid binding domain comprises a modular DNA binding domain derived from an animal pathogen protein (MAP-NBD) and wherein the MAP-NBD comprises a plurality of repeat units and wherein a repeat unit of the plurality of repeat units recognizes a target nucleic acid.

52. The method of clause 51, wherein the MAP-NBD further comprises a functional domain.

53. The method of clause 52, wherein the functional domain is an imaging agent.

54. The method of clause 53, wherein the imaging agent is a fluorescent moiety.

55. The method of clause 54, wherein the fluorescent moiety is GFP or mCHERRY.

56. The method of any one of clauses 38-55, wherein the target nucleic acid is a single nucleotide, a single base pair, or both.

57. The method of any one of clauses 38-56, wherein the target nucleic acid is DNA or RNA.

58. The method of any one of clauses 38-57, wherein the MAP-NBD recognizes a target nucleic acid sequence.

59 The method of clause 58, wherein the MAP-NBD binds the target nucleic acid sequence.

60. The method of any one of clauses 58-59, wherein the target nucleic acid sequence is DNA or RNA.

61. The method of any one of clauses 34-43, 44-50, or 51-60, further comprising a linker between the MAP-NBD and the functional domain.

62. The method of any one of clauses 32-61, wherein the animal pathogen protein is derived from a bacterium.

63. The method of clause 62, wherein the bacterium is selected from the genus of Legionella.

64 The method of any one of clauses 62-63, wherein the bacterium is L. quateirensis.

65. The method of any one of clauses 32-64, wherein the repeat unit comprises a consensus sequence of 1xxx211x1xxx33x2x1xxxxxxxxx1 (SEQ ID NO: 19).

66. The method of any one of clauses 32-65, wherein the repeat unit has at least 70%, at least 75%, at least 80%, at least 85%, at least 90%, at least 92%, at least 95%, at least 97%, or at least 99% sequence identity to any one of SEQ ID NO: 2-SEQ ID NO: 10.

67. The method of any one of clauses 32-66, wherein the repeat unit is any one of SEQ ID NO: 2-SEQ ID NO: 10.

68. The method of any one of clauses 32-67, wherein the repeat unit is derived from a wild-type protein.

69. The method of any one of clauses 32-68, wherein the repeat unit comprises a modification of a wild-type protein.

70. The method of clause 69, wherein the modification enhances specific recognition of a target nucleotide.

71. The method of any one of clauses 69-70, wherein the modification comprises 1 to 29 modifications.

72. The method of any one of clauses 32-71, wherein the animal pathogen protein has at least 70%, at least 75%, at least 80%, at least 85%, at least 90%, at least 92%, at least 95%, at least 97%, or at least 99% sequence identity to SEQ ID NO: 1.

73. The method of any one of clauses 32-66, wherein the animal pathogen protein is SEQ ID NO: 1.

74 The method of any one of clauses 32-73, wherein the genomic locus is in a cell.

75. The method of clause 68, wherein the cell is in a plurality of cells ex vivo, in a human, or in a non-human animal.

76. The method of any one of clauses 51-75, wherein the target nucleic acid sequence is within a PDCD1 gene, a CTLA4 gene, a LAG3 gene, a TET2 gene, a BTLA gene, a HAVCR2 gene, a CCR5 gene, a CXCR4 gene, a TRA gene, a TRB gene, a B2M gene, an albumin gene, a HBB gene, a HBA1 gene, a TTR gene, a NR3C1 gene, a CD52 gene, an erythroid specific enhancer of the BCL11A gene, a CBLB gene, a TGFBR1 gene, a SERPINA1 gene, a HBV genomic DNA in infected cells, a CEP290 gene, a DMD gene, a CFTR gene, an IL2RG gene, or a combination thereof.

77. The method of any one of clauses 51-76, wherein a chimeric antigen receptor (CAR), alpha-L iduronidase (IDUA), iduronate-2-sulfatase (IDS), or Factor 9 (F9), is inserted upon cleavage of a region of the target nucleic acid sequence.

1. (canceled)

2. A recombinant polypeptide comprising a nucleic acid binding domain (NBD) and a heterologous functional domain, the NBD comprising at least three repeat units (RUs) ordered from N-terminus to C-terminus of the NBD to specifically bind to a target nucleic acid, wherein each of the RUs comprises the consensus sequence:
(SEQ ID NO: 154)
(F/L/Y)(D/G/N/S)(A/H/R/S/T/V)(D/E/K/Q)(E/H/Q)
(I/L/V)(I/L/V)(C/H/K/R/S)(I/M/V)(A/V)(A/G/S)
(H/N/R)(A/D/G/I/K/N/S/V)(G)(G)(A/G/S)(H/K/L/
N/R)(N)(I/L)(A/D/E/I/K/V)(A/L/V)(I/M/V)(K/L/
Q/T)(A/D/E/K/L/Q/S)(A/C/F/N/V/Y)(F/H/L/Q/Y)
(A/D/H/P/Q)(A/D/I/K/R/T/V)(F/L)(K/M/Q/R/S)
(D/E/N/S)(F/L/M)(D/E/G/H/K/N)

3. (canceled)

4. The recombinant polypeptide of claim 2, wherein each RU independently comprises a 33-36 amino acid long sequence that is at least 70% identical to:
(SEQ ID NO: 2)
FSSQQIIRMVSHAGGANNLKAVTANHDDLONMG;
(SEQ ID NO: 3)
FNVEQIVRMVSHNGGSKNLKAVTDNHDDLKNMG;
(SEQ ID NO: 4)
FNAEQIVRMVSHGGGSKNLKAVTDNHDDLKNMG;
(SEQ ID NO: 5)
FNAEQIVSMVSNNGGSKNLKAVTDNHDDLKNMG;
(SEQ ID NO: 6)
FNAEQIVSMVSNGGGSLNLKAVKKYHDALKDRG;
(SEQ ID NO: 7)
FNTEQIVRMVSHDGGSLNLKAVKKYHDALRERK;
(SEQ ID NO: 8)
FNVEQIVSIVSHGGGSLNLKAVKKYHDVLKDRE;
(SEQ ID NO: 9)
FNAEQIVRMVSHDGGSLNLKAVTDNHDDLKNMG;
(SEQ ID NO: 33)
FNAEQIVRMVSHKGGSKNLALVKEYFPVFSSFH;
or
(SEQ ID NO: 89)
LGHKELIKIAARNGGGNNLIAVLSCYAKLKEMG'
and comprises base-contacting residues (BCR) selected from HK, HD, HA, HN, HG, NN, NG, RN, HI, HV, RT, HD, SN, HS, GS, or LN, at amino acid positions 12 and 13, respectively, numbered relative to one of SEQ ID NOs: 2-9, 33, and 89.

5. (canceled)

6. (canceled)

7. (canceled)

8. (canceled)

9. (canceled)

10. (canceled)

11. (canceled)

12. (canceled)

13. The recombinant polypeptide of claim 2, wherein each RU independently comprises a 33-36 amino acid long sequence that is at least 70% identical to:
(SEQ ID NO: 131)
FKADDAVRIACRTGGSHNLKAVHKNYERLRARG;
(SEQ ID NO: 132)
FNADQVIKIVGHDGGSNNIDVVQQFFPELKAFG;
(SEQ ID NO: 138)
FSADQVVKIAGHSGGSNNIAVMLAVFPRLRDFG;
or
(SEQ ID NO: 25)
LDRQQILRIASHDGGSKNIAAVQKFLPKLMNFG,
and comprises base-contacting residues (BCR) selected from HK, HD, HA, HN, HG, NN, NG, RN, HI, HV, RT, HD, SN, HS, GS, or LN at amino acid positions 12 and 13, respectively, numbered relative to one of SEQ ID NOs: 25, 131, 132, and 138.

14. (canceled)

15. (canceled)

16. (canceled)

17. (canceled)

18. (canceled)

19. (canceled)

20. The recombinant polypeptide of claim 2, wherein each RU independently comprises a 33-36 amino acid long sequence that is at least 70% identical to:
(SEQ ID NO: 23)
FSAEQIVRIAAHDGGSRNIEAVQQAQHVLKELG;
(SEQ ID NO: 24)
FSAEQIVSIVAHDGGSRNIEAVQQAQHILKELG;
(SEQ ID NO: 26)
FSAEQIVRIAAHDGGSLNIDAVQQAQQALKELG;
(SEQ ID NO: 27)
FSTEQIVCIAGHGGGSLNIKAVLLAQQALKDLG;
(SEQ ID NO: 28)
YSSEQIVRVAAHGGGSLNIKAVLQAHQALKELD;
(SEQ ID NO: 29)
FSAEQIVHIAAHGGGSLNIKAILQAHQTLKELN;
(SEQ ID NO: 30)
FSAEQIVRIAAHIGGSRNIEAIQQAHHALKELG;
(SEQ ID NO: 31)
FSAEQIVRIAAHIGGSHNLKAVLQAQQALKELD;
(SEQ ID NO: 32)
FSAKHIVRIAAHIGGSLNIKAVQQAQQALKELG;
(SEQ ID NO: 34)
FSADQIVRIAAHKGGSHNIVAVQQAQQALKELD;
(SEQ ID NO: 35)
FSAEQIVSIAAHVGGSHNIEAVQKAHQALKELD;
or
(SEQ ID NO: 133)
FSAEQIVRIAAHIGGSRNIEATIKHYAMLTQPP,
and comprises base-contacting residues (BCR) selected from HK, HD, HA, HN, HG, NN, NG, RN, HI, HV, RT, HD, SN, HS, GS, or LN at amino acid positions 12 and 13, respectively, numbered relative to one of SEQ ID NOs: 23-24, 26-32, 34-35, and 133.

21. The recombinant polypeptide of claim 20, wherein the polypeptide comprises an N-terminal domain, wherein the C-terminus of the N-terminal domain is fused to the N-terminus of the first RU of the NBD and/or wherein the polypeptide comprises a C-terminal domain, wherein the N-terminus of the C-terminal domain is fused to the C-terminus of the last RU of the NBD.

22. The recombinant polypeptide of claim 21, comprising a linker amino acid sequence between the C-terminus of the N-terminal domain and the N-terminus of the first RU of the NBD and/or between the N-terminus of the C-terminal domain and the C-terminus of the last RU of the NBD.

23. The recombinant polypeptide of claim 21, wherein the N-terminal domain comprises an amino acid sequence at least 85% identical to the amino acid sequence set forth in SEQ ID NO: 144 or a fragment thereof.

24. (canceled)

25. (canceled)

26. The recombinant polypeptide of claim 21, wherein the C-terminal domain comprises an amino acid sequence at least 85% identical to the amino acid sequence set forth in SEQ ID NO:145 or a fragment thereof.

27. (canceled)

28. (canceled)

29. A recombinant polypeptide comprising a nucleic acid binding domain (NBD) and a heterologous functional domain, the NBD comprising at least three repeat units (RUs) ordered from N-terminus to C-terminus of the NBD to specifically bind to a target nucleic acid, wherein each of the RUs comprises the consensus sequence:
(SEQ ID NO: 156)
YK(P/S)EDIIRLASH(D/G)GGSVNLEA
VLRL(H/N)(P/S)QL(I/T)(G/R)LG

30. (canceled)

31. (canceled)

32. (canceled)

33. (canceled)

34. (canceled)

35. (canceled)

36. (canceled)

37. (canceled)

38. (canceled)

39. The recombinant polypeptide of claim 20, wherein the heterologous functional domain is a polypeptide positioned N-terminal or C-terminal to the NBD.

40. (canceled)

41. (canceled)

42. The recombinant polypeptide of claim 39, wherein the functional domain comprises an enzyme, a transcriptional activator, a transcriptional repressor, or a DNA nucleotide modifier.

43. The recombinant polypeptide of claim 42, wherein the enzyme is a nuclease, a DNA modifying protein, or a chromatin modifying protein, wherein the transcriptional activator comprises VP16, VP64, p65, p300 catalytic domain, TET1 catalytic domain, TDG, Ldb1 self-associated domain, SAM activator (VP64, p65, HSF1), or VPR (VP64, p65, Rta), wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2, or wherein the DNA nucleotide modifier is adenosine deaminase.

44. (canceled)

45. (canceled)

46. (canceled)

47. (canceled)

48. (canceled)

49. (canceled)

50. (canceled)

51. (canceled)

52. (canceled)

53. The recombinant polypeptide of claim 2, wherein the target nucleic acid is within a PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEAI gene, a TTR gene, a NR3CI gene, a CD52 gene, an erythroid specific enhancer of the ECLIIA gene, a CELE gene, a TGFERI gene, a SERPINAI gene, a HEV genomic DNA in infected cells, a CEP290 gene, a DMD gene, a CFTR gene, or an IL2RG gene.

54. The recombinant polypeptide of claim 2, wherein the heterologous functional domain comprises a fluorophore or a detectable tag.

55. A nucleic acid encoding the polypeptide of claim 2.

56. (canceled)

57. (canceled)

58. (canceled)

59. (canceled)

60. (canceled)

61. (canceled)

62. (canceled)

63. (canceled)

64. (canceled)

65. A pharmaceutical composition comprising the polypeptide of claim 2 or a nucleic acid encoding the polypeptide of claim 2; and a pharmaceutically acceptable excipient.

66. (canceled)

67. A method of modulating expression of an endogenous gene in a cell, the method comprising:
introducing into the cell the polypeptide of claim 39 or a nucleic acid encoding the polypeptide of claim 39,
wherein the NBD of the polypeptide binds to a target nucleic acid sequence present in the endogenous gene and the heterologous functional domain modulates expression of the endogenous gene.

68. The method of claim 67, wherein the polypeptide is introduced as a nucleic acid encoding the polypeptide and wherein the nucleic acid is a deoxyribonucleic acid (DNA) or a ribonucleic acid (RNA), wherein optionally the sequence of the nucleic acid is codon optimized for expression in a human cell.

69. (canceled)

70. (canceled)

71. (canceled)

72. The method of claim 67, wherein the functional domain is a transcriptional activator and the target nucleic acid sequence is present in an expression control region of the gene, wherein the polypeptide increases expression of the gene, and optionally, wherein the transcriptional activator comprises VP16, VP64, p65, p300 catalytic domain, TET1 catalytic domain, TDG, Ldb1 self-associated domain, SAM activator (VP64, p65, HSF1), or VPR (VP64, p65, Rta) or wherein the functional domain is a transcriptional repressor and the target nucleic acid sequence is present in an expression control region of the gene, wherein the polypeptide decreases expression of the gene, and optionally, wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

73. (canceled)

74. (canceled)

75. (canceled)

76. The method of claim 67, wherein the gene is a PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEAI gene, a TTR gene, a NR3CI gene, a CD52 gene, an erythroid specific enhancer of the ECLIIA gene, a CELE gene, a TGFERI gene, a SERPINAI gene, a HEV genomic DNA in infected cells, a CEP290 gene, a DMD gene, a CFTR gene, or an IL2RG gene.

77.-117. (canceled)
